(19) United States
(12) Patent Application Publication
(10) Pub. No.: US 2019/0270960 A1
(43) Pub. Date: Sep. 5, 2019

(54) MICROFLUIDIC FILTER DEVICES AND METHODS OF FABRICATING MICROFLUIDIC FILTER DEVICES

(71) Applicant: HysOcean, Inc., San Diego, CA (US)

(72) Inventors: Fanqing Chen, Moraga, CA (US); Robert P. Chebi, San Carlos, CA (US); Binbin Fang, Hayward, CA (US)

(21) Appl. No.: 16/332,184

(22) PCT Filed: Sep. 11, 2017
(86) PCT No.: PCT/US17/50994

§ 371 Date: Mar. 11, 2019

Related U.S. Application Data
(60) Provisional application No. 62/394,096, filed on Sep. 13, 2016.

(51) Int. Cl. C12M 1/00; B01D 21/34; B01D 27/02; B01D 39/14; C12N 13/00; B01D 46/00; C12N 5/00

(52) U.S. Cl. CPC C12M 47/04; B01D 21/34; B01D 27/02; C12N 5/0087; C12N 13/00; B01D 46/0001; C12M 45/07; B01D 39/14

(57) ABSTRACT

Microfluidic filter devices and methods of fabricating such devices. A microfluidic filter device for capturing an object (e.g., a red blood cell) can include a filter structure having a plurality of through holes extending from a first side of the filter structure to a second side of the filter structure and arranged in a repeating pattern, the through holes sized to capture the object. The device further includes a substrate including a plurality of vanes that supports at least a portion of the filter structure, a plurality of electrodes comprising a set of electrodes associated with each through hole, each set of electrodes including at least a pair of electrodes associated with each through hole and aligned with its associated through hole to apply electrical forces to an object captured in the through hole, and electrical connections to each of the plurality of electrodes.

### CROSS REFERENCE TO RELATED APPLICATION

This application claims the benefit of U.S. Provisional Application No. 62/394,096, filed Sep. 13, 2016, which is hereby incorporated by reference in its entirety.

### BACKGROUND

### Technical field

The embodiments disclosed herein relate to methods and devices for isolating, analyzing, manipulating, and extracting objects of interest such as cells or microbeads using a microfluidic filter structure in a microfluidic chip.

### Description of the Related Art

Isolation of cells of interest from cell samples containing both cells of interest and cells not of interest for non-invasive diagnosis presents various challenges. For example isolating circulating fetal cells (CFC) from maternal blood containing other maternal and fetal cells not of interest for non-invasive prenatal diagnosis presents challenges due to the rarity of fetal red cells in the maternal blood. The same problem exists in isolation of rare circulating tumor cells (CTC) from blood for liquid biopsies. In these situations, various approaches have been attempted to extract and analyze cells of interest for downstream genetic analysis and diagnostic assays, but the success and purity of extraction has been very poor. Additionally, throughput of such detection and extraction systems remains low, presenting another challenge in the field of non-invasive testing. For example, some methods of isolating cells of interest utilize cell samples plated or spread on a slide or plate for analyzing, isolating, and extracting cells for further analysis. However, the spreading methods employed present challenges because cells often clump together in more than one layer and overlap with each other, making it very difficult to identify boundaries of each cell to determine if the cell is a cell of interest. Other methods often capture cells of interest and cells that are not of interest, and these methods fail to precisely and accurately identify cells not of interest. Further, these methods do not provide for the precise and controllable removal of not of interest independent surrounding cells.

### SUMMARY

Some of the present embodiments may include a multi-layer microfluidic device configured to capture and isolate cells of interest using morphology-based isolation. In some aspects, the multi-layer microfluidic device may include a first layer comprising a microfluidic filter structure, such as a microfluidic filter material or microfluidic filter membrane, disposed on a second layer comprising a support structure, such as a substrate. For example, the filter membrane may be deposited as a thin film onto the substrate or the filter membrane may be spun onto the substrate. A microfluidic chip can include one or more of the multi-layer microfluidic devices. Although the above-described embodiment is a dual-layer microfluidic device, other embodiments are possible. For example, multi-layer microfluidic devices described herein can include a microfluidic filter structure that includes 1, 2, 3, or more layers. For another example, multi-layer microfluidic devices described herein can include a support structure having one or more layers.

Embodiments described herein can include at least one microfluidic filter structure configured to isolate cells of interest from a sample containing cells of interest while simultaneously positioning the cell in a distinct, precisely-defined location of the filter structure that is spatially separate from other distinct, precisely-defined locations of the filter structure. Embodiments of microfluidic devices described herein include a filter structure, such as a microfluidic filter material or microfluidic filter membrane, that automatically creates a monolayer of cells of interest as a stained sample flows over or through the microfluidic device. In some aspects, the filter structure includes a filter membrane comprising multiple through holes specifically shaped and dimensioned to capture cells of interest while permitting cells not of interest to pass through the through holes in the filter membrane and thereby remain uncaptured. The through holes are specifically arranged in a predetermined and repeating grid-like pattern.

Through holes described herein include a first opening on a first side of a filter membrane, a second opening on a second, opposing side of the filter membrane, and a passageway through the filter membrane between the first and second openings. The passageway can include one or more sidewalls within the interior of the filter membrane. Through holes described herein allow objects to translocate through a filter membrane. For example, through holes can allow an object initially present on one side of the filter membrane to translocate through the membrane to a region on the opposing side of the membrane. In some cases, through holes do not allow an object of interest to pass through the membrane, and retain the object of interest on one side of the membrane. Objects retained in this manner can create a monolayer of objects of interest on one side of the filter membrane.

The shape of the opening of a through hole formed in filter membranes described herein can vary. As will be described in detail below, the opening of a through hole on a first side of the filter membrane can have a circular shape. Other shapes are possible. For example, in some implementations the filter membrane includes through holes with openings that are generally rectangular in shape. As will be described in detail below, openings having a rectangular shape can advantageously facilitate flow of the sample through the filter membrane and capture of objects of interest in the filter membrane. Additionally, openings of through holes described herein may also include chamfered or rounded corners that advantageously facilitate the smooth flow of a sample containing cells of interest through the through holes. In one non-limiting example, an opening of a through hole in a first side of a filter membrane has a generally rectangular shape with four corners or edges, and one or more of the corners are chamfered or rounded. The opening of the through hole in the second, opposing side of the filter membrane may also have a generally rectangular shape, and may or may not include chamfered or rounded corners.

Embodiments of filter membranes described herein can include through holes with passageways or side walls that are generally perpendicular to the first and second sides of the filter membranes. In other embodiments of filter membranes described herein, through holes have tapered sidewalls that extend through the interior of the filter structure between a first and second side of the filter membrane at an angle. In one non-limiting embodiment, the features of an angled sidewall of a through hole serve dual functions: one being a physical hydrodynamic trap preventing captured cells or beads of further lateral or directional movement, and the other being a filtering or isolating membrane. If the through holes do not include tapered side walls, the through holes may only serve to prevent certain cells from flowing through the filtering membrane, but would not serve as a hydrodynamic trap, or capturing grid, for cells or beads of targeted size, thus retaining and immobilizing them within, or partially within, the through holes. In one non-limiting example of a circular through hole, the thickness of the membrane and the angle of the through hole side walls determine the capturing and immobilizing characteristics of the filter membrane, and the smallest diameter, at the bottom of the through hole, determines its filtering or isolating properties. Similar effects can be achieved in non-circular through holes by independently selecting the angle of the tapered side wall and the smallest dimension (measured along the x-axis and the y-axis) of the through hole.

In one non-limiting aspect, a through hole includes a sidewall that is tapered at an angle relative to a line that is generally perpendicular to the first and second side of the filter membrane. Further, filter membranes described herein may be constructed or formed of a material that is mechanically and chemically stable, chemically and electronically inert, hydrophilic and transparent in at least the visual spectrum of light. In some aspects, the support substrate may further comprise support vanes formed out of or into the substrate material. The support vanes can be configured to provide structural integrity to a filter membrane disposed adjacent to the support substrate, and may define a shape and size of one filter region in the filter membrane. In embodiments where portions of a filter membrane are suspended over, but are not in direct contact with, a support substrate, the support vanes can provide structural integrity to the portions of the filter membrane that are suspended over the support substrate. In some embodiments, vanes in support structures described herein may also define a field-of-view (“FOV”) of an imaging cytometry process, where the shape and size of the vane-defined FOV generally matches the shape and size of one filter region in filter membrane.

Embodiments of microfluidic chips described herein can manipulate an object captured in a particular through hole by applying a voltage bias to a plurality of electrodes associated with the through hole, providing an enhanced and selective filtration device and method. Manipulating the object in the through hole can include changing the physical dimension of the object or part of the object (for example, stretching, deforming, or lengthening the object or part of the object) and/or discarding the object from the filter membrane (for example, fragmenting or destroying the object). The plurality of electrodes associated with each through hole can include a set of electrodes associated with each through hole and aligned with each through hole to apply electrical forces to an object captured in the respective through hole. The set of electrodes includes at least a pair of electrodes precisely aligned with a respective through hole and configured to apply electrical forces to an object captured in the respective through hole. In some cases described in detail below, a first electrode of the pair of electrodes is positioned on a first side of the respective through hole, and a second electrode of the pair of electrodes is positioned on a second, opposing side of the respective through hole. In other cases described in detail below, a first electrode and a second electrode of the pair of electrodes are both positioned on a first side of the respective through hole. Each pair of electrodes is associated with a single through hole having a distinct, precisely-defined location in the filter membrane, such that the pair of electrodes associated with each through hole also has a distinct, precisely-defined location in the filter membrane. This enables precise control of electrical signals applied to each pair of electrodes that are configured to apply an electrical force to a particular through hole, independent of other electrode pairs configured to apply an electrical force to other through holes in the filter membrane.

In some embodiments of filter membranes disclosed herein, where a through hole has captured an object, the electrical signal that is applied to the through hole by an electrode pair is also applied to the object captured in the through hole. The captured object can be an object that is not of interest, such as a cell or cellular material that is not a cell of interest. Embodiments of microfluidic chips described herein can apply a voltage bias to the captured object, and precisely control the magnitude of that voltage bias applied to that captured object, such that the object associated with a specific through hole can be manipulated. For example, in a case where the object captured in the through hole is a cell, the object can be manipulated by applying electrical forces that attract the cell or a portion of the cell in a particular direction, by applying electrical forces that repel the cell or a portion of the cell in a particular direction, by applying electrical forces that fragment the cell or a portion of the cell, or by applying electrical forces that destroy the cell, based on the voltage bias applied to the electrode pair specifically associated with the through hole. Microfluidic chips described herein can control the voltage bias applied to each electrode pair, and thus each through hole, independent of other electrode pairs and/or through holes in the device, thereby enhancing cell sorting and filtration device, wherein selected cells captured in the filter membrane are targeted for removal from the filter membrane while other cells captured in the filter membrane remain unaffected. In one example embodiment, captured cells that are identified as not being of interest are selectively targeted and removed from the filter membrane without affecting, removing, or destroying other captured cells that are identified as being cells of interest.

One innovation includes a device, including a filter structure having a plurality of through holes extending from a first side of the filter structure to a second side of the filter structure and arranged in a repeating pattern, each of the through holes having a first opening on the first side of the filter structure, a second opening on the second side of the filter structure, and a passageway through the filter structure between the first opening and the second opening, the first and second openings sized to capture an object in the through hole. The device further includes a substrate including a plurality of vanes that supports at least a portion of the filter structure, the filter structure disposed relative to the plurality of vanes such that the second side of the filter structure is adjacent to the plurality of vanes, a plurality of electrodes comprising a set of electrodes associated with each through hole, each set of electrodes including at least a pair of electrodes associated with each through hole, each set of electrodes aligned with its associated through hole to apply electrical forces to an object captured in the through hole, each set of electrodes and associated through hole having a distinct, precisely-defined location in the filter structure, and electrical connections to each of the plurality of electrodes, the electrical connections and plurality of electrodes collectively configured to communicate electrical signals to the plurality of electrodes from a controller connected to the device for independently controlling the application of electrical forces through each set of electrodes to an object in the associated through hole. In various implementations, the device can include one or more additional aspects/features. For example, for each pair of electrodes and associated through hole, a first electrode of the pair of electrodes can be positioned on the through hole on the first side of the filter structure, and a second electrode of the pair of electrodes can be positioned on the through hole on the second side of the filter structure. For each pair of electrodes and associated through hole, both a first electrode and a second electrode of the pair of electrodes may be positioned on the first side of the filter structure. For each pair of electrodes and associated through hole, both a first electrode and a second electrode of the pair of electrodes may be positioned on the second side of the filter structure. Each of the electrodes of the pair of electrodes can be ring-shaped. Each of the through holes can be oval-shaped. Each of the electrodes of the pair of electrodes can be diamond-shaped. Each of the through holes can be circular-shaped. In some implementations, the set of electrodes includes three electrodes. In some implementations, the set of electrodes includes four electrodes. In some implementations, the set of electrodes is configured to apply an electrical force to an object in the associated through hole to fragment an object in the through hole. In some implementations, the set of electrodes is configured to apply an electrical force to an object in the associated through hole to change the shape of an object in the through hole. In some implementations, the set of electrodes is configured to apply an electrical force to an object in the associated through hole to repel an object from the through hole. In some implementations, the set of electrodes is configured to apply an electrical force to attract an object in to the associated through hole. In some implementations the electrical connections include a column connection structure including a column contact pad electrically connected to a column connection line, and a plurality of column lead lines electrically connected to the column connection line, the plurality of column lines each connected to at least one electrode aligned with each through hole. In some implementations,

the electrical connections comprise a row connection structure including a row contact pad electrically connected to a row connection line, a plurality of row lead lines electrically connected to the row connection line, the plurality of row lines connected to at least one electrode aligned with each through hole. In some implementations, the filter structure is formed on the substrate. In some examples, the filter structure has a thickness in the range of about 1 μm to about 20 μm. In some examples, the second opening is smaller than the first opening, and wherein the first and second openings have a first dimension of between about 4 μm and about 10 μm and a second dimension of between about 4 μm and about 10 μm.

Another innovation includes a device, having means for capturing a plurality of red blood cell sized objects each in one of a plurality of holes, the means for capturing having a first side and a second side and arranged in a known pattern, means for supporting the means for capturing, the second side of the means for capturing disposed adjacent to the means for supporting, and means for applying an independently controllable electrical force to each one of an associated one of the plurality of holes. In some implementations, the means for applying an electrical force is positioned on the first side of the means for capturing and on the second side of the means for capturing. In some implementations, the means for applying an electrical force is positioned on the first side of the means for capturing. In some implementations, the mans for applying an electrical force is positioned on the second side of the means for capturing.

In some implementations, the means for applying an independently controllable electrical force to each of the plurality of holes are ring-shaped electrodes, and the plurality of holes can be oval-shaped. In some implementations, the means for applying an independently controllable electrical force are diamond-shaped electrodes, and the plurality of holes may be circular-shaped. In some implementations, applying an independently controllable electrical force includes a plurality of electrodes including a set of electrodes associated with each of the plurality of holes, the set of electrodes including a pair of electrodes. In some devices, the set of electrodes includes three electrodes. In some devices, the set of electrodes includes four electrodes. In some implementations, the set of electrodes is configured to apply an electrical force to an object in the associated hole to fragment an object in the through hole. In some implementations, the set of electrodes is configured to apply an electrical force to an object in the associated hole to change the shape of the object. In some implementations, the set of electrodes is configured to apply an electrical force to an object in the associated through hole to repel the object from the hole. In some implementations, the set of electrodes is configured to apply an electrical force to attract an object to the associated hole.

Another innovation includes a method of capturing an object in a through hole, the method including capturing an object in a through hole of a device including a filter structure having plurality of through holes extending from a first side of the filter structure to a second side of the filter structure and arranged in a repeating pattern, each of the through holes having a first opening on the first side of the filter structure, a second opening on the second side of the filter structure, and a passageway through the filter structure between the first opening and the second opening, the first and second openings sized to capture an object in the through hole, the device further including a substrate having a plurality of vanes that supports at least a portion of the filter structure, the filter structure disposed relative to the plurality of vanes such that the second side of the filter structure is adjacent to the plurality of vanes, applying an electrical force to the captured object using a plurality of electrodes comprising a set of electrodes associated with each through hole, each set of electrodes including at least a pair of electrodes associated with each through hole, each set of electrodes aligned with its associated through hole to apply electrical forces to the object captured in the through hole, each set of electrodes and associated through hole having a distinct, precisely-defined location in the filter structure. In some implementations, the electrical force includes applying an electrical force to an object in the associated through hole to fragment an object in the through hole. In some implementations, the electrical force includes applying electrical force to an object in the associated through hole to change the shape of an object in the through hole. In some implementations, the electrical force includes applying an electrical force to attract an object in to the associated through hole. In some implementations, applying the electrical force comprises applying electrical force to an object in the associated through hole to repel an object from the through hole.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1A illustrates a perspective view of a first side of one embodiment of a microfluidic device for capturing and positioning cells of interest according to the present disclosure.

FIG. 1B illustrates a perspective view of a second, opposing side of the microfluidic device illustrated in FIG. 1A.

FIG. 2 illustrates another embodiment of a microfluidic device for capturing and positioning cells of interest according to the present disclosure.

FIG. 3A illustrates a schematic partial cross-section side view of an embodiment of a microfluidic device having a filter membrane including electrically-controllable through holes according to the present disclosure.

FIG. 3B illustrates a schematic partial cross-section side view of another embodiment of a microfluidic device having a filter membrane including electrically-controllable through holes according to the present disclosure.

FIG. 4 is an example flow diagram illustrating one method of capturing, isolating, analyzing, and harvesting cells of interest using a microfluidic device according to the present disclosure.

FIG. 5 is an example flow diagram illustrating one process for fabricating a microfluidic device having a filter membrane including electrically-controllable through holes according to the present disclosure.

FIGS. 6A, 7A, 8A, 9A, 10A, 11A, 12A, 13A, 14A, 15A, 16A, 17A, 18A, 19A, 20A, 21A, and 22A are schematic illustrations of various stages of fabricating one example microfluidic device according to the example process described with reference to FIG. 5.

FIGS. 6B, 7B, 8B, 9B, 10B, 11B, 12B, 13B, 14B, 15B, 16B, 17B, 18B, 19B, 20B, 21B, and 22B are schematic illustrations of various stages of fabricating another microfluidic device according to the example process described with reference to FIG. 5.

FIG. 23 illustrates a schematic partial top down view of another embodiment of a microfluidic device having a filter membrane including electrically-controllable through holes according to the present disclosure.

FIG. 24 is a flow diagram illustrating an example process for fabricating a microfluidic device having a filter membrane including electrically-controllable through holes as described with reference to FIG. 23.

FIGS. 25A through 25I are schematic illustrations of various stages of fabricating the microfluidic device described with reference to FIG. 23.

FIG. 26 is a flow diagram illustrating another example process for fabricating a microfluidic device having a filter membrane including electrically-controllable through holes according to the present disclosure.

FIGS. 27A through 27K are schematic illustrations of various stages of fabricating the microfluidic device described with reference to FIG. 26.

FIG. 28A and FIG. 28B are schematic illustrations of still another example process of fabricating a microfluidic device according to the present disclosure.

### DETAILED DESCRIPTION OF CERTAIN ILLUSTRATIVE EMBODIMENTS

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

Moreover, the terms top, bottom, over, under and the like in the description and the claims are used for descriptive purposes and not necessarily for describing relative positions. It is to be understood that the terms so used are interchangeable under appropriate circumstances and that the embodiments described herein are capable of operation in other orientations than described or illustrated herein.

As used herein, the singular forms “a,” “an,” and “the” include plural references unless indicated otherwise. For example, “a” filter membrane includes one or more filter membranes. As used herein, the term “microfluidic device” or “microfluidic chip” generally refers to a device through which materials, particularly fluid borne materials, such as liquids, can be transported, in some embodiments on a micro-scale, and in some embodiments on a nanoscale. Thus, microfluidic chips described herein can include microscale features, nanoscale features, and combinations thereof. The samples delivered on such a device may be fluids alone or fluids with suspended components such as cells and particles.

An exemplary microfluidic chip can include structural or functional features dimensioned on the order of a millimeter-scale or less, which are capable of manipulating a fluid at a flow rate on the order of 5 mL/min or less. The size and shape of microfluidic chips described herein can be selected based on the needs of the particular application for which the microfluidic chip is intended. In one example, a microfluidic chip includes a plurality of filters arranged in a grid-like pattern. The size and shape of the filters fabricated in the microfluidic chip can be selected based on the needs of the particular application for which the microfluidic chip is intended. In another example, a microfluidic chip includes a single filter membrane supported by a substrate that includes vanes, where the vanes define regions of the filter membrane. In still another example, a microfluidic chip includes a single filter membrane supported by a substrate that does not include vanes. In some cases, a microfluidic chip includes additional features such as, but not limited to channels, fluid reservoirs, reaction chambers, mixing chambers, separation regions, and supporting structures.

A microfluidic chip can exist alone or can be a part of a microfluidic system which, for example and without limitation, can include: pumps and valves for introducing fluids, e.g., samples, reagents, buffers and the like, into the system and/or through the system; detection equipment or systems; data storage systems; and control systems for controlling fluid transport and/or direction within the device, monitoring and controlling environmental conditions to which fluids in the device are subjected, e.g., temperature, pressure, current, and the like using sensors where applicable. The valves and flow in such systems may be pressure or vacuum driven.

As used herein, the terms “filter” and “filter membrane” refer to a material that separates objects of interest from other objects that are not of interest. Embodiments of filter membranes disclosed herein can obtain cells of interest using morphology-based isolation. Methods and devices disclosed herein advantageously use a filter membrane integrated in a microfluidic device, such as a microfluidic chip. In embodiments described herein, a filter membrane separates objects of interest by retaining objects of interest in through holes in the filter membrane, while objects that are not of interest pass through the through holes which are hydrodynamic traps in the filter membrane. The objects of interest can be, but are not limited to, cells, beads, or microbeads. Embodiments of filter membranes described herein can include a single-layer of material, or include multiple layers, such as two, three, or more layers.

Isolating cells of interest can include capturing a cell in a filter membrane while simultaneously positioning the cell in a distinct, precisely-defined location of the filter membrane that is spatially separated from other distinct, precisely-defined locations of the filter membrane. The sample may contain non-cellular matter and/or cells that are not of interest, in addition to cells of interest. Embodiments of the filter membranes described herein capture some, most, or all of the cells of interest, such that the cells of interest may be isolated from samples containing numerous cells, at least some of which may be cells not of interest. It will be understood that filter membranes described herein are not limited to capture cells and microbeads, however, such that filter membranes can capture other types of objects contained in a sample having objects of interest with physical characteristics (for example, morphology, size, etc.) that differ from physical characteristics of objects that are not of interest. In some embodiments, the filter membrane can also be used in imaging devices and cytometry processes to detect the precise location of cells that have been captured in the filter membrane, assess the characteristics of captured cells to determine if they are cells of interest, and harvest or pluck cells that have been determined to be of interest for downstream analysis, such as genetic and/or diagnostic analysis.

As used herein, the term “through hole” refers to an opening or recess extending through a structure, such as a filter membrane. Filter membranes described herein may comprise multiple through holes specifically shaped and dimensioned to capture and retain cells of interest while permitting cells not of interest to pass through the through holes in the filter membrane and thereby remain uncaptured. For example, methods and devices disclosed herein may be used for fetal cell sorting and isolation from maternal blood samples for non-invasive prenatal diagnosis. In one aspect, methods and devices disclosed herein isolate and analyze such cells for downstream genetic analysis and diagnostic assays.

In one example, the structure includes a first side and a second side, and a through hole includes sidewalls that extend entirely through the structure between the first and the second side. Through holes allow objects to translocate through the structure. For example, through holes can allow an object initially present on one side of the structure to translocate through the structure to a region on the opposing side of the structure. In some cases, through holes do not allow an object to pass through the structure, and retain the object on one side of the structure. Objects that do not translocate through a through hole and are retained in the through hole can be positioned partially or entirely within a through hole. Through holes described herein can be specifically shaped and dimensioned to separate objects of interest from other objects that are not of interest. Through holes may also be referred to as pores, wells, hydrodynamic traps, filter holes, or other terms representing a passageway through a filter membrane, however, these features will be referred to as “through holes” throughout this disclosure. In embodiments described herein, the through holes facilitate the separation and retention of objects from objects not of interest. The through holes can be designed to have specific dimensions corresponding to the shape and size of the objects of interest. In this way, single instances of objects of interest (e.g., a single cell) can be captured in a through hole, while permitting objects not of interest to either be passed entirely through the through hole or inhibited from entering (or being retained within) the through hole. As described above, objects of interest can be, but are not limited to, cells, beads, or microbeads. Through holes may be designed in any shape or size, for example they can have generally circular, rectangular, oval, or other cross-sectional shapes. The shape and size of each through hole may be determined based on the objects of interest being captured by the filter membrane.

Embodiments of integrated microfluidic devices disclosed herein may also comprise a plurality of electrodes associated with each through hole of a plurality of through holes, wherein each plurality of electrodes associated with a specific through hole is precisely aligned with the specific through hole and configured to apply electrical forces to an object captured in the specific through hole. As will be described in greater detail below, the number of electrodes associated with each through hole can vary. In one embodiment, two electrodes are aligned with a single through hole and configured to apply electrical forces to an object captured in the through hole. In other embodiments, three, four, or more electrodes are aligned with a single through hole and configured to apply electrical forces to an object captured in the through hole. As used herein, the terms “electrode/through hole pair” refers to a through hole in a filter membrane and the plurality of electrodes associated with and configured to apply an electrical force to that through hole (and any object that may be captured in that through hole). Each electrode/through hole pair also includes electrical conducting lines configured to communicate electrical signals to the plurality of electrodes from a controller. Each plurality of electrodes is associated with a single through hole having a distinct, precisely-defined location in the filter membrane, such that the plurality of electrodes associated with each through hole also has a distinct, precisely-defined location in the filter membrane. This enables precise control of electrical signals applied to each electrode associated with a single through hole independent of other electrodes associated with other through holes in the filter membrane. In this way, an electrical signal, e.g., a voltage bias, can be applied across each through hole and the electrical signal can be independently controlled for each through hole.

In instances where a through hole has captured a cell, microbead, or other object (whether it be an object of interest or an object not of interest), the electrical signal (e.g., voltage bias) that is applied to the through hole is also applied to the object, such as a cell, captured in the through hole. Embodiments of microfluidic chips described herein can apply a voltage bias to the captured cell, and precisely control the magnitude of that voltage bias applied to that captured cell, such that the cell associated with a specific through hole can be manipulated, e.g., electrical forces attract the cell or a portion of the cell in a particular direction, electrical forces repel the cell or a portion of the cell in a particular direction, electrical forces fragment the cell, or electrical forces destroy the cell based on the voltage bias applied to the one or more electrodes associated with the through hole. The significance of the hydrodynamic aspects of the filter membrane manifested in the tapered sidewall angles comes into effect here, specifically with the fact that applied voltages for the electrode functionality would not have to be unnecessarily applied for long time intervals to captured cells of interest for continued retention. Localized resistive heating emitted by the electrodes is a possible outcome of continued applied voltages to conductive electrodes, and localized heating could be detrimental to good, desired, and sought after rare cells of choice. Hence, the hydrodynamic trapping effect of the filter holes minimizes the need to unnecessarily or excessively activate the electrodes beyond their initial guiding effects (i.e. through exerting attractive or repulsive forces) towards the filter through holes, which then act as an effective capturing grid without any further electrical requirements from the through holes neighboring electrodes. This characteristic of filters systems and methods described herein results in minimizing or eliminating any potential thermal damage effects on captured desired cells.

In one example implementation, an object captured in a through hole is determined to be an object not of interest. The plurality of electrodes associated with the through hole can apply a voltage bias to deform the object (for example, stretch, lengthen, or change the cross sectional diameter of a portion of the object), allowing the entire object to pass through the through hole and out of the filter membrane, thereby clearing the through hole entirely. Alternatively, the plurality of electrodes associated with the through hole can apply a voltage bias and fragment the object in such a way that some or all of the fragments pass through the through hole and out of the filter membrane, thereby clearing the through hole partially or entirely. Microfluidic chips described herein can control the voltage bias applied to each electrode/through hole pair independent of other electrode/through hole pairs, thereby enhancing cell sorting and filtration in a single device, wherein selected cells captured in the filter membrane are targeted for removal from the filter membrane while other cells captured in the filter membrane remain unaffected. It is understood that aspects and embodiments of this disclosure include “consisting” and/or “consisting essentially of” aspects and embodiments.

In the following description, specific details are given to provide a thorough understanding of the examples. However, it will be understood by one of ordinary skill in the art that the examples may be practiced without these specific details. For example, electrical components/devices may be shown in block diagrams in order not to obscure the examples in unnecessary detail. In other instances, such components, other structures, and techniques may be shown in detail to further explain the examples.

Other objects, advantages, and features of the present disclosure will become apparent from the following description taken in conjunction with the accompanying drawings.

Integrated Microfluidic Chip with Filter Membrane

Integrated microfluidic chips for non-invasive isolation of cells (such as, but not limited to, fetal nucleated red blood cells (“RBCs”)) are described herein. The integrated microfluidic chips can include a single filter or a plurality of filters. In embodiments of the microfluidic chip that include a single filter, the filter can include a sheet or layer of filter material (“a filter membrane”) supported by a substrate. Filter membranes described herein can include a single sheet or layer of material, or can include a plurality of sheets or layers of material. In embodiments of microfluidic chips that include a plurality of filters, the plurality of filters can be arranged in a grid-like structure. Some embodiments of the microfluidic chips described herein can also include a binding moiety or affinity molecule. For example, in systems designed to capture fetal nucleated RBCs, the system can include a binding moiety or affinity molecule that specifically binds to a cell-specific antigen or a non-fetal cell-specific antigen for positive selection of fetal cells or negative selection of unwanted cells.

In some embodiments, the integrated microfluidic chip may comprise at least one filter membrane that is transparent and visualizable under a microscope. The filter comprises multiple through holes that are arranged in a repeating grid pattern and are configured to capture and retain and simultaneously position cells of interest in precisely-defined, clearly-distinguishable locations on the filter membrane (each location corresponding to a single through hole). In some embodiments, the through holes are specifically arranged in a regular and repeating grid pattern where each through hole can be precisely located based on a unique, predetermined X, Y coordinate on the filter membrane. In some embodiments, each filter membrane may include several thousand through holes (e.g., 8,000 or more), thus enabling the capture and imaging of several thousand cells.

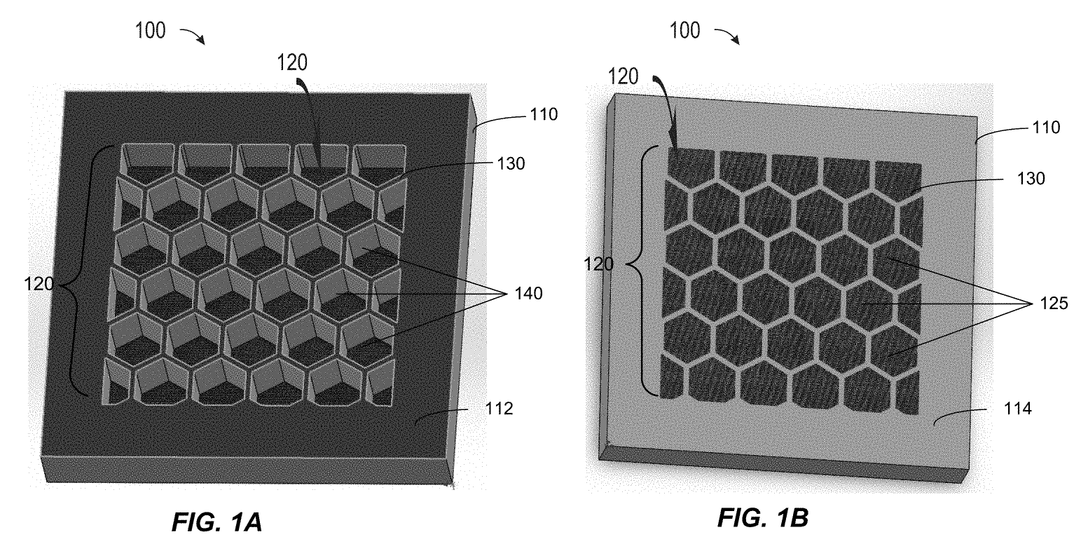

FIGS. 1A and 1B illustrate a first side view and a second side view, respectively, of an exemplary microfluidic chip 100 according to one embodiment. In this non-limiting example, the microfluidic chip 100 is a dual layer structure comprising a support layer and a filter layer. In this case, the support layer includes a substrate 110 and the filter layer includes a filter membrane 120. The substrate 110 includes a first side 112 and an opposing, second side 114. As will be described in detail below, the substrate 110 also includes vanes 130 that extend between the first side 112 and the second side 114. In the illustrated example, the filter membrane 120 is disposed adjacent to, suspended over, and supported by the side 112 of the substrate 110. Portions of the filter membrane 120 are supported by portions of the vanes 130 located on the side 112. In FIG. 1B, for example, the vanes 130 supporting the filter membrane 120 are visible through the filter membrane 120. The vanes 130 define hexagonal-shaped filter regions 125 of the filter membrane 120. Regions 125 having different shapes are possible. In other embodiments (not illustrated in FIG. 1B), a microfluidic chip includes a plurality of hexagonal-shaped filter membranes 120, each filter membrane 120 disposed on or within one hexagonal-shaped region 125 of the substrate 110.

The substrate 110 can be formed of any suitable material and have any suitable dimension to support the filter membrane 120. In some cases, the substrate 110 is a silicon wafer. The silicon wafer can be a commercially available, conventionally-sized wafer that is processed to obtain desired dimensions for the substrate 110. For example, a standard silicon wafer can be thinned down to have a thickness of approximately 400 microns. The thickness of the support material 110 can be selected based on the needs of the particular application for which the microfluidic chip is intended.

The filter membrane 120 includes a plurality of through holes arranged in a regularly-repeating pattern, where each through hole is located a distinct, precisely-defined x, y location of the filter membrane 120. The size, shape, and relative spacing of each through hole can be specifically selected based on the object of interest (for example, a cell of interest) that the filter membrane 120 is designed to capture, such that a single cell of interest is captured in each through hole. The through holes can have openings that are generally rectangular in shape, generally circular in shape, or any other suitable shape.

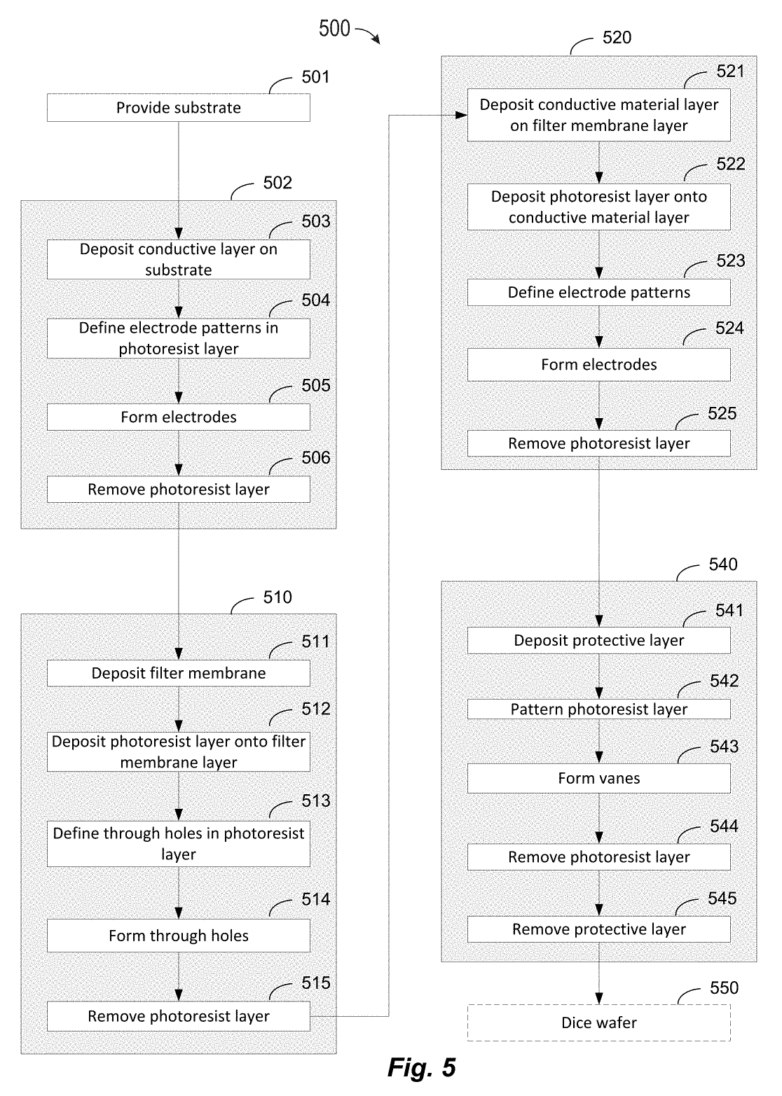

The filter membrane 120 can be formed by any suitable means, as will be described in more detail below with reference to FIGS. 5 through 22B. In one non-limiting aspect, the filter membrane 120 is formed by depositing a very thin layer or layers of material onto the substrate 110. The filter membrane 120 can be formed to have any suitable thickness for the particular application of the microfluidic chip 100. In some cases, the filter membrane 120 is disposed on, disposed adjacent to, or suspended over a top or bottom surface of the substrate 110 and has a thickness of greater than or equal to 5 microns as measured along a z-axis of the filter membrane. For example, the filter membrane 120 can have a thickness of approximately 20 microns. In other examples, the filter membrane 120 has a thickness of about 1 micron, about 2 microns, about 3 microns, about 4 microns, or about 5 microns as measured along a z-axis of the filter membrane. Very thin filter membranes formed according to methods described herein are still relatively strong for their thickness and are advantageously rigid enough to withstand pressures associated with a sample fluid flowing through the filter membrane. These characteristics can be particularly beneficial in applications where more than one sample is applied to a single filter membrane, or in applications where a sample must be applied to a filter membrane at relatively high pressure to ensure efficient and accurate capture of cells of interest in the filter membrane.

The filter membrane 120 may be made from similar materials or different materials as the substrate 110. In example implementations, filter membranes described herein include silicon oxynitride, such as but not limited to SiON or SiO₂. However, any material may be suitable that provides the transparency sought and the requested strength and physical properties for the intended cell capturing application. For example, in some embodiments, the filter material 120 is transparent to light in the visual spectrum (e.g., wavelengths from approximately 400 nanometers to approximately 700 nanometers). In some embodiments, the filter material 120 is transparent to light beyond the visible spectrum, including, but not limited to, light having wavelengths in the near infra-red (NIR) and near ultra-violet (NUV) spectrums. One non-limiting advantage of filter membranes including transparent materials is that cells captured in the filter membrane can be imaged from either side of the substrate 110, for example from the first side 112 or the second side 114 of the substrate 110.

In some embodiments, the filter membrane 120 includes a material or materials that do not fluoresce under illumination by a light source and/or that suppresses background fluorescence. In some embodiments, before, during, or after capture and isolation of cells in the filter membrane, the cells may be labeled or stained with nuclear stains, biomarkers, and/or fluorescent dyes. Such fluorescent molecules or dyes may produce a corresponding light signature or spectrum upon being illuminated or excited by a light source having a particularly corresponding wavelength of light. Thus, using specific fluorescent molecules or dyes may produce an indicator of a particular nucleic acid, antibody or antibody-based fragment probe present on or within a captured cell, which may be imaged using a microscope or other imaging platform. One non-limiting advantage of suppressing or negating the background fluorescence of the filter membrane 120 itself is that total background fluorescence is kept low to avoid interfering with imaging of fluorescent- or light-based indicators of captured cells during imaging processes such as imaging cytometry.

In some embodiments, the filter membrane 120 is formed of a material selected to be mechanically and chemically stable as well as chemically and electrically inert. The filter membrane 120 includes a mechanical strength or rigidity to withstand pressure from fluid flow as the cell samples flow over and through the microfluidic chip. Advantageously, the filter membranes 120 described herein have sufficient structural integrity and rigidity to limit or avoid buckling, sagging, or breaking under pressure from the flow of fluid or gravitational forces. For example, filter materials can be selected that withstand pressures greater than or equal to 3 psi resulting from fluid flow over and/or through the filter membrane.

Additionally, the filter membrane 120 can be formed of a material having specific mechanical properties to withstand the insertion of a micromanipulator while harvesting, removing, and/or plucking cells of interest or cells not of interest from the filter membrane. For example, a micromanipulator may include a miniscule needle configured to pluck fragile cells captured in each through hole of the filter membrane. The insertion and removal of the needle in each through hole may exert an outward force onto the sidewalls of a given through hole, thus the filter membrane may be selected to withstand this force such that the filter membrane does not break nor is the through hole deformed.

Additionally, filter membranes with desired holes and configurations could be opaque or translucent for certain applications, made of materials such as silicon, for example. Filter membranes described herein can be generated or formed through specific chemical or electrochemical processes to desired thickness ranging from several microns to tens of microns or possibly hundreds of microns, followed by separative lift-off techniques, and then anodically bonded or attached through specific adhesive techniques to substrates that could be of several materials, or layers of materials, like silicon, organic polymers, glass, or plastics with different shapes and/or sizes.

As a result, filter membranes described herein can be used more than once to capture cells of interest in a sample or multiple portions of the same sample, representing a significant improvement over existing filter devices. For example, a first portion of a sample can be applied to the filter membrane 120, capturing a first subset of cells of interest in the first portion of the sample. In implementations described in detail below in which electrodes associated with a through hole can apply electrical forces to the through hole, an object captured in the through hole can be analyzed, determined to be an object not of interest, and then individually targeted and controlled in order to clear the through hole (for example, the object not of interest can be deformed in a particular direction to allow the object to pass through the through hole and out of the filter membrane, fragmented and/or destroyed so that the object no longer occupies the through hole, etc.). Subsequently, a second portion of a sample can be applied to the same filter membrane 120, capturing a second subset of cells of interest in through holes of the filter membrane 120 that are not occupied by an object (whether a cell of interest or some other undesired object). This process can be repeated until the entire sample has been applied to the same filter membrane 120, or until it is determined that a sufficient number of cells of interest have been captured in the filter membrane 120. Imaging of the filter membrane 120, manipulation of objects in the filter membrane, and other processes can take place at regular intervals or before the next sample portion is applied to the filter membrane. In some cases, at the end of this capture process, the microfluidic chip 100 will have a very high density of cells of interest in a single filter membrane 120, with each cell of interest isolated in a single through hole at a distinct, precisely-defined x, y location of the filter membrane 120. In one example, a monolayer of cells of interest is held in place on the side 112 of the substrate 110 by the filter membrane 120, and provides a unique platform from which to analyze, identify, and extract cells of interest from the microfluidic chip 100.

In some embodiments, the filter membrane 120 is formed of a material having hydrophilic properties, as opposed to hydrophobic properties. The hydrophilic properties of the filter membrane 120 permit the fluid sample to flow smoothly through the through holes. In some implementations, a surface on the first side of the filter membrane 120 is treated to obtain hydrophilic characteristics. In other implementations, the filter membrane 120 is formed of a material or materials having the desired hydrophilic characteristics. Advantageously, the hydrophilic properties of the filter membrane can prevent cells from clumping together as they flow through the filter membrane 120, thereby reducing the amount of pressure or force that is required to push the sample (and cells that are not of interest) through the through holes of the filter membrane 120. This reduction in the amount of pressure or force exerted on the filter membrane 120 during the capture process represents a significant improvement over existing filter systems, because embodiments of the filter membranes 120 described herein are less likely to puncture, bend, warp, bulge, or otherwise degrade during one or more capture processes, resulting in a longer life span of a single filter membrane 120 and the ability to use a single filter membrane 120 for multiple capture processes.

In some embodiments, the substrate 110 of an exemplary microfluidic chip 100 typically employs a solid or semi-solid substrate that may be planar in structure, i.e., substantially flat or having at least one flat surface. Suitable substrates may be fabricated from any one of a variety of materials, or combinations of materials. Often, the planar substrates are manufactured using solid substrates common in the fields of microfabrication, e.g., silica-based substrates, such as glass, quartz, silicon or polysilicon, as well as other known substrates, i.e., gallium arsenide, to ensure superior manufacturability and enhanced repeatedly of target dimensions. In the case of these substrates, common microfabrication techniques, such as photolithographic techniques, wet chemical etching, micromachining, i.e., drilling, milling, plasma etching, and the like, may be readily applied in the fabrication of microfluidic chips and substrates. An illustrative embodiment of such a fabrication process will be described in more detail below with reference to FIGS. 5 through 22B. Alternatively, polymeric substrate materials may be used to fabricate the devices of the present disclosure, including, for example, polydimethylsiloxanes (PDMS), polymethylmethacrylate (PMMA), polyurethane, polyvinylchloride (PVC), polystyrene, polysulfone, polycarbonate, and the like. In the case of such polymeric materials, injection molding or embossing methods may be used to form the substrates. In such cases, original molds may be fabricated using any of the above described materials and methods. The assembled microfluidic chips may be treated with plasma to alter surface wet-ability where desired post assembly or preferably treated first and then assembled.

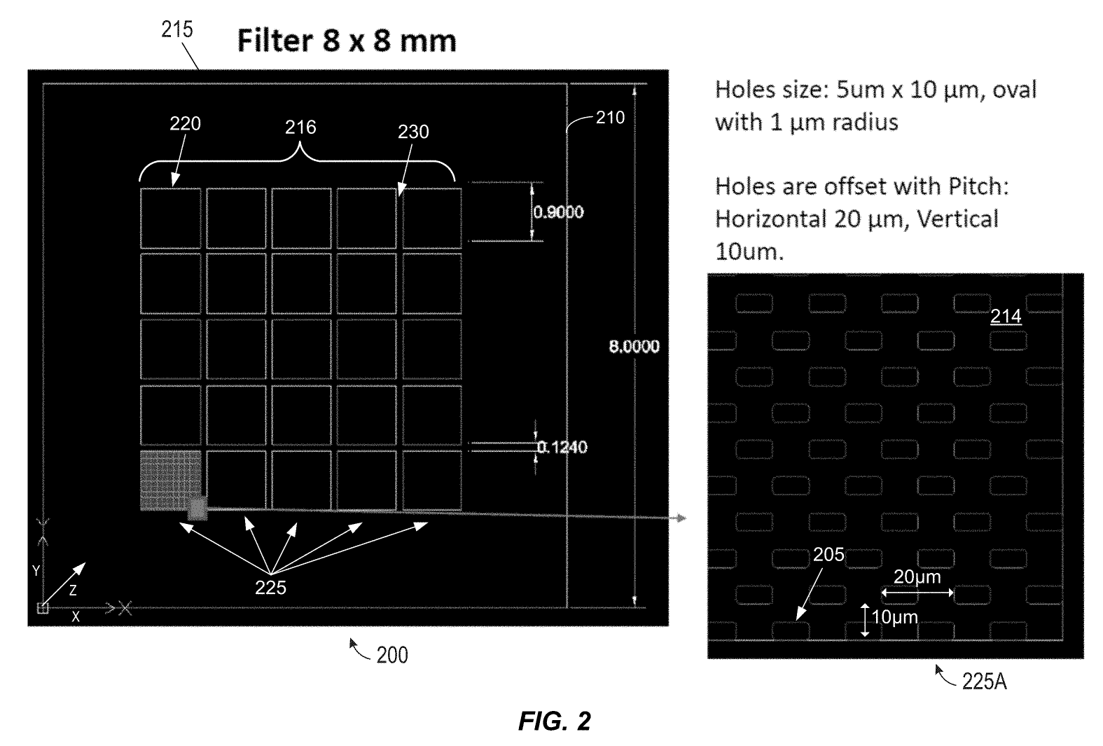

In accordance with methods described in detail below, the substrate 110 can be formed or manufactured to include multiple support vanes 130. In some embodiments (not illustrated), individual filter membranes 120 are held or disposed within hexagonal-shaped regions defined by vanes 130. In the embodiment illustrated in FIGS. 1A and 1B, the vanes 130 formed in the substrate 110 between the first side 112 and the second 114 define hexagonal-shaped filter regions 125 of a single filter membrane 120. For example, as illustrated in FIGS. 1A and 1B, the vanes 130 form a pattern of honeycomb-shaped cells 140. The filter membrane 120 disposed on the side 112 of the substrate 110 covers each honeycomb cell 140. Each honeycomb cell 140 (visible on side 114 of substrate 110 in FIG. 1A and visible through the filter membrane 120 in FIG. 1B) defines a hexagonal-shaped filter region 125 of the filter membrane 120. The pattern of cells formed by vanes 130 is not limited to the honeycomb pattern seen in FIG. 1A, however. For example, the vanes 130 can form a pattern of square cells (see FIG. 2), rectangular cells (not illustrated), or cells of any other suitable shape.

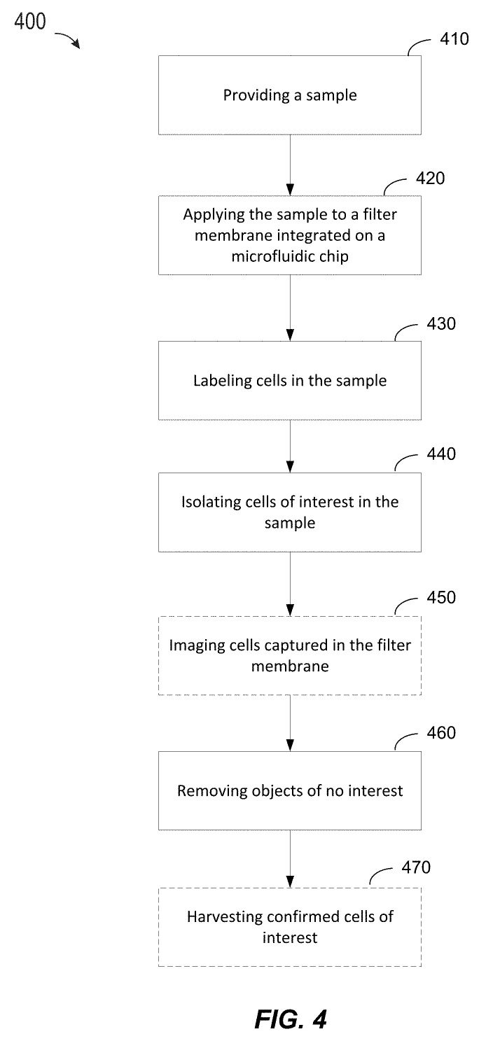

In some embodiments, vanes 130 are dimensioned and fabricated to provide support for the filter membrane 120. For example, the vanes 130 can support filter membrane 120 in a way that allows the filter membrane 120 to withstand a certain amount of pressure due to fluid flow. In the absence of vanes 130 in the substrate 110, the filter membrane 120 may sag, bend, or break due to the same amount of fluid being applied to a larger unsupported surface area of the filter membrane. In some aspects, vanes 130 advantageously provide further support and structural integrity for the filter membrane 120 than that provided by the sides 112, 114 of substrate 110, such that the middle of each filter membrane 120 does not sag, bend, or break due to pressures from fluid flow over and/or through the filter membrane 120. Further, vanes 130 forming honeycomb cells 140 can advantageously define a field of view (FOV) for imaging each hexagonal-shaped filter region 125 during an imaging cytometry process or other analysis as will be described below with reference to FIG. 4.

FIG. 2 illustrates an exemplary microfluidic chip 200 according to another embodiment. In this non-limiting example, the microfluidic chip 200 may be similar to the microfluidic chip 100 having a support layer and a filter layer, however the filter regions 225 are square in shape. In this case, the support layer includes a substrate 210 and the filter layer includes a filter membrane 220. The substrate 210 includes a frame-shaped exterior portion 215 and an interior portion 216 including vanes 230. The filter membrane 220 is positioned over and touching the vanes 230 in the interior portion 216. In this non-limiting example, the filter membrane is transparent such that the vanes 230 are visible through the filter membrane 220. The vanes 230 form a pattern of square-shaped filter regions. Other configurations are possible. In the example illustrated in FIG. 2, the vanes have a thickness of about 0.1240 millimeter measured along an x-axis and a y-axis of the microfluidic chip 200.

The vanes 230 define square-shaped filter regions 225 of filter membrane 220. It will be understood, however, that the microfluidic chip 200 can be designed to have filter regions 225 of any suitable shape (for example, hexagonal, square, or any other shape). Advantageously, features of the number, size, and shape of the filter regions 225 can be selected to maximize capture of a particular cell of interest, based on the intended application for the microfluidic chip 200.

The filter membrane 220 includes a first side 212 (not shown in FIG. 2) and an opposing, second side 214. The filter membrane 220 includes a plurality of through holes arranged in a regularly-repeating pattern, for example the close-up view of one filter region 225A illustrates a plurality of through holes 205 in the filter membrane 220. The through holes 205 extend between the first side 212 and the second side 214 of the filter membrane 220, thereby allowing objects to translocate through the filter membrane 220. The size, shape, and relative spacing of each through hole 205 can be specifically selected based on the cell of interest the filter membrane is designed to capture and retain, such that a single cell of interest is captured and retained in each through hole. The through holes can have openings that are generally rectangular in shape, generally circular in shape, or any other suitable shape.

One non-limiting advantage of filter membranes described herein is the ability to automatically create a monolayer of cells of interest as the sample flows through the filter membrane, which is not possible using a plating of the sample on a slide. Due to the specifically designed size, shape, and material properties of the through holes in the filter membranes, the filter membrane can be configured to prevent one potential cell of interest in the sample from obscuring, overlapping with, or lying on top of another potential cell of interest. As a result, imaging systems utilizing embodiments of the microfluidic chip described herein need not expend imaging resources, such as high resolution imaging resources, to determine where specific cell boundaries lie, to trace cell outlines to distinguish two closely-spaced cells from one another, or to ascertain if an object is actually two or more cells clumped together—activities that are typically required in conventional cell plating before a potential cell of interest is actually studied and confirmed to be a cell of interest.

In the non-limiting example depicted in FIG. 2, the substrate 210 includes an exterior portion 215 that is about 8 millimeters by about 8 millimeters measured along the x-axis and the y-axis of the microfluidic chip 200. The substrate 210 has a thickness of about 0.3 millimeter measured along a z-axis of the microfluidic chip 200. Other thicknesses are possible. The interior portion 216 in this example is about 5 millimeters by about 5 millimeters measured along the x-axis and the y-axis of the microfluidic chip 200.

In some cases, the square-shaped filter regions 225 defined by the vanes 230 can be referred to as the “active area” of the filter membrane 220. The areas of the filter membrane 220 that are disposed directly over and in contact with the vanes 230 are not considered “active areas” of the filter membrane 220 because the second openings of through holes in these areas may be partially or completely blocked by the vanes 230, such that fluid flow through these through holes is degraded or entirely obstructed. In this example implementation, the vanes 230 of microfluidic chip define 25 filter regions 225 arranged in a 5×5 grid. The vanes 230 can define fewer than 25 filter regions in the filter membrane 220, such as 9 filter regions (as with 9 filter regions arranged in 3×3 grid) or 16 filter regions (as with 16 filter regions arranged in a 4×4 grid). Some implementations include more than 25 filter regions, such as 64 or 100 filter regions. Other configurations are possible. In this non-liming example, each filter region 225 of the filter membrane 220 defines an active region that is about 0.9 millimeter by about 0.9 millimeter measured along the x-axis and the y-axis of the microfluidic chip 200.

As illustrated in the close-up view of one of the 25 filter regions, filter region 225A, the filter membrane 220 includes rectangular through holes, such as through hole 205, arranged in a regular, repeating pattern. However, it will be understood that any filter membrane described herein, not only that illustrated in FIG. 2, may be included in microfluidic chip 200 depending on the cells sought to be captured, imaged, and analyzed in a particular application. The rectangular through holes in filter membrane 220 are about 5 μm high (measured along the y-axis of the microfluidic chip 200) by about 10 μm long (measured along the x-axis of the microfluidic chip 200). Through holes having other shapes and dimensions are possible, for example, through holes can be about 4 μm high (measured along the y-axis of the microfluidic chip) by 8 μm long (measured along the x-axis of the microfluidic chip).

Each through hole 205 of filter membrane 220 is spatially separated, or offset, from other through holes by a horizontal pitch of about 20 μm (measured along the x-axis of the microfluidic chip) and a vertical pitch of about 10 μm (measured in the y-axis of the microfluidic chip). The offset dimensions can be advantageously selected to maximize the number of through holes in filter membrane 220 without sacrificing structural integrity of the filter membrane 220, thus maximizing the number of cells that can be captured in the filter membrane 220. In some embodiments, the through hole dimensions are kept at 50% of the pitch dimensions. As described above, these through hole dimensions and spacing are examples and other configurations are possible based on the specific size and shape of the objects (such as cells or microbeads) of interest to be isolated in the microfluidic chip 200. For example, each through hole can be offset from other through holes by a horizontal pitch of about 16 μm (measured along the x-axis of the filter membrane 420) and a vertical pitch of about 8 μm (measured along the y-axis of the filter membrane 420). The size, shape, and relative spacing of each through hole 205 in microfluidic chip 200 can be specifically selected based on the object of interest (such as a cell) the filter membrane 220 is designed to capture, such that a single object of interest is captured in each through hole 205.

In one example, a rectangular through hole 205 may be dimensioned to capture and retain a single RBC in the through hole based on the general disk-like shape of RBCs. In another example, a rectangular through hole 205 may be dimensioned to allow mature disk-shaped RBCs (such as maternal RBCs) to pass through the through hole 205, while a single fetal nucleated RBC is captured and retained in a single through hole 205 based on the spherical shape and slightly larger size of fetal nucleated RBCs. Thus, the characteristics and dimensions of each through hole may be specifically selected based on the shape and size of the cells of interest. Further, the density of through holes on a single filter membrane, and the relative position or arrangement of the through holes relative to each other, can be selected to optimize the number of cells of interest that are retained or “captured” in the filter membrane. Advantageously, the orientation of through holes in filter membranes described herein can be rotated, flipped, or shifted such as to maximize the number of through holes exposed to cells in the sample, thereby maximizing the number of cells of interest captured by the filter membrane.

In the embodiment illustrated in FIG. 2, the rectangular through holes 205 in this embodiment advantageously include rounded or chamfered corners. Rectangular through holes including rounded corners enhance fluid flow through the filter membrane 220. Without being bound to any particular theory, it is believed that the rounded or chamfered corners remove dead spots in the fluid flow through the through hole 220 that would ordinarily occur if the corners of the through hole included sharp angular edges. These sharp angular corners may cause the accumulation of fluid and/or cells within or around the corner. In this way, embodiments of through holes described herein can advantageously permit smooth flow of fluid through the filter. In some embodiments, the side walls of through hole extending through filter membrane can be advantageously angled or tapered (not shown) relative to the surface of the filter membrane. Without being bound to any particular theory, it is believed that tapered sidewalls permit cells of interest to more freely and consistently enter the through hole while also inhibiting the cell of interest from passing entirely through the through hole, thereby facilitating improved capture of the cell in the through hole.

In other embodiments, the through hole 205 may have an opening that is generally circular. The circular through holes may be specifically designed and configured to capture any desired cell, microbead, or other object based on known characteristics (such as, but not limited to, the size and morphology) of the sought after cell, microbead, or other object. By changing the shape and size of the through holes, multiple filter membranes can be designed and manufactured for the isolation of specifically sought after cells or objects. In one non-limiting example, a filter membrane is designed to include circular holes that are shaped and sized to capture specifically-identified bacterial cells of interest. In one non-limiting example, circular through holes have a diameter of about 10 μm. Other dimensions are possible. For example, circular through holes can have a diameter of about 5 μm or a diameter of about 7 μm. In one example implementation, circular through holes have a diameter of approximately 6.5 μm.

Microfluidic Chip with Filter Membrane Having Electrically-Controllable Through Holes

Embodiments of integrated microfluidic devices including a plurality of electrodes associated with each through hole of a plurality of through holes will now be described. Each plurality of electrodes is precisely aligned with its respective through hole and configured to apply electrical forces to an object captured in the respective through hole. In the following embodiments, two electrodes are aligned with a single through hole and configured to apply electrical forces to an object captured in the through hole, but the number of electrodes associated with and configured to apply an electrical force to a through hole can vary. In some embodiments, for example, three, four, or more electrodes are aligned with a single through hole and configured to apply electrical forces to an object captured in the through hole. As used herein, the terms “electrode/through hole pair” refers to a through hole in a filter membrane and the plurality of electrodes associated with and configured to apply an electrical force to that through hole (and any object that may be captured in that through hole). Each electrode/through hole pair also includes electrical conducting lines configured to communicate electrical signals to the plurality of electrodes from a controller. Each plurality of electrodes is associated with a single through hole having a distinct, precisely-defined location in the filter membrane, such that the plurality of electrodes associated with each through hole also has a distinct, precisely-defined location in the filter membrane. This enables precise control of electrical signals applied to each electrode associated with a single through hole independent of other electrodes associated with other through holes in the filter membrane. In this way, an electrical signal, e.g., a voltage bias, can be applied across each through hole and the electrical signal can be independently controlled for each through hole.

In instances where a through hole has captured a cell, microbead, or other object (whether it be an object of interest or an object not of interest), the electrical signal (e.g., voltage bias) that is applied to the through hole is also applied to the object, such as a cell, captured in the through hole. Embodiments of microfluidic chips described herein can apply a voltage bias to the captured cell, and precisely control the magnitude of that voltage bias applied to that captured cell, such that the cell associated with a specific through hole can be manipulated, e.g., electrical forces attract the cell or a portion of the cell in a particular direction, electrical forces repel the cell or a portion of the cell in a particular direction, electrical forces fragment the cell, or electrical forces destroy the cell based on the voltage bias applied to the one or more electrodes associated with the through hole. In one example implementation, an object captured in a through hole is determined to be an object not of interest. The plurality of electrodes associated with the through hole can apply a voltage bias to deform the object (for example, stretch, lengthen, or change the cross sectional diameter of a portion of the object), allowing the entire object to pass through the through hole and out of the filter membrane, thereby clearing the through hole entirely. Alternatively, the plurality of electrodes associated with the through hole can apply a voltage bias and fragment the object in such a way that some or all of the fragments pass through the through hole and out of the filter membrane, thereby clearing the through hole partially or entirely. Microfluidic chips described herein can control the voltage bias applied to each electrode/through hole pair independent of other electrode/through hole pairs, thereby enhancing cell sorting and filtration in a single device, wherein selected cells captured in the filter membrane are targeted for removal from the filter membrane while other cells captured in the filter membrane remain unaffected.

The significance of the hydrodynamic aspects of the filter membrane manifested in the tapered sidewall angles comes into effect here, specifically with the fact that applied voltages for the electrode functionality would not have to be unnecessarily applied for long time intervals to captured cells of interest for continued retention. Localized resistive heating emitted by the electrodes is a possible outcome of continued applied voltages to conductive electrodes, and localized heating could be detrimental to good, desired, and sought after rare cells of choice. Hence, the hydrodynamic trapping effect of the filter holes minimizes the need to unnecessarily or excessively activate the electrodes beyond their initial guiding effects (i.e. through exerting attractive or repulsive forces) towards the filter through holes, which then act as an effective capturing grid without any further electrical requirements from the through holes neighboring electrodes. This characteristic of filter systems and methods described herein results in minimizing or eliminating any potential thermal damage effects on captured desired cells.

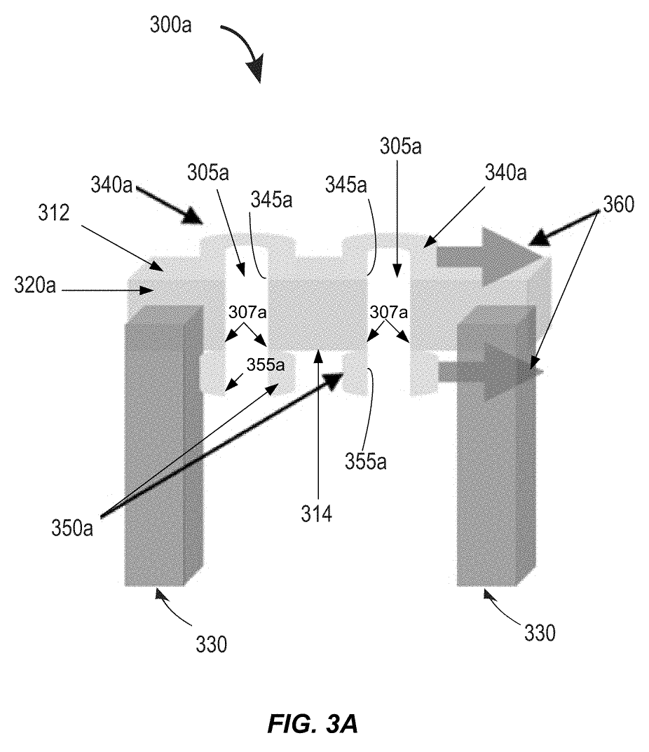

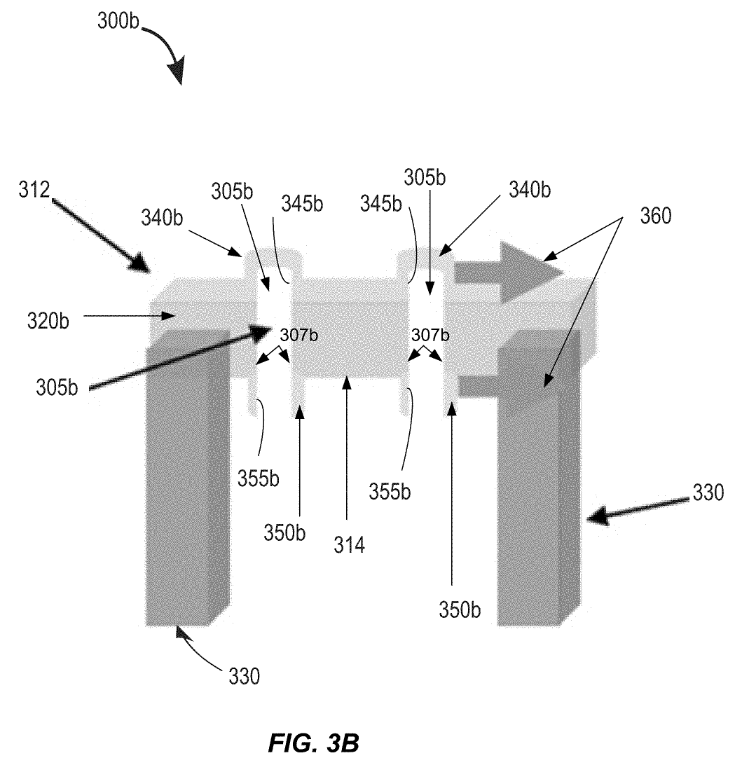

FIGS. 3A and 3B illustrate cross-sectional side views of exemplary microfluidic chips 300a and 300b having filter membranes with electrically-controllable through holes. FIGS. 3A and 3B are schematic representations and are not drawn to scale. Although filter-membranes having electrically-controllable through holes will be described with reference to the microfluidic chips 300a and 300b, it will be understand that the features of microfluidic chips 300a and 300b can be implemented in microfluidic chips described in accordance with the present disclosure, including but not limited to the microfluidic chip 100 and the microfluidic chip 200 described above reference to FIGS. 1A, 1B, and 2.

The microfluidic chips 300a and 300b include through holes 305a and 305b, respectively, having a generally circular cross-section as measured along the x-axis and the y-axis of the microfluidic chips. The microfluidic chips 300a and 300b include a plurality of electrode/through hole pairs. More specifically, the microfluidic chip 300a includes a plurality to first electrodes 340a, a plurality of second electrodes 350a, and a plurality of electrical connections 360 coupling the first electrodes 340a and the second electrodes 350a to a controller (not shown). The microfluidic chip 300b includes a plurality of first electrodes 340b, a plurality of second electrodes 350b, and a plurality of electrical connections 360 coupling the first electrodes 340b and the second electrodes 350b to a controller (not shown). Each first and second electrode (e.g., first electrode 340a and second electrode 350a in microfluidic chip 300a and first electrode 340b and second electrode 350b in microfluidic chip 300b) is associated with a single through hole (e.g., through hole 305a in microfluidic chip 300a and through hole 305b in microfluidic chip 300b), thereby defining an electrode/through hole pair. By precisely aligning and positioning the electrodes relative to the through hole, the electrode/through hole pairs enable precise and independent control of a voltage bias applied to the through hole and any contents therein. The support layer in the microfluidic chips 300a and 300b includes a substrate having vanes 330, as detailed below with reference to FIGS. 5 through 22B, and the filter layer includes a filter membrane (filter membrane 320a in microfluidic chip 300a and filter membrane 320b in microfluidic chip 300b). In these example implementations, the filter membranes in microfluidic chip 300a and microfluidic chip 300b are positioned over and touching the vanes 330.

In this non-limiting example, the filter membrane is transparent such that the vanes 330 are visible through the filter membrane. The microfluidic chips 300a and 300b are substantially the same, however, the through holes 305a and 305b have different dimensions and orientations, and the electrodes 340a and 350a are of a different shape and configuration than electrodes 340b and 350b, as will be described in reference to an exemplary method of fabrication detailed in reference to FIGS. 5 through 22B. For purposes of illustrating features that microfluidic chip 300a and microfluidic chip 300b have in common, the following description will illustrate certain features of microfluidic chip 300a with reference to FIG. 3A, but it will be understood that the described aspects of microfluidic chip 300a also apply to microfluidic chip 300b.

Referring now to FIG. 3A, the through holes 305a include sidewalls 307a extending between a first side 312 and a second side 314 of the filter membrane 320, thereby allowing objects to translocate through the filter membrane 320. The first side 312 and second side 314 also include first electrodes 340a and second electrodes 350a disposed on each side, respectively, as illustrated. First electrodes 340a include side walls 345a extending between a top surface of the first electrode and a bottom surface of the first electrode (adjacent to the first side 312 of the filter membrane 320). The side walls 345a of the first electrode 340a are aligned with the side walls 307a of each respective through hole 305a, thereby allowing objects to translocate through the first electrode 340a and into its respective through hole 305a. Once in through hole 305a, the object may translocate through the filter membrane 320 or be captured and retained in the through hole 305a, as described above. Second electrodes 350a also include side walls 355a extending between a top surface of the second electrode (adjacent to the second side 314 of the filter membrane 320) and a bottom surface of the second electrode. The side walls 355a of the second electrode 350a are also aligned with the side walls 307a of its respective through hole 305a, thereby allowing objects to translocate through the through hole 305a and then through the second electrode 350a. In one non-limiting embodiment, the features of an angled sidewall of a through hole serve dual functions: one being a physical hydrodynamic trap preventing captured cells or beads of further lateral or directional movement, and the other being a filtering or isolating membrane. If the through holes do not include tapered side walls, the through holes may only serve to prevent certain cells from flowing through the filtering membrane, but would not serve as a hydrodynamic trap, or capturing grid, for cells or beads of targeted size, thus retaining and immobilizing them within, or partially within, the through holes. In one non-limiting example of a circular through hole, the thickness of the membrane and the angle of the through hole side walls determine the capturing and immobilizing characteristics of the filter membrane, and the smallest diameter, at the bottom of the through hole, determines its filtering or isolating properties. Similar effects can be achieved in non-circular through holes by independently selecting the angle of the tapered side wall and the smallest dimension (measured along the x-axis and the y-axis) of the through hole.

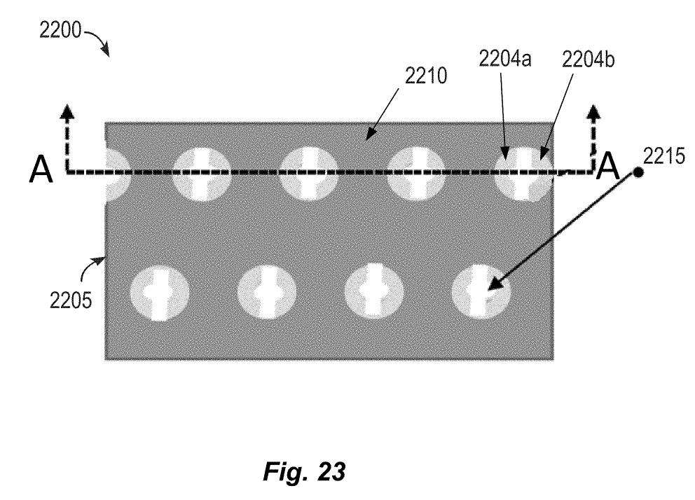

While the embodiments illustrated in FIGS. 3A and 3B depict a first electrode disposed on a first side of the filter membrane (in these non-limiting examples, positioned in direct contact with a top surface of the filter membrane) and a second electrode disposed on to a second, opposing side of the filter membrane (in these non-limiting examples, positioned in direct contact with an opposing, bottom surface of the filter membrane), other configurations are possible. For example, the first electrode and the second electrode can both be disposed on the same single side of the filter membrane, as described with reference to FIGS. 23 through 25I. Embodiments of microfluidic chips having first and second electrodes disposed on the same side of the filter membrane can fragment and/or destroy cells captured in the corresponding through hole.

Microfluidic chips described herein can manipulate an object captured in a particular through hole by applying a voltage bias to the plurality of electrodes associated with the through hole, offering an enhanced and selective filtration method. Manipulating the object in the through hole can include changing the physical dimension of the object or part of the object (for example, stretching, deforming, or lengthening the object or part of the object) and/or discarding the object from the filter membrane (for example, fragmenting or destroying the object). The microfluidic chip 300a includes a plurality of through holes 305a that each have a distinct and precisely-defined location within the filter membrane 320a. Each through hole 305a is associated with a first electrode 340a and a second electrode 350a, together defining an electrode/through hole pair. Also, each first electrode 340a and each second electrode 350a is electrically connected to an electrical connection 360, thereby allowing a voltage bias to be applied to the first and second electrodes 340a and 350a associated with a selected through hole, independent of any other electrode/through hole pair in the device. Advantageously, an object located in a particular through hole at a distinct, precisely-defined location in the filter membrane can be selectively manipulated by independently controlling the first and second electrodes 340a and 350a associated with the through hole.

In some embodiments, there is a one electrical connection 360 for both the first electrode 340a and the second electrode 350a in each electrode/through hole pair. Electrical connections 360 allow the first and second electrodes 340a and 350a associated with a selected through hole to be independently controlled. Specifically, a voltage bias can be applied across the first and second electrodes 340a and 350a in each electrode/through hole pair to selectively attract, repel, destroy, fragment, distort, or otherwise manipulate the objects or cells captured in each through hole 305. The voltage across the electrodes can be manipulated or otherwise adjusted, and depending on the voltage applied across the first and second electrodes and the cell (or other object) captured in the through hole, the cell may be selectively removed from the through hole or selectively retained in the through hole. Without being bound to any particular theory, it is believed that adjusting the electrical bias to a precisely controlled bias magnitude will enable specifically sought after physical manipulation of the cells or objects captured in a given through hole. For example, at a first magnitude of voltage bias electrical forces can attract or repel the object (or a portion of the object) in a particular direction, thereby clearing the through hole of the object. At a second magnitude of voltage bias, electrical forces fragment the object into pieces which may then be allowed to pass through the through hole and out of the filter membrane, thereby clearing the previously-occupied through hole partially or entirely. At a third magnitude of voltage bias, electrical forces may destroy or lyse the object contained in the through hole, thereby clearing the through hole of captured object. At a fourth magnitude of voltage bias, electrical forces may elongate or otherwise manipulate the physical shape of the object, thereby permitting the object to pass through the through and clearing the through hole of the object. Depending on the characteristics of the object sought to be manipulated with a voltage bias, the first, second, third, and fourth magnitude of voltage bias may be selected to be the same voltage bias but applied for different lengths of time, or the first, second, third, and fourth magnitude of voltage bias may be selected to be different. The preceding are presented as examples of object manipulation due to electrical forces, and other forms of clearing the through hole of the object based on adjusting the voltage bias are possible. An exemplary process of using a microfluidic chip having first and second electrodes, similar to those described with reference to FIG. 3, will now be described with reference to FIG. 4. Above-described advantages associated with minimizing detrimental heating effects of electrodes on desired cells are applicable in these examples.

FIG. 4 is a flow diagram illustrating one exemplary process 400 to obtain cells of interest (such as fetal nucleated RBCs, trophoblasts, or another cell of interest) from a sample using microfluidic chips described herein. As illustrated in FIG. 4, the method 400 can include one or more functions, operations or actions as illustrated by one or more operations 410-470.

It is noted that the examples may be described as a process, which is depicted as a flowchart, a flow diagram, a finite state diagram, a structure diagram, or a block diagram. Although a flowchart may describe the operations as a sequential process, many of the operations can be performed in parallel, or concurrently, and the process can be repeated. In addition, the order of the operations may be re-arranged. A process is terminated when its operations are completed. A process may correspond to a method, a function, a procedure, a subroutine, a subprogram, etc. When a process corresponds to a software function, its termination corresponds to a return of the function to the calling function or the main function.

Embodiments of filter membranes described herein can separate, or filter, fetal nucleated red blood cells (RBCs) from a maternal blood sample containing mature (non-nucleated) maternal RBCs and fetal nucleated RBCs. Fetal nucleated RBCs circulating in the maternal blood are extremely rare, with some estimates as low as 1 in a 10 million. Mature human RBCs are oval biconcave disks and generally lack a cell nucleus. In contrast, fetal nucleated RBCs are slightly larger than mature maternal RBCs and generally spherical rather than disk-shaped. Embodiments of the morphology-based selection filters described herein include through holes with a specific shape, size, and arrangement such that most or all of the mature red blood cells (RBCs) in a sample pass through the through holes in the filter while some, most, or all of the fetal nucleated RBCs are retained or “captured” in the through holes. However, due to variations in the morphology of the RBCs, some maternal RBCs may also be captured in through holes in the filter even though they are not cells of interest. For illustrative purposes, the following description provides methods of non-invasive prenatal testing (NIPT) and for isolation, identification, and harvesting of fetal nucleated RBCs for non-invasive prenatal diagnosis. While the exemplary embodiment disclosed herein may describe isolation of fetal nucleated RBCs from a maternal blood sample for non-invasive prenatal diagnosis, the skilled artisan will understand that the principles and concepts of the methods and devices described herein are applicable beyond NIPT. Accordingly, embodiments of the methods and systems described herein can be used in numerous applications, including but not limited to NIPT. For example, methods and devices disclosed herein may be configured for the isolation of microbeads, tumor cells for oncology, or any other pathological condition where cells of one kind can be differentiated from cells of another kind based on size, morphology, nuclear staining, and/or biomarker identification.

Embodiments of methods and devices disclosed herein can obtain cells of interest using morphology-based isolation, affinity and/or biomarker-based detection and identification combined with electrical bias-based cell sorting and filtration. By combining these processes on an integrated microfluidic chip in accordance with the embodiments described herein, the method 400 resolves long-standing challenges associated with isolating specific cells of interest from a sample of cells. Unlike fluorescence activated cell sorting (“FACS”) utilized in flow cytometry, embodiments disclosed with reference to method 400 are visualization-based methods similar to imaging cytometry that are performed on a microscope platform, but advantageously address drawbacks associated with prior imaging cytometry-based systems and methods. Method 400 can be partially or fully automated which adds another benefit to embodiments described in the current disclosure.

Cytometry, including flow cytometry and imaging cytometry, is the measurement and/or identification of cell characteristics. Cytometry methodologies are configured to measure any of a number of parameters, including for example cell size, cell count, cell shape and structure, cell cycle phase, DNA content, and the existence or absence of specific proteins on the cell surface or within the cell. There are many applications in which the different cytometry methods can be used. For example, cytometry can be used in characterizing and counting blood cells in a sample of blood, cell biology research, and medical diagnostics to characterize cells in pathological diseases (e.g., cancer and AIDS). Imaging cytometry is one type of cytometry that operates by statically imaging a large number of cells using optical microscopy. Prior to analysis, cells can be stained to enhance contrast or detect specific molecules by labeling these with nuclear stains, biomarkers, and/or fluorescent dyes.

Embodiments of microfluidic chips described herein can advantageously be used in imaging cytometry to develop a representation (for example, obtain an image or take a picture) of all of the captured cells in a specific area of interest in a single image. In one non-limiting aspect, the specific area of interest is one region of a plurality of regions of a single filter membrane of a microfluidic chip. In another non-limiting aspect, the specific area of interest is one filter membrane arranged in a microfluidic chip including one single filter membrane. In another example, the specific area of interest is one filter membrane of a plurality of filter membranes arranged in a microfluidic chip. Due to the precisely-defined and repeating grid pattern of through holes in the filter membrane(s), the exact position of each captured and hydrodynamically retained cell can be identified using the unique position of its corresponding through hole in the filter membrane(s). In one embodiment, capturing and simultaneously positioning of cells of interest in this way allows the cells of interest to be analyzed for verification that the captured cells are, in fact, cells of interest. For example, where the cell samples have been stained and/or labeled with nuclear stain, specific biomarkers, and/or fluorescent dyes used to identify user-defined characteristics of the cells, captured cells with those characteristics may be readily identified and their position can be easily returned to for subsequent, more detailed analysis of a captured cell or for manipulation or extraction of a captured cell. In another embodiment, the capturing and simultaneous positioning of cells of interest in this way allows the cells of interest to be subject to steps of cell lysis and DNA extraction for downstream genetic analysis. For example, the captured, isolated, and sorted cells of interest can be assessed for a nucleotide sequence of nucleic acid molecules or expression of a gene. Additionally, as described above with reference to FIG. 3, a specific object captured in a through hole in the filter membrane that is determined to not be a cell of interest can be advantageously manipulated (for example, distorted, stretched, lengthened, or fragmented) to remove the object from the through hole, thereby clearing the through hole to receive another object (such as a cell of interest) during subsequent sample filtration steps. The process of applying a sample to the filter, analyzing the objects that were captured in the filter membrane during the filtration, and removing specific objects that are identified as not of interest by targeting those specific objects for manipulation and/or destruction can be repeated multiple times, resulting in a large density of objects of interest being captured on the filter membrane for later harvesting and downstream genetic and or diagnostic testing.

Embodiments of filters described herein can advantageously be used to distinguish captured cells of interest from captured cells that are not of interest using biomarkers specific to the cells of interest (in this non-limiting example, fetal nucleated red blood cells (fnRBCs)). For example, before or after the sample is run through the filter and cells are captured in the filter membrane(s), the cells can be stained and/or labeled with nuclear stain, specific biomarkers, and/or fluorescent dyes for positive or negative selection of a subset of the captured cells (for example, positive selection of captured fetal nucleated RBCs and negative selection of captured cells that are not fetal nucleated RBCs). Embodiments of filters described herein can advantageously use the identification of cells not of interest to remove or otherwise manipulate the cells not of interest. For example, precise electrical manipulation of electrodes associated with the through holes may be performed based on said criteria. Through holes identified to have captured cells not of interest may be independently manipulated to generate a voltage bias that removes or destroys specifically-identified unwanted cells.

As used herein, “microscope platform” refers to a system and/or device configured to perform imaging of cells. In one aspect, a microscope platform includes an epifluorescence microscope. The microscope platform may include an imaging device configured with an adjustable or multiple magnification objective (e.g., 10x, 40x, 60x, etc.), and an image sensor configured to obtain an image based on the light received through an imaging device lens. In some embodiments, the imaging device includes a field-of-view (“FOV”) that is configured to match the size and shape of at least one region of a filter membrane of the microfluidic chip as defined by vanes of a substrate that support the filter membrane. In some embodiments, the microscope platform may be configured to scan along a microfluidic filter membrane including a plurality of filter regions and obtain at least one image of each filter region, where the dimension of each filter region corresponds to the FOV of the imaging device.

Referring to now to FIG. 4, method 400 can begin at operation 410, “Providing a sample.” Operation 410 can be followed by operation 420, “Applying the sample to a filter membrane integrated on a microfluidic chip.” Operation 420 can be followed by operation 430, “Labeling cells in the sample.” Operation 430 can be followed by operation 440, “Isolating cells of interest in the sample.” In some cases, operation 420 and operation 440 are performed simultaneously. Operation 440 can be followed by an optional operation 450, “Imaging cells captured in the filter membrane.” Operation 450 can be followed by an operation 460, “Removing objects of no interest.” The method next can move to optional operation 470, “Harvesting confirmed cells of interest.”

At operation 410, “Providing a sample,” a sample containing cells of interest may be provided. For example, maternal samples containing one or more fetal nucleated cells, such as red blood cells, can be obtained from human pregnant mothers using standard blood draw. The maternal sample can be taken during the first trimester (about the first three months of pregnancy), the 2nd trimester (about months 4-6 of pregnancy), or the third trimester (about months 7-9 of pregnancy). In some embodiments, a blood sample is obtained from a pregnant human mother even after a pregnancy has terminated. Typically, the sample obtained is a blood sample.

At operation 420, “Applying the sample to a filter membrane integrated on a microfluidic chip,” embodiments of microfluidic chips having filter membranes described herein that are suitable to select fetal nucleated blood cells may be used. In some embodiments, the microfluidic chip and filter membrane used in this non-limiting example are substantially similar to the microfluidic chips depicted in FIGS. 1A through 3B. Accordingly, in some embodiments, fetal nucleated RBCs may be captured when mature RBCs pass through filter holes having a size and/or shape that allow mature RBCs to pass through, but not fetal nucleated RBCs.

In some embodiments, a filter membrane may be coated with a binding moiety or affinity molecule that selectively binds fetal nucleated cells, such as fetal nucleated RBCs. For example, an antibody that specifically binds to fetal nucleated RBCs may be used to coat the filter membrane, so that fetal nucleated RBCs are retained while the mature RBCs pass through the filter membrane.

In some embodiments, a sample applied to a filter membrane at operation 420, can be dominated (>50%) by cells not of interest (e.g., nucleated maternal red blood cells). In some cases, the nucleated fetal cells of a sample applied to the filter membrane make up at least 2, 3, 4, 5, 10, 20, 30, 40, 50, 60, 70, 80, 90 or 95% of all cells in the sample. In some embodiments, the use of embodiments of microfluidic chips disclosed herein have removed at least 50, 60, 70, 80, 85, 90, 91, 92, 93, 94, 95, 96, 97, 98, 99, 99.5, 99.6, 99.7, 99.8 or 99.9% of all unwanted analytes (e.g., maternal cells such as platelets and leukocytes, mature RBCs) from a sample.

At operation 430, “Labeling cells in the sample,” cells may be labeled, directly or indirectly, with a dye in a staining process. Any fluorescent dye that is used in fluorescence microscopy can be used. For example, the nucleated fetal RBCs may be labeled, directly or indirectly, with a dye indicative of certain characteristics of the cell. In some embodiments, the labeling procedure of operation 430 may be performed prior to, during, or after operation 420. In some embodiments, a dye that stains DNA, such as Acridine orange (AO), ethidium bromide, hematoxylin, Nile blue, Hoechst, Safranin, or DAPI, may be used. In some embodiments, a cell type-specific dye, for example, a dye that specifically labels a fetal cell or a non-fetal cell, may be used. The cell type-specific dye may be used to label the cells directly or indirectly, for example, through a cell type-specific antibody. The labeling strategy involved may be sequentially carried out or simultaneously carried out.

Any of a variety of fluorescent molecules or dyes can be used to label cells in methods provided herein, including, but not limited to, Alexa Fluor 350, AMCA, Alexa Fluor 488, Fluorescein isothiocyanate (FITC), GFP, RFP, YFP, BFP, CFSE, CFDA-SE, DyLight 288, SpectrumGreen, Alexa Fluor 532, Rhodamine, Rhodamine 6G, Alexa Fluor 546, Cy3 dye, tetramethylrhodamine (TRITC), SpectrumOrange, Alexa Fluor 555, Alexa Fluor 568, Lissamine rhodamine B dye, Alexa Fluor 594, Texas Red dye, SpectrumRed, Alexa Fluor 647, Cy5 dye, Alexa Fluor 660, Cy5.5 dye, Alexa Fluor 680, Phycoerythrin (PE), Propidium iodide (PI), Peridinin chlorophyll protein (PerCP), PE-Alexa Fluor 700, PE-Cy5 (TRI-COLOR), PE-Alexa Fluor 750, PE-Cy7, APC, APC-Cy7, Draq-5, Pacific Orange, Amine Aqua, Pacific Blue, Alexa Fluor 405, Alexa Fluor 430, Alexa Fluor 500, Alexa Fluor 514, Alexa Fluor-555, Alexa fluor-568, Alexa Fluor-610, Alexa Fluor-633, DyLight 405, DyLight 488, DyLight 549, DyLight 594, DyLight 633, DyLight 649, DyLight 680, DyLight 750, or DyLight 800. Such fluorescent molecules or dyes may produce a corresponding light signature or spectrum upon being illuminated or excited by a light source having a particularly corresponding wavelength of light. Thus, using specific fluorescent molecules or dyes may produce an indicator of a particular nucleic acid, antibody or antibody-based fragment probe present on or within a fetal cell.

In some embodiments fetal biomarkers can be used to label one or more fetal cells at operation 430 of FIG. 4. For example, this can be performed by distinguishing between fetal and maternal cells based on relative expression of a gene (e.g., DYS1, DYZ, CD-71, MMP14) that is differentially expressed during fetal development. In one embodiment of the present disclosure, detection of transcript or protein expression of one or more genes including, MMP14, CD71, GPA, EGFR, CD36, CD34, HbF, HAE 9, FB3-2, H3-3, erythropoietin receptor, HBE, AFP, APOC3, SERPINC1, AMBP, CPB2, ITIH1, APOH, HPX, beta-hCG, AHSG, APOB, J42-4-d, 2,3-biophosphoglycerate (BPG), Carbonic anhydrase (CA), or Thymidine kinase (TK), is used to enrich, purify, enumerate, identify, detect, or distinguish a fetal cell. The expression can include a transcript expressed from these genes or a protein. In one embodiment of the present disclosure, expression of one or more genes including MMP14, CD71, GPA, EGFR, CD36, CD34, HbF, HAE 9, FB3-2, H3-3, erythropoietin receptor, HBE, AFP, AHSG, J42-4-d, BPG, CA, or TK, is used to identify, purify, enrich, or enumerate a fetal nucleated cell such as a fetal nucleated RBC.

In another embodiment of the present disclosure, fetal cells known as trophoblasts are a cell of interest that is isolated using filters described herein. Biomarkers specific to trophoblasts can be labeled and used to distinguish the fetal trophoblast cells (that are captured in the filter and are objects of interest) from maternal cells (that are also captured in the filter but are not objects of interest). Biomarkers that can be used to label, identify, detect, or distinguish a fetal trophoblast cell include (but are not limited to) cytokeratin 5, 6, 7, 8, 10, 13, 14, 18, 19; CD147, CD47, CD105, CD141, CD9, HAI-1, CD133, HLA-G, human placental lactogen, PAI-1, and IL-35. Other biomarkers that are not specific to fetal trophoblast cells but that can be used to label, identify, detect, or distinguish fetal cells of interest from maternal cells that are not of interest, include (but are not limited to) CD68, CD105, placental alkaline phosphatase (PLAP), NDOG, GB25, β-hCG, and 3b-hydroxy-5-ene steroid dehydrogenase. The above-referenced list of biomarkers provides examples of suitable biomarkers for labeling, identifying, detecting, or distinguishing a fetal cell from a maternal cell and is not intended to limit methods and devices described herein, which can capture and identify any cell of interest that is subject to filtration, whether or not the cell of interest has biomarkers that are used to distinguish the cell of interest captured in the filter from another object that is also captured in the filter but is not a cell of interest.

At operation 440, “Isolating cells of interest in the sample,” cells of interest such as fetal cells may be isolated using embodiments of microfluidic chips and filter membranes described herein with reference to FIGS. 1A through 3B. Isolating cells of interest can include positioning a single cell of interest at a distinct, precisely-defined location, such as a single through hole, in a filter membrane. As described above, each fetal nucleated RBC may be isolated from other cells in the sample (other fetal nucleated RBCs, non-nucleated fetal cells, maternal cells, etc.) when the fetal nucleated RBC is retained in a single through hole of the filter membrane while other cells that are not of interest (such as mature maternal RBCs) pass through the through holes of the filter membrane and are not retained in the filter membrane. Accordingly, the isolation operation 440 may be performed at the same time as operation 420.

In operation 450, “Imaging cells captured in the filter membrane” the cells captured in the filter at operation 420 and/or operation 440 are imaged for further analysis and genetic testing downstream. In some embodiments, imaging at operation 450 also includes imaging each filter region of a plurality of filter regions of a filter membrane using a microscope platform with a field of view (FOV) that matches the dimensions of a single filter region as defined by vanes of a substrate in the microfluidic chip, as described with reference to FIGS. 1A, 1B, and 2.

In some embodiments, cell samples are labeled or stained with fluorophores, fluorescent chemical compounds that can re-emit light upon light excitation. Cells samples can be labeled or stained with multiple kinds of fluorophores, each kind designed to emit a specific color of light upon light excitation. Embodiments of the microscope platform include an illumination source configured to illuminate fluorescently-stained cells in a filter with light of a specific wavelength which is absorbed by the fluorophores, causing them to emit light of longer wavelengths (i.e., of a different color than the absorbed light). The specific wavelength can be selected based on the nuclear staining and/or biomarker identification used to fluorescently stain the cell sample. In some embodiments, the microscope platform further includes a detector or a sensor configured to detect the spectral emission characteristics of the fluorophore used to label the fluorescently-stained cell. The distribution of a single fluorophore (color) can be imaged by the microscope platform. Multi-color images of several kinds of fluorophores can be developed using several single-color images. In one embodiment, the microscope platform is configured to have multiple illumination sources or modify the illumination of the captured cells to cause fluorescence of multiple different dyes.

At operation 460, “Removing objects of no interest,” cells and/or other objects that are captured in the filter membrane but that are not of interest are selectively and precisely removed from the filter membrane using embodiments of microfluidic chips and filter membranes described herein with reference to FIGS. 3A and 3B. Where captured objects are confirmed to be objects that are not of interest in either operation 440 or operation 450, operation 460 may be performed to destroy, fragment, or otherwise remove the captured object from its respective through hole. Once an object of no interest is removed, the through hole is now cleared of unwanted objects, thereby permitting an object of interest (such as, for example, a cell of interest) to be captured in that through hole. For example, at operation 440, cells are positioned at distinct, precisely-defined locations, such as a single through hole, in a filter membrane. As described above, each fetal nucleated RBC may be isolated from other cells in the sample when the fetal nucleated RBC is retained in a single through hole of the filter membrane. However, some of through holes of the filter membrane may capture other cells that are not of interest (such as mature maternal RBCs), due to of variations in cell shape or size or variations in through holes resulting from fabrication imperfections. Thus, some of the through holes contain cells of interest while some number of through holes contain cells or other objects that are not of interest following operation 440.

In a non-limiting embodiment, the microfluidic chip and filter membrane used to remove objects that are not of interest are substantially similar to the microfluidic chips depicted in FIGS. 3A and 3B. A voltage bias can be applied to electrodes associated with each through hole by selectively and independently applying a voltage difference across the electrodes to selectively attract, repel, destroy, fragment, and/or otherwise remove the objects that are not of interest. As described above, some of the through holes may contain cells not of interest (e.g., mature material RBCs or other objects that are not fetal nucleated RBCs) as identified in the operations 430 and/or 450. The distinct and precisely-defined location of each object not of interest can be readily determined based on the location of the through hole within the filter membrane. A voltage bias can be applied to these specific, identified objects by applying a voltage difference across the electrodes. This will impose a voltage bias to the object located in the through hole corresponding to the electrode/through hole pair, allowing for electrical and/or physical manipulation of one specific object without manipulating other objects (such as cells of interest) captured in nearby through holes.

Through manipulation and control of the electrical bias applied to the electrode/through hole pair, the object located in the through hole may be physically altered. In some embodiments where the retained object is a cell that is not of interest, the cell contained in the through hole is fragmented or broken into multiple pieces. The cell fragments may then pass through the through hole, thereby removing the cell that is not of interest from the filter membrane. In another embodiment, the attractive or repulsive forces exerted on the cell due to the voltage difference may pull or push the cell out of or through the through hole, thereby removing the unwanted cell from the filter membrane. In still another embodiment, the applied voltage difference damages the cell structure to such an extent that the cell is lysed within the through hole. In each instance, the electrical bias applied to the electrode/through hole pair can be manipulated and controlled to remove and/or discard the identified unwanted cell from the filter membrane. Thus, the distinct, precisely-defined position of each electrode/through hole pair within the microfluidic chip enables enhanced and automated manipulation, identification, and removal of captured objects that are not of interest, as well as identification and removal of cells that are of interest. While the preceding description was made with reference to manipulating, via a voltage bias, cells of no interest, it will be understood that the same process may be applicable to cells of interest based on the needs of the particular application for which the microfluidic chip is intended. Above-described advantages associated with hydrodynamic capturing and retaining effects of tapered through hole sidewalls, and minimizing resistive heating effects of electrodes on desired cells, are applicable here.

In one non-limiting embodiment, operations 410-450 can be repeated after objects (such as cells that are not of interest) are removed during operation 460. Repeating method 400, or certain operations in method 400, in this manner can result in a microfluidic chip having a large number of cells of interest captured in the filter membrane(s) of the microfluidic chip. Microfluidic chips with a maximum density of cells of interest can thus be obtained by repeating method 400, or certain operations of method 400, on the same microfluidic chip after objects not of interest are removed at each iteration of operation 460.

At optional operation 470, “Harvesting confirmed cells of interest,” captured objects that are identified as cells of interest are removed from the filter membrane for genetic and/or diagnostic analysis. For example, cells identified as cells of interest at operation 450 are next harvested at operation 470. In some embodiments, a micromanipulator may be used to harvest and/or pluck cells of interest from the through holes during operation 470. For example, a micromanipulator may include a needle configured to pluck cells captured in each individual through hole of the filter membrane. The needle tip and movement can be engineered so as not to puncture the filter membrane. The insertion and removal of the needle in each through hole may exert an outward force onto the sidewalls of a given through hole, thus the material, dimensions, and through hole density of the filter membrane can be selected to withstand this force such that the filter membrane does not break or the through hole is not deformed. In some cases, these advantageous mechanical properties of the filter membrane allow for a user to repeatedly use the same filter membrane to process a single sample, for example, by apply additional portions of the sample to the filter membrane after operation 450 and before harvesting all captured cells of interest at operation 470. In some aspects, harvesting of confirmed cells of interest in operation 470 is only performed after a significant number of through holes have retained confirmed cells of interest. Thus, the distinct, precisely-defined position of each through hole within the microfluidic chip enables the extraction and/or manipulation of captured cells that are of interest, in addition to manipulation and removal of objects (such as cells) that are not of interest.

### EXAMPLE 1

### Method of Fabricating a Microfluidic Chip Having Electrically-Controllable Through Holes

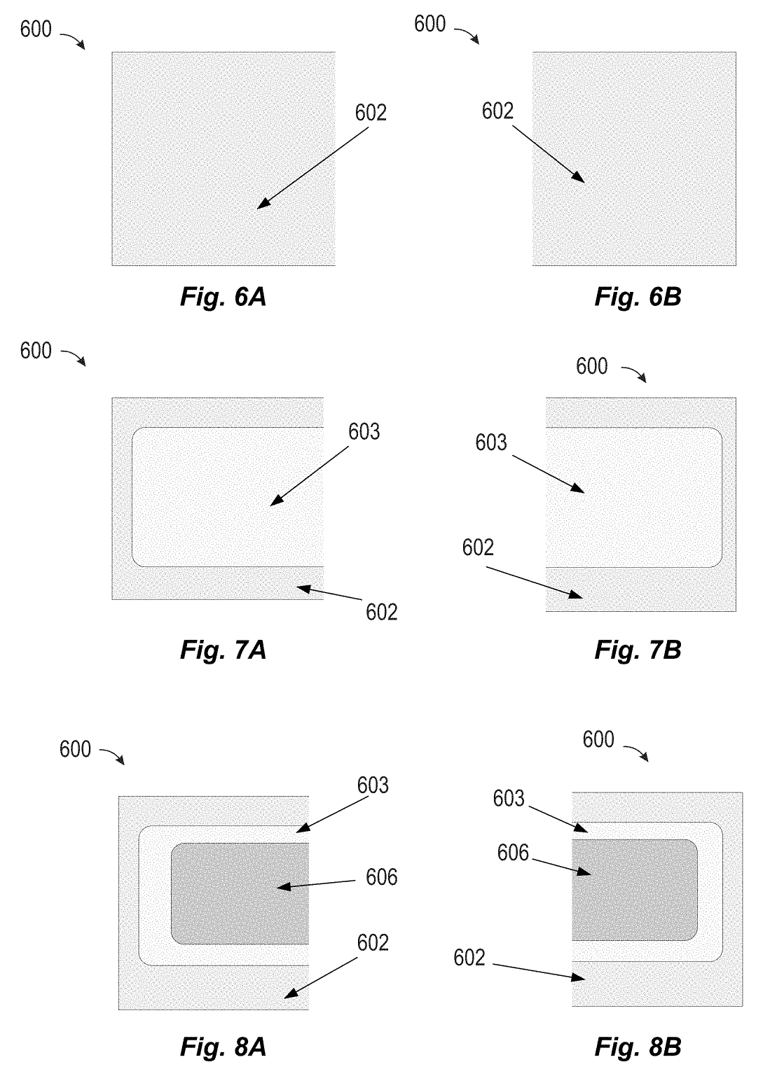

FIG. 5 is a flow diagram illustrating an example process 500 of fabricating microfluidic chips having electrically-controllable through holes as described herein. FIGS. 6A through 22B show example top view schematic illustrations of corresponding stages of such a fabrication process 500. FIGS. 6A, 7A, 8A, 9A, 10A, 11A, 12A, 13A, 14A, 15A, 16A, 17A, 18A, 19A, 20A, 21A, and 22A illustrate a microfluidic chip having generally ring-shaped electrodes and generally elliptical or oval-shaped through holes, whereas FIGS. 6B, 7B, 8B, 9B, 10B, 11B, 12B, 13B, 14B, 15B, 16B, 17B, 18B, 19B, 20B, 21B, and 22B illustrate a microfluidic chip having rhombus- or diamond-shaped electrodes and generally circular through holes. Other electrode and through hole shapes and combinations of shapes are possible. FIGS. 6A through 22B are schematic representations and are not drawn to scale. The features and aspects disclosed herein are intended to be illustrative and may be exaggerated in size to better illustrate the particular aspects of the embodiments described in each representative figure.

While the shapes and dimensions of the respective electrodes and through holes may differ from the non-limiting examples illustrated in FIGS. 6A through 22B, methods of fabricating embodiments of microfluidic chips described herein involve similar features. Therefore, the following describes a method of fabricating a microfluidic chip having electrically-controllable through holes with reference to the microfluidic chip having ring-shaped electrodes and elliptical through holes illustrated in FIGS. 6A, 7A, 8A, 9A, 10A, 11A, 12A, 13A, 14A, 15A, 16A, 17A, 18A, 19A, 20A, 21A, and 22A, but it will be understood that the same or a substantially similar process may be performed to develop a microfluidic chip having differently-shaped and differently dimensioned electrodes and through holes, such as those illustrated in FIGS. 6B, 7B, 8B, 9B, 10B, 11B, 12B, 13B, 14B, 15B, 16B, 17B, 18B, 19B, 20B, 21B, and 22B, or any other suitable configuration. Additionally, the steps illustrated in the FIG. 5 flow chart are preferably performed in the illustrated order; however, as will be understood by those skilled in the art, they may also be performed in other sequences and various substitutions and replacements may be made. In the discussion below, some of the possible substitutions and replacements will be discussed in further detail. Further, while omitted in the following description of process 500, appropriate cleaning steps can be performed periodically and as needed to prepare a given layer for a following processing step and/or to clean the layer based on the previously processed step.

As used herein, the term “wafer” will be used to describe an incomplete microfluidic chip, and the term “microfluidic chip” will be used to describe the completed integrated microfluidic chip. For example, FIGS. 6A through 22B each illustrate one embodiment of a stage of fabricating an integrated microfluidic chip, where wafer 600 refers to each stage in the process 500. For example, FIG. 3A illustrates one embodiment of the completed microfluidic chip 300a fabricated using the process 500, where each of FIGS. 6A, 7A, 8A, 9A, 10A, 11A, 12A, 13A, 14A, 15A, 16A, 17A, 18A, 19A, 20A, 21A, and 22A represents at least one stage of the fabrication process that concludes with the microfluidic chip 300a of FIG. 3A.

The process 500 begins at block 501, where a substrate 602 is provided as shown in FIG. 6A. The substrate 602 can be formed of any suitable material and have any suitable dimension to support the filter membrane formed later in the process 500. In some cases, the substrate 602 is a silicon wafer. The silicon wafer can be a commercially available, conventionally-sized wafer that is processed to obtain desired dimensions for the substrate 602. For example, a standard silicon wafer can be thinned down to have a thickness of approximately 400 microns. The thickness of the substrate 602 can be selected based on the needs of the particular application for which the microfluidic chip is intended. In some embodiments, the substrate 602 may be a solid or semi-solid substrate that may be planar in structure, i.e., substantially flat or having at least one flat surface. The planar substrate can be manufactured using solid substrates common in the fields of microfabrication, for example, silica-based substrates, such as glass, quartz, silicon or polysilicon, as well as other known substrates, for example, gallium arsenide, to ensure superior manufacturability and enhanced repeatedly of target dimensions. Microfabrication techniques, such as photolithographic techniques, wet chemical etching, micromachining (drilling, milling and the like), may be applied in the fabrication of portions of microfluidic chips, such as substrates, described herein. Alternatively, polymeric substrate materials may be used to fabricate the devices of the present disclosure, including, for example, polydimethylsiloxanes (PDMS), polymethylmethacrylate (PMMA), polyurethane, polyvinylchloride (PVC), polystyrene, polysulfone, polycarbonate, and the like. In the case of such polymeric materials, injection molding or embossing methods may be used to form the substrates. In such cases, original molds may be fabricated using any of the above described materials and methods. The assembled microfluidic chips may be treated with plasma to alter surface wet-ability where desired post assembly or preferably treated first and then assembled.

The process 500 continues to sub-process 502 where an electrode is formed. For example, block 502 represents the formation of an electrode that is substantially similar to the second electrode 350a described with reference to FIG. 3A. Formation of the electrodes in block 502 starts at block 503 with the deposition of a conductive layer 603 on the substrate 602 as shown in FIG. 7A. The conductive layer 603 can be formed of any suitable material having the sought after electrical properties. Exemplary conductive materials include gold, platinum, indium tin oxide, titanium nitride, etc. Deposition of the conductive material may be carried out using deposition techniques such as physical vapor deposition (PVD, e.g., sputtering), plasma-enhanced chemical vapor deposition (PECVD), thermal chemical vapor deposition (thermal CVD), E-Beam evaporation, or spin-coating a thin layer of the selected conductive material onto substrate 602.

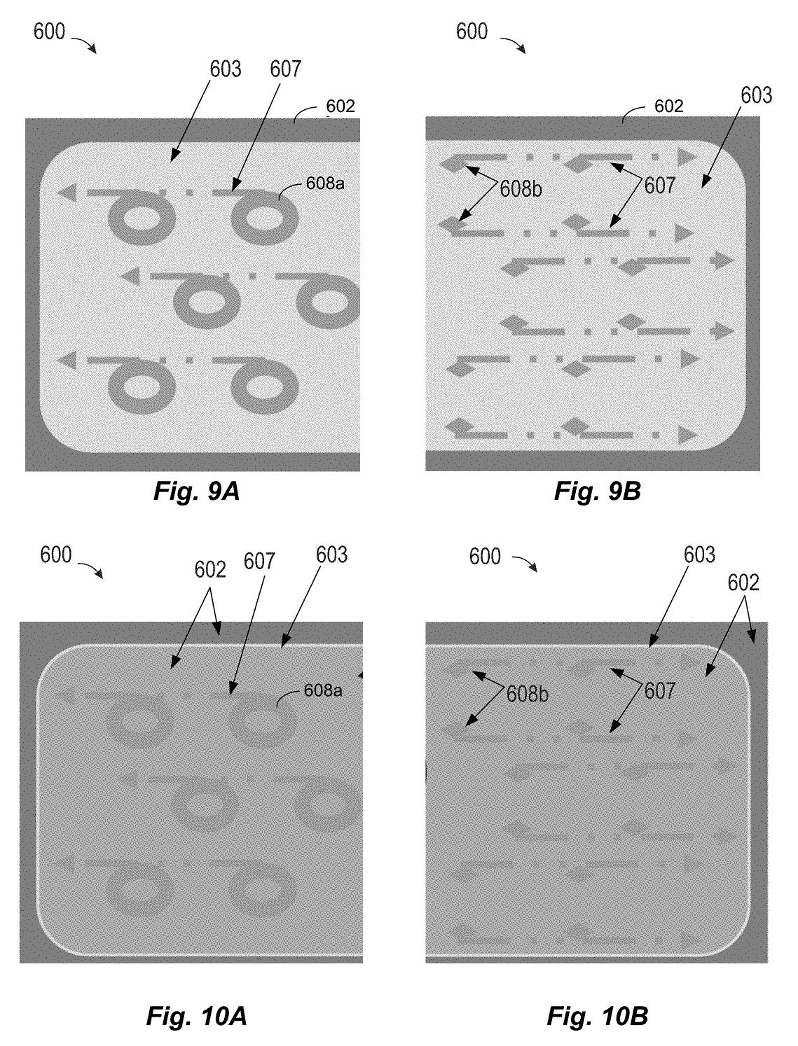

The sub-process 502 continues to block 504 where electrode patterns 608a are defined in a photoresist layer 606. The photoresist layer 606 may be deposited onto conductive layer 603, as shown in FIG. 8A, and patterned using conventional lithographic techniques (such as, but not limited to, hard mask, photoresist, exposure, development, and other suitable techniques). In one example embodiment using photoresist to define electrode patterns 608a in the conductive layer 603, a photoresist layer 606 is deposited through PVD, PECVD, thermal CVD, or spin-coating onto the conductive layer 603. Next, a mask (not shown) defining the electrode patterns 608a is applied over the photoresist layer 606. In one embodiment, the mask applied to the photoresist layer 606 is a negative of the ring-shaped electrodes such that the intended ring-shaped electrode patterns 608a are covered by the negative mask. In another embodiment, the mask is a negative of the rhombus-shaped electrodes patterns 608b (illustrated in FIG. 9B). Other configurations are possible. In yet another embodiment, electrode conducting line patterns 607 may also be patterned in the mask applied to the photoresist layer 606.

The mask applied to the photoresist 606 is configured to permit exposure of the areas of the photoresist layer intended to be removed, thereby leaving the material of the photoresist layer on the electrode patterns 608a (and the electrode conducting line patterns 607 if applicable). The wafer 600 is then exposed to light which causes a chemical change such that the exposed regions of the photoresist layer 606 are removed by a development step. The development step is performed by applying a developing solution to the surface of the microfluidic chip, the solution being configured to remove the exposed regions of the photoresist layer 606. The resulting wafer 600 is illustrated in FIGS. 9A and 9B where the photoresist layer 606 defines electrode patterns 608a on the conductive layer 603. In another non-limiting embodiment, patterning of the electrode structure may be performed using a lift-off technique.

The sub-process 502 continues to block 505 with the formation of electrodes 604a. In some embodiments, block 505 includes the formation of conducting lines 607 for electrical communication and selective control, as described above. Formation of the electrodes 604a is performed by etching away or removing portions of the conductive layer 603 that will not form part of a finished electrode 604a, where the portions are defined as the areas that remain uncovered by photoresist layer 606. Etching may be performed by numerous methods including, for example, chemical, physical, plasma, or wet etching process, where the etching removes areas of the conductive layer 603 unprotected by the remaining photoresist layer 606. In this step, the substrate 602 may act as an etch stop, whereby the etching process is stopped when it reaches the substrate 602. The resulting wafer 600 is illustrated in FIG. 10A and 10B, where electrodes 604a remain covered by the photoresist layer 606 (patterned into electrode patterns 608a) and the substrate layer 602 is exposed elsewhere.

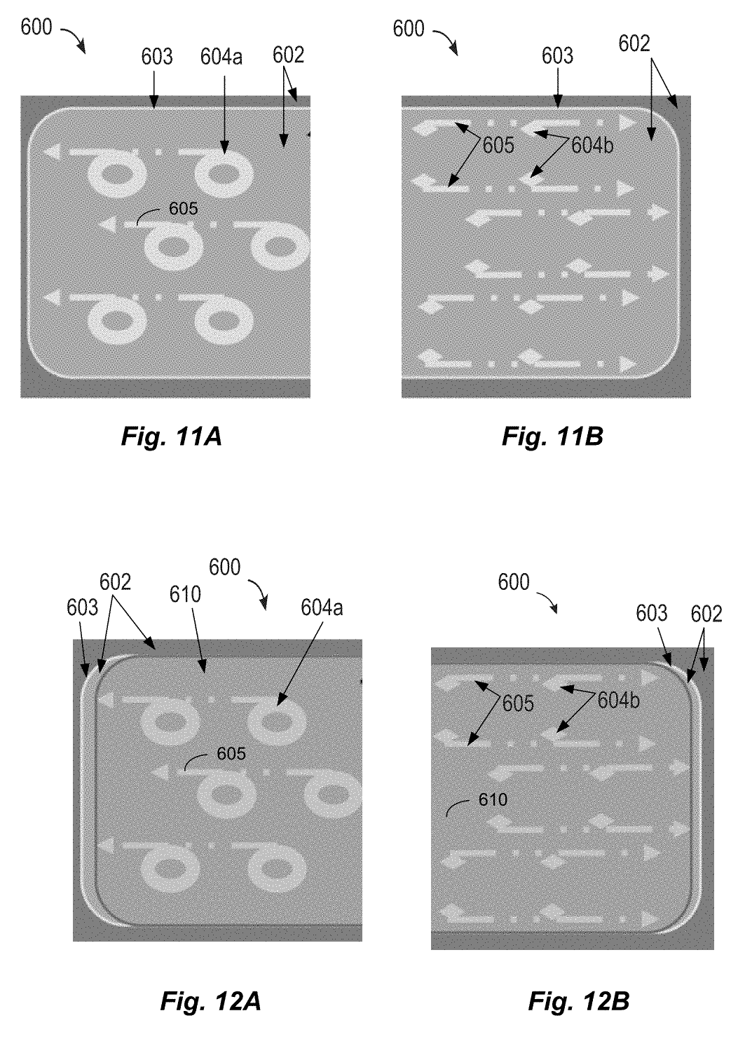

The sub-process 502 continues to block 506 with the removal of the photoresist layer 606 defining electrode patterns 608a in order to expose the electrodes 604a. The electrodes 604a comprise the remaining material left of the conductive layer 603 after etching in block 505. In some embodiments, removal of the remaining portions of the photoresist layer 606 in block 506 is performed by applying a liquid resist removal, where the photoresist layer 606 is altered such that it may be easily removed from the remaining conductive layer 603. In another embodiment, the photoresist layer 606 may be removed by ashing. Once the remaining portions of the photoresist layer 606 are removed, the electrode 604a formed from the selected conductive material is complete as illustrated in FIGS. 11A and 11B.

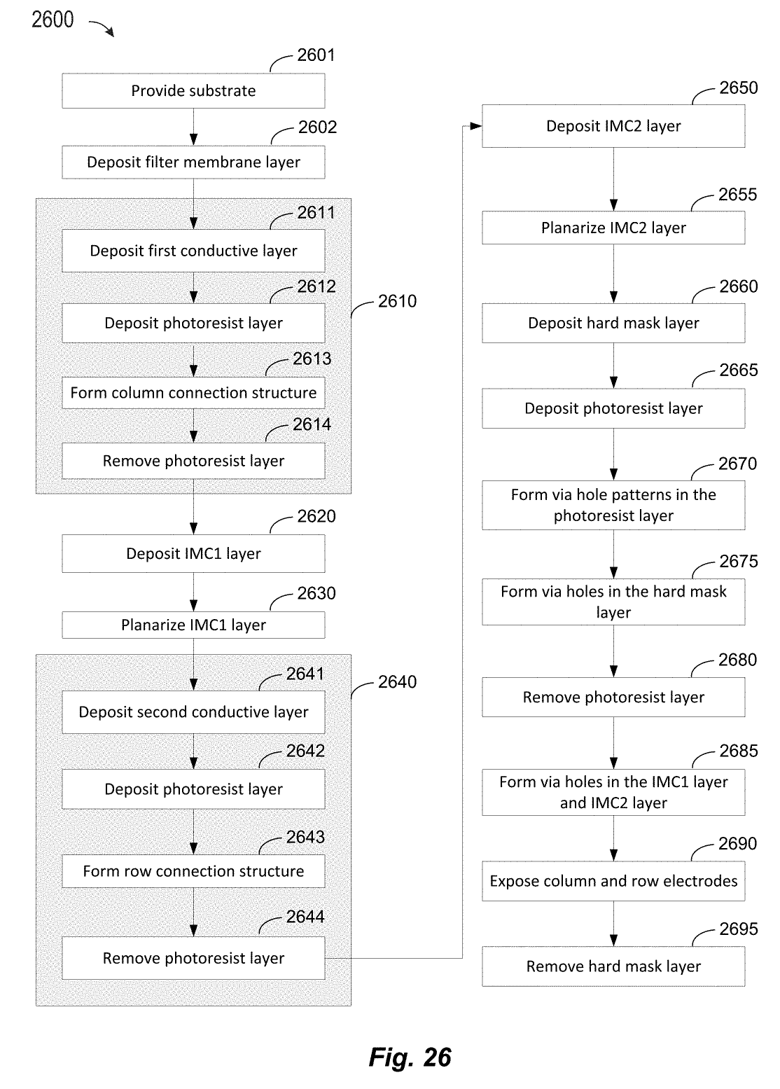

FIGS. 11A and 11B depict two exemplary electrode shapes (for example, ring-shaped electrode 604a and rhombus-shaped electrode 604b) disposed on substrate 602. Finished electrodes 604a and 604b include a suitable conductive material and are formed from conductive layer 602. The process of forming electrodes 604a and 604b may be representative of a process used to form second electrodes 350a and/or 350b as described with reference to FIGS. 3A and 3B. Further, electrical conducting lines 605, shown conceptually as lines and arrows leading from each electrode 604a and 604b, are also formed in the same processing steps from the same or similar conductive material in conductive layer 603. In some embodiments, further processing steps, described with reference to FIGS. 26 through 28B below, are included to form electrical connections (not shown) configured to apply an electrical current to electrical conducting lines 605 for controlling each individual electrode 604a and/or 604b, as described above with reference to block 460 of FIG. 4.

The process 500 next moves to sub-process 510 where a filter membrane having through holes is fabricated. In some embodiments, the filter membrane and through holes are substantially similar to those described herein with reference to FIGS. 1A through 4. For example, the filter membrane formed during sub-process 510 may be substantially similar to filter membrane 220 having through holes 205 extending between the first side 212 and second, opposing side 214 of the filter membrane 220. In another example, the filter membrane formed during sub-process 510 may be substantially similar to filter membrane 320a having through holes 305a extending between the first side 312 and second side 314 of the filter membrane 320a. It still another example, the filter membrane formed during sub-process 510 may be substantially similar to filter membrane 320b having through holes 305b extending between the first side 312 and second side 314 of the filter membrane 320b. Other configurations are possible.

The sub-process 510 begins at block 511 with the deposition of a filter membrane layer 610 as illustrated in FIGS. 12A and 12B. The filter membrane layer 610 can form all or a portion of a filter membrane that is substantially similar to a filter membrane 120 described with reference to FIGS. 1A, 1B, a filter membrane 220 described with reference to FIG. 2, a filter membrane 320a described with reference to FIG. 3A, a filter membrane 320b described with reference to FIG. 3B, or another filter membrane as described herein.

The filter membrane layer 610 may include any suitable dielectric material that provides suitable transparency, strength, and other physical properties for the intended cell capturing application, as described in more detail with reference to FIGS. 1A and 1B above. The filter membrane layer 610 should have minimal stress characteristics to withstand mechanical forces applied during sample fluid flow and/or physical manipulation of the through holes or its contents. The filter membrane layer 610 should also exhibit minimal fluorescence in the visible range (e.g., wavelengths of approximately 400 nm to approximately 700 nm) upon excitation from an external light source. The filter membrane layer 610 may be formed from silicon oxynitrides, such as but not limited to SiON or SiO₂. However, other materials are possible. The optical and fluorescence properties may be varied based on the specific systems, dyes, analysis, or operational requirements of the cell isolation and filtration processes. Further, the material used (for example, but not limited to, silicon or carbon/polymer based films) to form filter membrane layer 610 can be selected to meet the flow requirements and pressure fluctuations that are likely to be exerted by the system. Further still, the selected material can exhibit suitable wetting properties, such that it is hydrophilic or hydrophobic as required for the particular application of the microfluidic chip being manufactured. Specific requirements of these features according to at least one embodiment disclosed herein are described above with reference to FIG. 1.

Once a suitable material is selected for the filter membrane layer 610, deposition of the material may be carried out using deposition techniques such as physical vapor deposition (PVD), for example, sputtering, PECVD, thermal CVD, E-Beam evaporation, or spin-coating. The filter membrane layer 610 can be formed to have any suitable thickness for the particular application of the microfluidic chip. In some embodiments, the filter membrane layer 610 has a thickness of greater than or equal to 5 microns measured along a z-axis of the filter membrane layer 610. In one non-limiting example, the filter membrane layer 610 has a thickness of approximately 20 microns measured along a z-axis of the filter membrane layer 610. In one non-limiting example, the filter membrane layer 610 has a thickness of approximately. The precise thickness of the filter membrane layer 610 can be based on the required properties for the particular application of the microfluidic chip being manufactured.

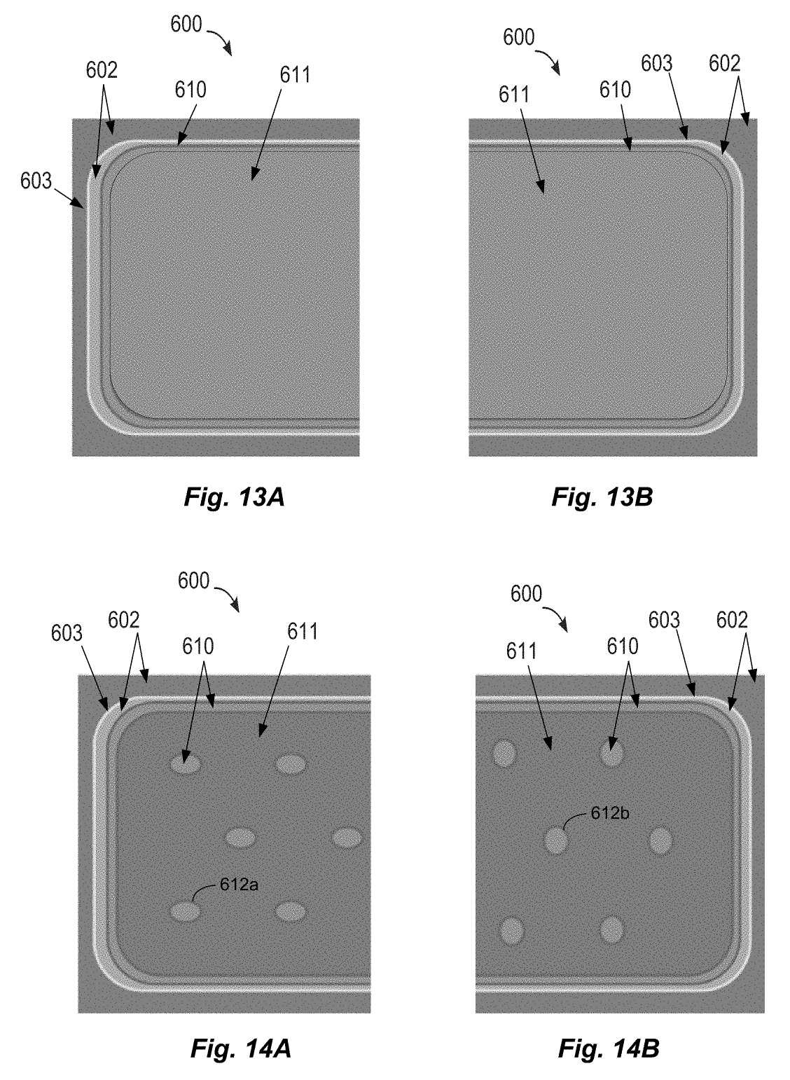

The sub-process 510 continues to block 512 where a photoresist layer 611 may be deposited onto filter membrane layer 610, as shown in FIGS. 13A and 13B, and patterned using conventional lithographic techniques (such as, but not limited to, hard mask, photoresist, exposure, development, and other suitable techniques). In one example embodiment using photoresist to define through holes 615a in the filter membrane layer 610, a photoresist layer 611 is deposited through PVD, PECVD, thermal CVD, or spin-coating onto filter membrane layer 610.

The sub-process 510 continues to block 513 where through holes are defined in photoresist layer 611 as illustrated in FIGS. 14A and 14B. A masking layer (not shown) may be deposited onto the photoresist layer 611 and patterned through lithographic techniques as described above in connection with block 504 to define through hole patterns 612a in the photoresist layer 611. Then, by exposing and developing the photoresist material left exposed by the mask in photoresist layer 611, through holes patterns 612a and 612b are defined and formed in the photoresist layer 611. The through hole patterns formed at block 513 and illustrated in FIGS. 14A and 14B only extend through the photoresist layer 611, and do not extend through the filter membrane layer 610. Thus, as shown in FIGS. 14A and 14B, the filter membrane layer 610 is viewable through the through hole patterns 612a and 612b formed in photoresist layer 611. The through hole patterns 612a and 612b define the eventual shape and size of the plurality of through holes to be formed in the filter membrane of the microfluidic chip. Therefore, although generally oval through hole patterns 612a are depicted in FIG. 14A and generally circular through hole patterns 612b are depicted in FIG. 14B, through hole patterns having other shapes are possible (for example, rectangular, square, etc.) as described above with reference to FIGS. 1A through 4.

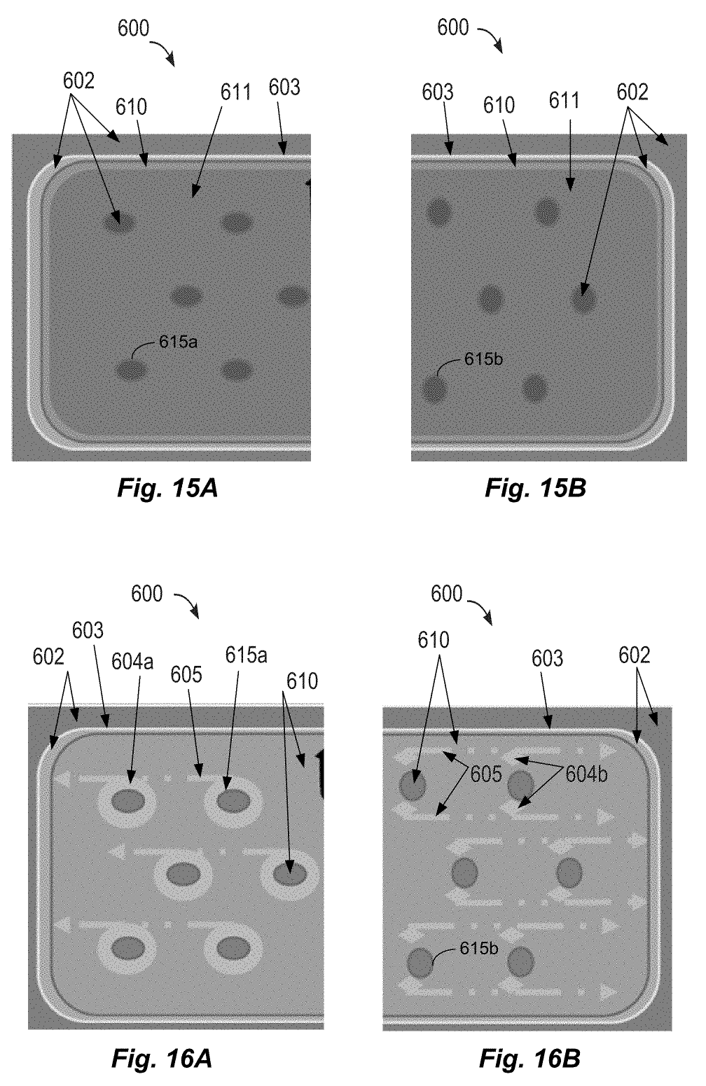

The sub-process 510 continues to block 514 where through holes 615a and 615b are formed in the filter membrane layer 610, as illustrated in FIGS. 15A and 15B. The through holes 615a and 615b are formed by etching away or removing portions of the filter membrane layer 610 that correspond to the through hole patterns 612a and 612b in the photoresist layer 611. Specifically, the portions of the filter membrane layer 610 that are removed in block 514 are the portions of the filter membrane layer 610 that were exposed when portions of photoresist layer 611 were removed during the developing stage of block 513. Etching may be accomplished by numerous methods, including but not limited to a chemical, physical, plasma, or wet etching process, where the etching removes areas of the filter membrane layer 610 that are not protected from etching by the portions of photoresist layer 611 remaining over the filter membrane layer 610. In block 514, the substrate 602 may act as an etch stop, whereby the etching process is stopped when it reaches the substrate 602. After the etching step at block 514, through holes 615a and 615b having desired dimensions and shapes for the particular filtering application are defined in the filter membrane layer 610. Thus, as shown in FIGS. 15A and 15B, the substrate 602 is viewable through the through holes 615a and 615b formed in the filter membrane layer 610. Additionally, the above-described process results in, with reference to the FIG. 15A embodiment, a plurality of through holes 615a arranged in a regular-repeating pattern, where each through hole 615a is located at a distinct, precisely-defined x, y location of the filter membrane layer 610. Similarly, with reference to the FIG. 15B embodiment, the above-described process results in a plurality of through holes 615b arranged in a regular-repeating pattern, where each through hole 615b is located at a distinct, precisely-defined x, y location of the filter membrane layer 610.

The sub-process 510 continues to block 515 with the removal of the photoresist layer 610. The photoresist layer 610 is removed in the substantially the same manner as performed in block 506. As shown in FIGS. 16A and 16B, once the photoresist layer 610 is removed, the filter material layer 610 is revealed including through holes 615a or 615b extending between the first surface of the filter membrane layer 610 to the second surface of the filter membrane layer 610. The combination of the filter membrane layer 610 and the through holes 615a or 615b formed in the filter membrane layer 610 represents one embodiment of a filter membrane, or a portion of a filter membrane, as described above with reference to FIGS. 1A through 4.

FIGS. 16A and 16B also depict electrodes 604a and 604b and electrical conducting lines 605 viewable through the optically transparent filter membrane layer 610. Through precise processing and alignment during the above-described processing steps, the through holes 615a and 615b can be precisely aligned with the electrodes 604a and 604b. In the example implementation illustrated in FIG. 16A, precise alignment results in a single through hole 615a positioned above the center of one ring-shaped electrode 604a. In the example implementation illustrated in FIG. 16B, precise alignment results in a single through hole 615b positioned between two rhombus-shaped electrodes 604b. This precise alignment permits individual electrode/through hole pairs to be precisely identified based on their distinct, precisely-defined locations within the filter membrane layer 610 (which forms a filter membrane or a portion of a filter membrane as described herein, including but not limited to filter membranes described above with reference to FIGS. 1A through 4). By precisely aligning and positioning the electrode relative to its corresponding through hole, the electrode/through hole pairs enable precise and independent control of a voltage bias applied to each through hole and any contents therein, as described above with reference to FIGS. 3A through 4.

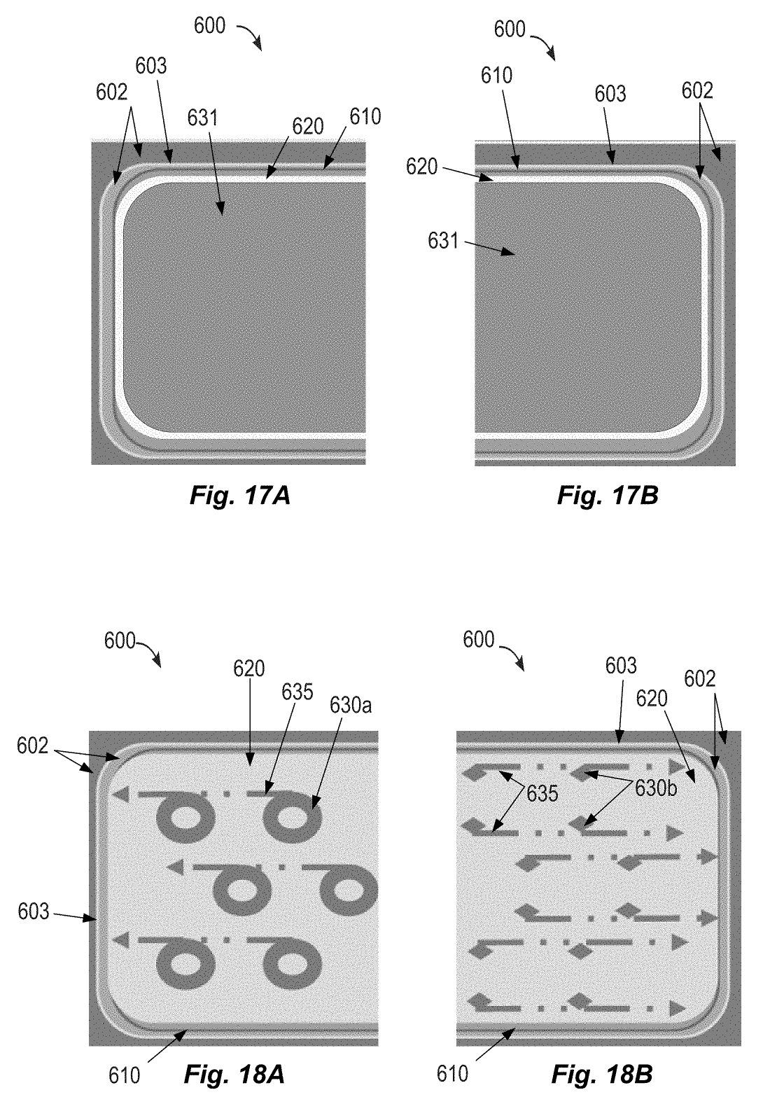

Once the through holes 615a are formed in the filter membrane layer 610, the process 500 continues to sub-process 520 where an electrode 640a is formed that may be functionally similar to first electrode 340a described with reference to FIG. 3A. The steps of sub-process 520 are substantially similar to steps of sub-process 502, however, a conductive material layer 620 is deposited on the filter membrane layer 610 instead of the substrate 602. Further the features, materials, and properties of the electrodes 640a or 640b may be substantially similar to those of electrodes 604a or 604b. Sub-process 520 starts at block 521 with the deposition of conductive material layer 620 on the surface of filter membrane layer 610, as illustrated in FIGS. 17A and 17B. The deposition of conductive material layer 620 is substantially similar to the deposition step of block 503. The sub-process 520 continues to block 522 where a photoresist layer 631 is deposited onto conductive material layer 620, as shown in FIG. 17A. The deposition of photoresist layer 631 may be performed in a substantially similar manner as described above with reference to photoresist layer 611.

The sub-process 520 continues to block 523 where a photoresist layer 631 is patterned using photolithographic techniques, as described above, to define electrode patterns 630a, electrode patterns 630b, and electrical conducting lines 635. FIG. 18A illustrates one example arrangement, where the conductive material layer 620 is deposited on the surface of the filter material layer 610 and includes electrode patterns 630a patterned in the photoresist layer 631 on top of the conductive material layer 620. FIG. 18B illustrates another example arrangement, where the conductive material layer 620 is deposited on the surface of the filter material layer 610 and includes electrode patterns 630b patterned in the photoresist layer 631 on top of the conductive material layer 620.

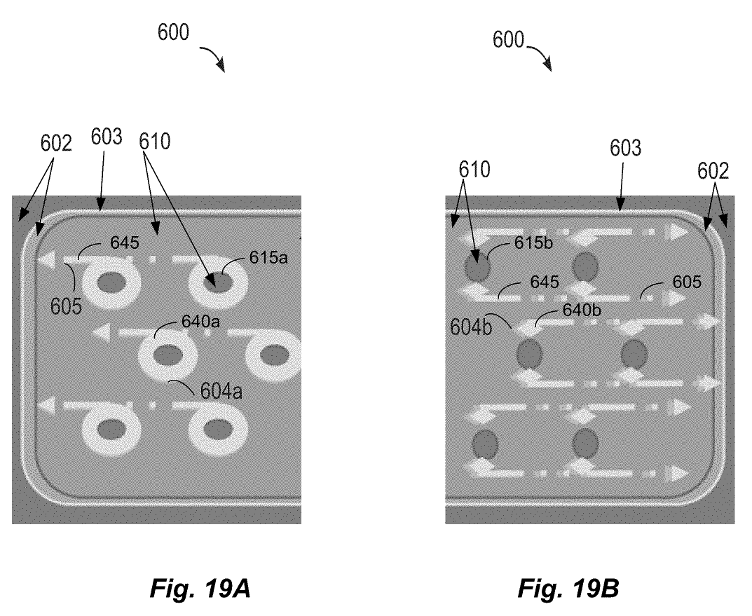

Sub-process 520 continues to block 524 with the formation of the electrodes 640a and 640b by an etching process that is substantially similar to the formation step of block 505. In some embodiments, lift-off techniques can be used where appropriate in place of etching techniques. In some embodiments, electrical conducting lines 645 are formed in the same processing step. The etching process may be configured to etch through the entirety of the exposed conductive material layer 620. In some embodiments, the filter membrane layer 610 may function as a etch stop. In some embodiments, further processing steps, described below with reference to FIGS. 26 through 28B, may be included to form electrical connections (not shown) configured to apply a voltage bias to the electrical conducting lines 645 for controlling the individual electrodes 640a, 640b independently, as described above in reference to block 460 of FIG. 4. Upon formation of the electrodes 640a, 640b, the sub-process 520 continues to block 525 with the removal of the photoresist layer 631. Removing the photoresist layer 631 may be performed in a substantially similar manner as in block 506, thereby revealing the underlying electrodes 640a, 640b as illustrated in FIGS. 19A and 19B.

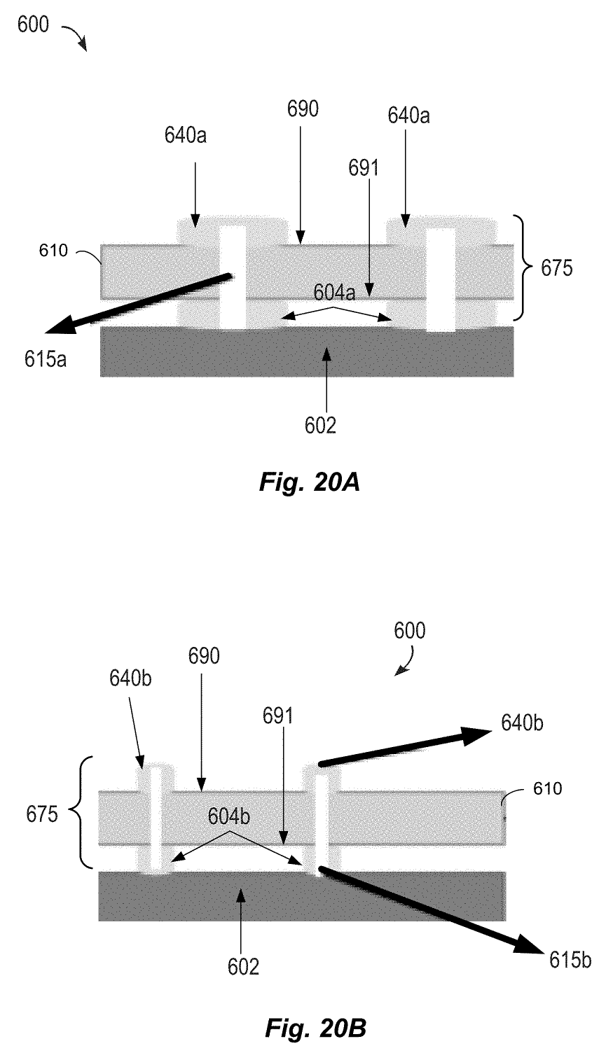

FIGS. 19A through 19B each illustrate a wafer 600 manufactured in accordance with the process 500 up to block 525. FIGS. 19A and 19B illustrate top down views of the processed wafer 600 up to block 525 of process 500. FIG. 20A illustrates a partial cross-sectional side view of the processed wafer 600 illustrated in FIG. 19A, and FIG. 20B illustrates a partial cross-sectional side view of the processed wafer 600 illustrated in FIG. 19B. The wafer 600 illustrated in FIGS. 19A and 20A are substantially the same as the wafer 600 illustrated in FIGS. 19B and 20B, however, the through holes 615a and 615b have different dimensions and orientations, and the electrodes 640a and 604a are of a different shape and configuration than electrodes 640b and 604b. For purposes of illustrating features that wafer 600 illustrated in FIGS. 19A and 20A and FIGS. 19B and 20B have in common, the following description will illustrate certain features of wafer 600 with reference to FIGS. 19A and 20A, but it will be understood that the described aspects of wafer 600 depicted in FIGS. 19A and 20A also apply to wafer 600 depicted in FIGS. 19B and 20B.

As illustrated, the wafer 600, at this point in the fabrication process 500, includes a substrate 602 having a filter membrane 675 disposed thereon. The filter membrane 675 includes a filter membrane layer 610 with through holes 615a formed by steps 511 through 514, where the through holes 615a extend between a first surface 690 of the filter membrane layer 610 and a second, opposing surface 691 of the filter material layer 610. The filter membrane 675 further includes two electrodes 604a and two electrodes 640a that are precisely aligned with two corresponding through holes 615a. The electrodes 604a are disposed between the substrate 602 and the second surface 691 of the filter membrane layer 610, while the electrodes 640a are disposed on the first surface 690 of the filter membrane layer 610. Further, electrodes 604a and 640a are electrically connected to electrical conducting lines 605 and 645 (where applicable), respectively, for applying an electrical current or voltage to the corresponding electrode. In this way, the voltage bias between two electrodes 604a and 640a associated with a single through hole 615a may be independently and individually controlled based on the distinct and precisely-defined location of each through hole in the filter membrane 675 in accordance with embodiments described herein.

Once the electrodes 640a and 640b are formed and the photoresist layer 631 is removed, the process 500 continues to sub-process 540 where vanes 670 are formed in substrate 602. Vanes 670 can be substantially similar to vanes 130, 230, and 330 described above with reference to FIGS. 1A through 3B. FIGS. 22A and 22B illustrate top down views of wafer 600 depicting a completed microfluidic chip, where the vanes 670 define a filter region 680 in the filter membrane 675 that is generally square with rounded edges. The filter region 680 defined in filter membrane 675 by vanes 670 may be substantially similar to the filter regions 125 and/or 225 as described above with reference to FIGS. 1A through 2. Other configurations are possible, for example vanes defining a square-shaped filter regions without rounded edges (for example, as illustrated in FIG. 2) or hexagonal-shaped filter regions (for example, as illustrated in FIGS. 1A and 1B). In one embodiment, the sub-process 540 is performed starting from the second surface 691 of the filter material layer 610. This may be accomplished based on a first surface 690 to second surface 691 alignment, where a protective layer 650 is disposed on the first surface 690 of filter material layer 610 to protect the features thereon as sub-process 540 is performed starting from the second surface 691 of the filter material layer 610.

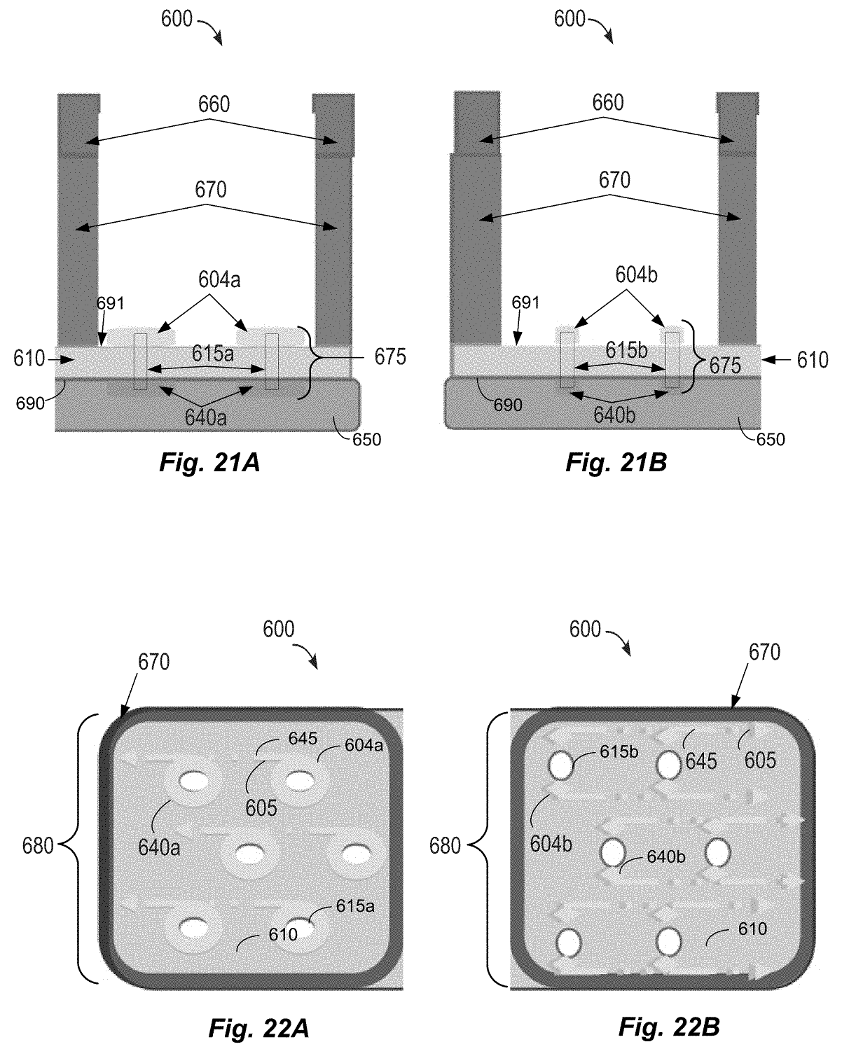

The sub-process 540 starts at block 541 with the deposition of a protective layer 650 as illustrated in FIGS. 21A and 21B. FIGS. 21A and 21B illustrate partial cross-sectional side views of wafer 600 having been flipped 180 degrees relative to the illustration of wafer 600 in FIGS. 20A and 20B. Thus, for example, the first surface 690 of filter membrane 675 in wafer 600 illustrated near the top of FIG. 20A is illustrated near the bottom of FIG. 21A. The protective layer 650 can be of any suitable material that functions to protect the electrodes 640a, 640b and the first surface 690 of the filter membrane 675. The protective layer 650 is also configured to be easily removed during a subsequent processing step without altering or affecting the electrodes 640a, 640b or the filter membrane 675.

Sub-process 540 continues to block 542 where a photoresist layer 660 is patterned using photolithographic techniques, as described above, to define the vanes 670. Sub-process 540 then moves to block 543 where vanes 670 are formed by etching the unpatterned substrate 602. Etching techniques to remove the unpatterned substrate 602 may be similar to those used in formation of electrodes 604a or 640a and/or in the formation of through holes 615a. In some embodiments, plasma dry etching is used. In other embodiments, wet etching chemistry is used with a hard mask to remove portions of the unpatterned substrate 602. For example, a SiN hard mask can be employed and potassium hydroxide (KOH) and isopropyl alcohol (IPA) mixtures may be used as a wet etchant. Upon formation of the vanes 670, the sub-process 540 continues to block 544 with the removal of the photoresist layer 660. Removing the photoresist layer 660 may be performed in a substantially similar manner as in block 506.

Following the removal of the photoresist layer 660, sub-process 540 continues to block 545 with the removal of the protective layer 650. The removal of protective layer 650 may be performed from the side of the wafer 600 closest to first surface 690 (corresponding to the side of the wafer 600 at the bottom of FIGS. 21A and 21B). The protective layer 650 may be formed of an etchable material (such as photoresist, polyimide, other polymer materials, in addition to a carrier wafer or another substrate bonded to the functional wafer with an intermediate and removable adhesive layer such as Crystal Bond wax) which may be removed by dry chemical etching, for example by exposing the protective layer 650 to a gaseous or vaporous etchant (such as vapors derived from oxygen plasma or downstream oxygen plasma) for a period of time that is effective to remove the desired amount of material. In the case of a bonded substrate, applied heat can release the bonded wafer, and typical oxygen plasma or surface cleans can be applied to clean the surface and remove any residue material. Other etching methods, e.g., wet etching and/or plasma etching, also may be used. Following removal of the protective layer 650, the side of the wafer 600 closest to first surface 690 (corresponding to the side of the wafer 600 at the bottom of FIGS. 21A and 21B) is appropriately cleaned to remove any residual material that may affect the fluid flow, optical, electrical, or mechanical characteristics of the completed microfluidic chip.

Once the vanes 670 are formed, the process 500 continues to block 550 with the dicing of wafer 600 into individual completed microfluidic chips. Wafer dicing can be accomplished using various techniques, including but not limited to laser dicing or mechanical sawing with a saw blade. In some embodiments, the dicing is accomplished using stealth dicing, which is a laser-based dicing technique where a defect is introduced into the wafer by scanning a laser-beam along the intended cutting line and then an underlying carrier (not shown) is expanded to induce fracture and separation of the wafer into individual microfluidic chips. Stealth dicing can advantageously permit the wafer surface to remain clean and minimize damage to the microfluidic chip due to vibrations that may be imposed during the dicing process. The individual microfluidic chips may then be packaged using suitable packaging techniques to protect the microfluidic chips.

Exemplary completed microfluidic chips having electrodes aligned with through holes in the filter membrane fabricated in accordance with process 500 are illustrated in FIGS. 3A and 3B. The features and functions of the microfluidic chips fabricated through process 500 are substantially similar to features and functions of microfluidic chips described throughout this disclosure, including but not limited to microfluidic chips described with reference to FIGS. 1A through 4.

### EXAMPLE 2

### Method of Fabricating a Microfluidic Chip Having Electrically-Controllable Through Holes

FIG. 23 illustrates a top down view of an exemplary microfluidic chip 2200 having filter membrane 2205 including a filter material layer 2210 with electrically-controllable through holes 2215. FIG. 23 is a schematic representation and is not drawn to scale. In this non-limiting example, the microfluidic chip 2200 is similar to the microfluidic chips 300a and/or 300b having a support layer and a filter layer with electrically-controllable through holes, however the through holes 2215 in this non-limiting embodiment have an elliptical or oval shape and the microfluidic chip 2200 includes a plurality of electrode/through hole pairs. Each electrode/through hole pair includes a through hole 2215, a first electrode 2204a, and a second electrode 2204b. In each electrode/through hole pair in this example implementation, electrodes 2204a and 2204b are disposed on the same side of the filter membrane 2205 (in contrast to the example implementations described with reference to FIGS. 3A through 22B, in which each electrode/through hole pair includes a first electrode disposed on one side of the filter membrane and a second electrode disposed on a second, opposing side of the filter membrane). The electrodes 2204a and 2204b are positioned relative to the through hole 2215 to allow an object (such as a cell) to enter the through hole 2215 through an aperture or opening 2215 separating the electrodes 2204a and 2204b. Accordingly, in this embodiment, the electrodes 2204a and 2204b are disposed on the same single surface of the filter membrane layer 2210. Without being bound to any particular theory, it is believed that positioning all of the electrodes associated with a single through hole on the same side of the through hole (disposed on the same single surface of the filter membrane through which the through hole passes) results in a microfluidic chip that is particularly suited to lyse, destroy, or fragment objects or cells captured in the through hole.

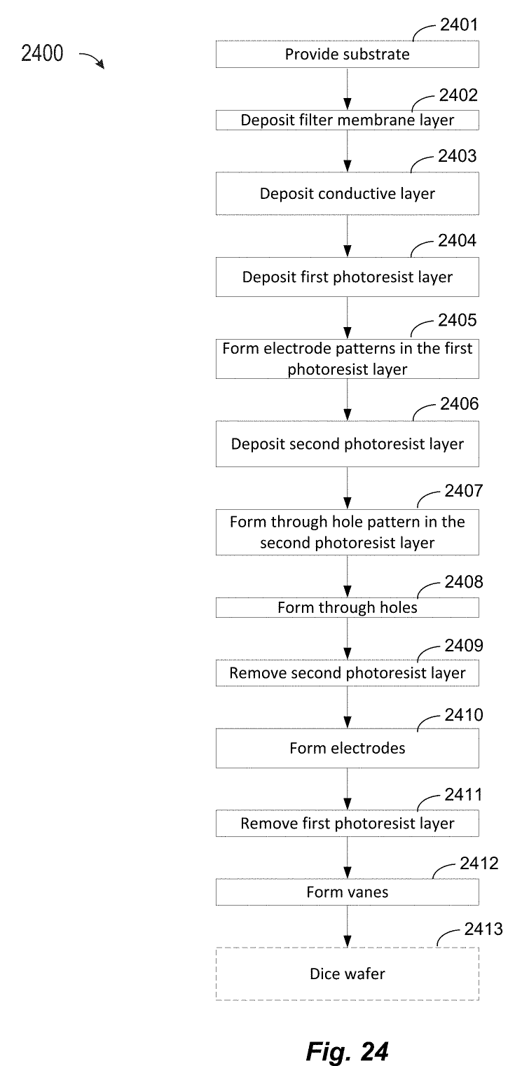

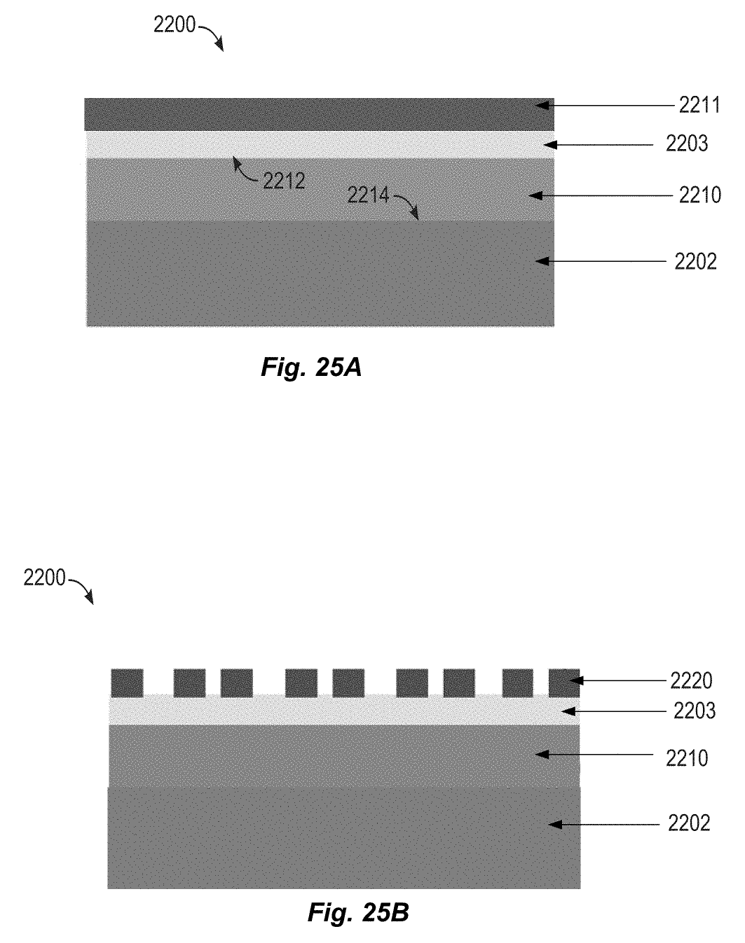

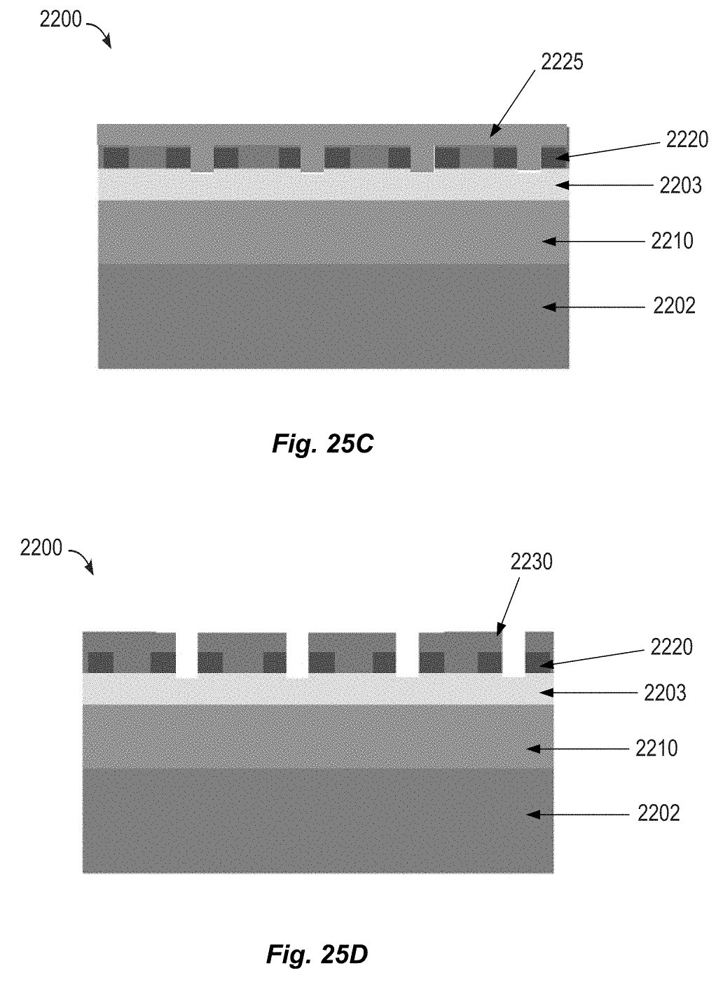

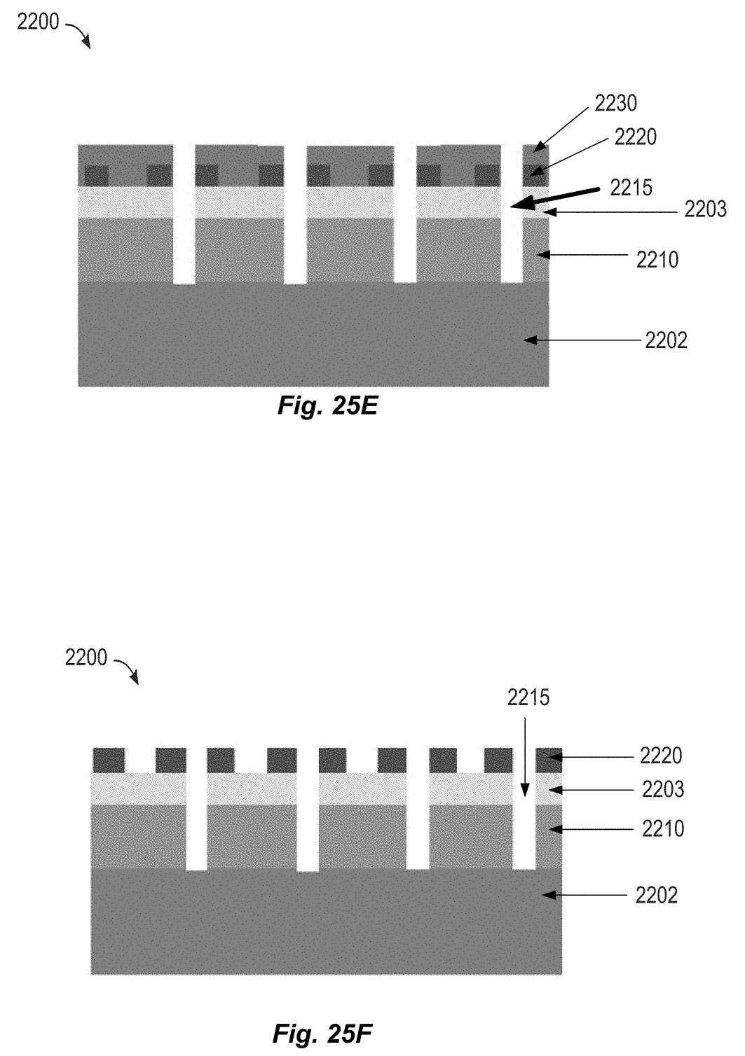

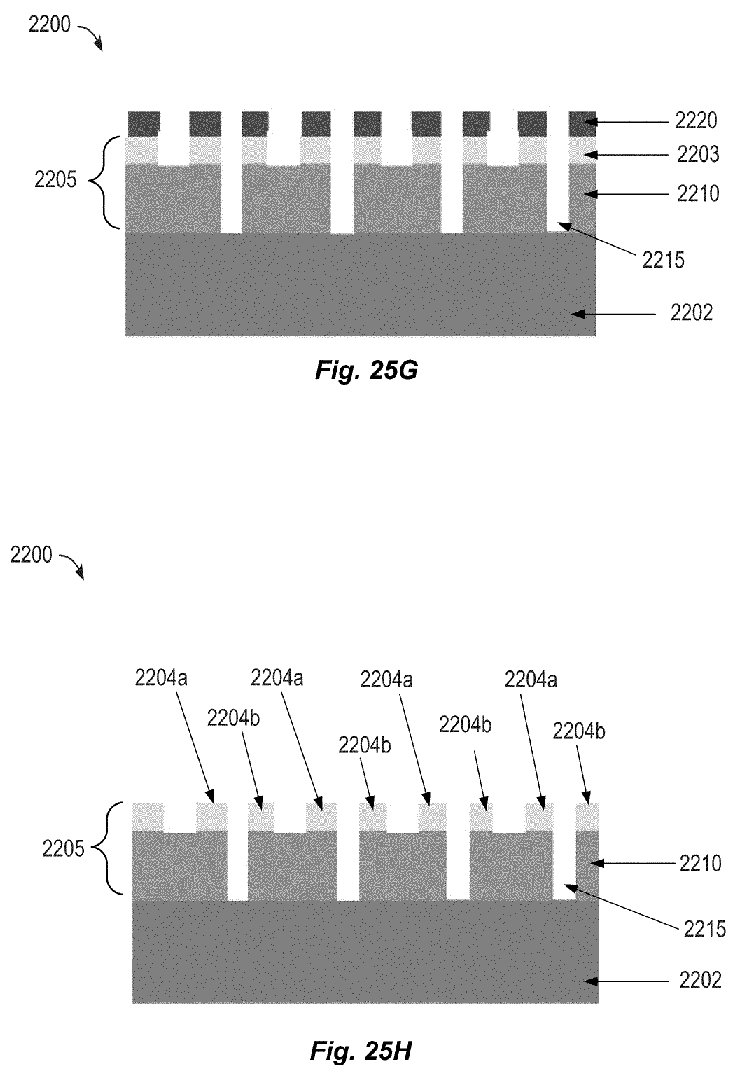

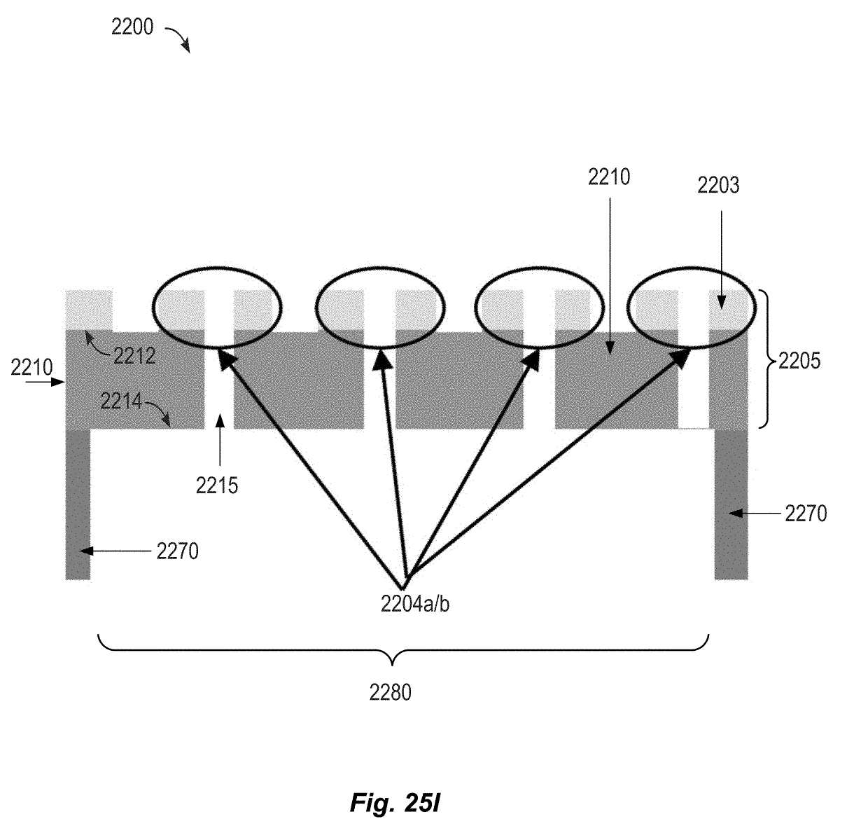

FIG. 24 is a flow diagram illustrating one example process 2400 of fabricating microfluidic chips having electrically-controllable through holes that are substantially similar to the electrode/through hole pairs described with reference to FIG. 3A through 22B. FIGS. 25A through 25I show example partial cross-sectional side view illustrations, taken along the line A-A of FIG. 23, of stages of the fabrication process 2400. FIGS. 25A through 25I are schematic representations and are not drawn to scale. The features and aspects disclosed herein are intended to be illustrative and may be exaggerated in size to better illustrate the particular aspects of the embodiments.

While the shapes and dimensions of the respective electrodes and through holes may differ from the non-limiting examples illustrated in FIGS. 3A through 22B, methods of fabricating embodiments of microfluidic chips described herein involve similar features. Therefore, the following describes a method of fabricating a microfluidic chip having electrically-controllable through holes with reference to the microfluidic chip having a plurality of electrode/through hole pairs, each electrode/through hole pair including two electrodes having a semi-circular shape and an oval-shaped through holes as described with reference to FIG. 23, but it will be understood that the same or a substantially similar process may be performed to develop a microfluidic chip having differently-shaped and differently dimensioned electrodes and through holes. Additionally, the steps illustrated in the FIG. 24 flow chart are preferably performed in the illustrated order; however, as will be understood by those skilled in the art, they may also be performed in other sequences and various substitutions and replacements may be made. In the discussion below, some of the possible substitutions and replacements will be discussed in further detail. Further, while omitted in the following description of process 2400, appropriate cleaning steps can be performed periodically and as needed to prepare a given layer for a following processing step and/or to clean the layer based on the previously processed step.

As used herein, the term “wafer” will be used to describe an incomplete microfluidic chip, and the term “microfluidic chip” will be used to describe the completed integrated microfluidic chip. For example, FIGS. 25A through 25I each illustrate one embodiment of a stage of fabricating an integrated microfluidic chip 220, where wafer 2500 refers to each stage in the process 2400. For example, FIG. 23 illustrates one embodiment of the completed microfluidic chip 2200 fabricated using the process 2400, where each of FIGS. 25A through 25I represents at least one stage of the fabrication process that concludes with the microfluidic chip 2200 of FIG. 23.

The process 2400 begins at block 2401, where a substrate 2202 is provided as shown in FIG. 25A. The substrate 2202 can be formed of any suitable material and have any suitable dimension to support the filter membrane formed later in the process 2400. Substrate 2202 may be substantially similar to substrate 602 as described with reference to FIGS. 5 through 22B. In some cases, the substrate 2202 is a silicon wafer. The thickness of the substrate 2202 can be selected based on the needs of the particular application for which the microfluidic chip is intended. The substrate 2202 may be manufactured using microfabrication techniques that are substantially similar to those described with reference to block 501 of FIG. 5.

The process 2400 continues to block 2402 with the deposition of a filter membrane layer 2210 on a surface of the substrate 220. The filter membrane layer 2210 can be substantially similar to filter membrane layer 610 described with reference to FIGS. 5 through 22B. For example, the filter membrane layer 2210 may include any suitable dielectric material that provides suitable transparency, strength, and other physical properties for the intended cell capturing application, as described in more detail with reference to FIGS. 1A and 1B above. Specific requirements of these properties according to at least one embodiment disclosed herein are described above with reference to FIGS. 1A and 1B.

Once a suitable material is selected for the filter membrane layer 2210, deposition of the material may be carried out in a substantially similar manner as described with reference to block 511 of FIG. 5. For example, filter membrane layer 2210 may be formed using deposition techniques such as PVD, PECVD, thermal CVD, E-Beam evaporation, or spin-coating. The filter membrane layer 2210 can be formed to have any suitable thickness for the particular application of the microfluidic chip. The filter membrane layer 2210 includes a first surface 2212 and a second, opposing surface 2214.

The process 2400 then moves to block 2403 with the deposition of a conductive material layer 2203 on the first surface 2212 of filter membrane layer 2210, as illustrated in FIG. 25A. The deposition of conductive material layer 2203 is substantially similar to the deposition of conductive layer 503 described with reference to block 503 of FIG. 5. The conductive material layer 2203 can be formed of any suitable material having the sought after electrical properties. Exemplary conductive materials include gold, platinum, indium tin oxide, titanium nitride, etc. Deposition of the conductive material may be carried out using deposition techniques as described above.

The process 2400 then moves to block 2404 where a first photoresist layer 2211 is deposited onto conductive material layer 2203, as shown in FIG. 25A. The deposition of first photoresist layer 2211 may be performed in a substantially similar manner as described above with reference to photoresist layer 606 of FIGS. 8A and 8B. In one example embodiment, the first photoresist layer 2211 is configured to define electrode patterns 2204a and 2204b in the conductive material layer 2203. After depositing first photoresist layer 2211, a mask (not shown) defining electrode patterns 2220 is applied over the first photoresist layer 2211. In one embodiment, the mask applied to the photoresist layer 2211 is a negative of the semi-circular shaped electrodes 2204a, 2204b such that portions of electrode patterns 2220 that will correspond to portions of the semi-circular shaped electrodes 2204a, 2204b are covered by the negative mask. Other configurations are possible.

The process 2400 then moves to block 2405 where the electrode patterns 2220 are formed in the first photoresist layer 2211. The mask applied to the first photoresist 2211 is configured to permit exposure of the areas of the first photoresist layer 2211 intended to be removed, thereby leaving the material of the first photoresist layer 2211 forming the electrode patterns 2220. The wafer 2500 is then exposed to light which causes a chemical change such that the exposed regions of the first photoresist layer 2211 are removed by a development step. The development step is performed by applying a developing solution to the surface of the microfluidic chip, the solution being configured to remove the exposed regions of the first photoresist layer 2211. The resulting wafer 2500 is illustrated in FIG. 25B where the remaining first photoresist layer 2211 defines electrode patterns 2220. In another non-limiting embodiment, patterning of the electrode structure may be performed using a lift-off technique.

The process 2400 continues to block 2406 where a second photoresist layer 2225 is deposited onto the electrode patterns 2220 and conductive material layer 2203, as shown in FIG. 25C. The deposition of second photoresist layer 2225 may be performed in a substantially similar manner as described above with reference to first photoresist layer 2211. The second photoresist layer 2225, however, is configured to define through holes 2215 in the filter membrane layer 2210 by defining a through hole pattern 2230 in the photoresist layer 2225. After depositing second photoresist layer 2225, a mask (not shown) defining the through hole patterns 2230 is applied over the second photoresist layer 2225. In one embodiment, the mask applied to the second photoresist layer 2225 is a negative of the through holes such that portions of through hole patterns 2230 that will correspond to through holes 2215 are covered by the negative mask. Other configurations are possible.

The process 2400 then moves to block 2407 where the through hole patterns 2230 are formed in the second photoresist layer 2225. The process of forming the through hole patterns 2230 in the second photoresist is substantially similar to the formation of the electrode patterns in the first photoresist 2211 (for example, exposing the masked photoresist layer and performing a development step to remove the mask). The resulting wafer 2500 is illustrated in FIG. 25D where the remaining second photoresist layer 2225 defines through hole patterns 2230.

The process 2400 then moves to block 2408 with the formation of the through holes 2215. Formation of the through holes 2215 is accomplished by etching away or removing portions of the wafer defined as areas that remain uncovered by the second photoresist layer 2225. In this regard, portions of the conductive layer 2203 that will not form part of finished electrodes 2204a or 2204b are etched away or removed as well. Then, portions of the filter membrane layer 2210 are etched away that correspond to the through hole patterns 2230. Specifically, the portions of the filter membrane layer 2210 that are removed in block 2408 are the portions of the filter membrane layer 2210 that were exposed when portions of photoresist layer 2225 were removed during the developing stage of block 2407, as shown in FIG. 25E. In block 2408, the substrate 2202 may act as an etch stop, whereby the etching process is stopped when it reaches the substrate 2202. In some embodiments, etching into substrate 2202 (e.g., over-etching) is permitted because portions of substrate 2202 aligned with the through holes 2215 are sacrificial and may be removed in subsequent processing steps. After the etching step at block 2408, through holes 2215 having desired dimensions and shapes for the particular filtering application are defined in the filter membrane layer 2210. Thus, as shown in FIG. 25E, the substrate 2202 is viewable through the through holes 2215 formed in the filter membrane layer 2210. Additionally, the above-described process results in a plurality of through holes 2215 arranged in a regularly-repeating pattern, where each through hole 2215 is located at a distinct, precisely-defined x, y location of in the filter membrane layer 2210. The resulting wafer 2200 is illustrated in FIG. 25E, where through holes 2215 are formed in the filter membrane 2210 and electrode patterns 2220 remain covered by the through hole patterns 2230 formed in the second photoresist layer 2225.

The process 2400 continues to block 2409 with the removal of the second photoresist layer 2225 in order to expose the electrode patterns 2220, as shown in FIG. 25F. In some embodiments, removal of the remaining portions of the second photoresist layer 2225 in block 2409 is performed by applying a liquid resist removal, where the second photoresist layer 2225 is altered such that it may be easily removed while the remaining first photoresist layer 2211 defining the electrode patterns 2220 remains unaltered. In another embodiment, the second photoresist layer 2225 may be removed by ashing. Once the remaining portions of the second photoresist layer 2225 are removed, the first photoresist layer 2211 defining electrode patterns 2220 remains unaltered for subsequent processing and formation of the electrodes 2204a and 2204b.

The process 2400 continues to block 2410 with the formation of electrodes 2204a and 2204b. Formation of the electrodes 2204a and 2204b is performed by etching away or removing portions of the conductive material layer 2203 that will not form part of finished electrodes 2204a and 2204b, where the portions are defined as the areas that remain uncovered by first photoresist layer 2211. Etching may be performed in a substantially similar manner to etching performed in block 2408, where the etching removes areas of the conductive material layer 2203 unprotected by the remaining first photoresist layer 2211. In this step, the filter membrane layer 2210 may act as an etch stop, whereby the etching process is stopped when it reaches the filter membrane layer 2210. The resulting wafer 2500 is illustrated in FIG. 25G, where electrodes 2204a and 2204b remain covered by the first photoresist layer 2211 (patterned into electrode patterns 2220). It is noted, that the etching step may etch into portions of the substrate 2202 exposed in the through holes 2215, having been processed in block 2409, however, these portions of the substrate 2202 are sacrificial and over-etching of the substrate 2202 is permissible. At this stage in the process 2400, filter membrane 2205 comprises a plurality of through holes 2215, each through hole 2215 associated with a pair of electrodes, electrodes 2204a and 2204b, disposed on the same single side of the through hole 2215 and configured to electrically control the through hole 2215 upon application of an voltage bias to the electrodes 2204a, 2204b.

The process 2400 continues to block 2411 with the removal of the first photoresist layer 2211 defining electrode patterns 2220 in order to expose the electrodes 2204a and 2204b. The electrodes 2204a and 2204b comprise the remaining material left of the conductive material layer 2203 after etching in block 2410. In some embodiments, removal of the remaining portions of the first photoresist layer 2211 in block 2411 is performed in a substantially similar manner to removal of the second photoresist layer 2225 in block 2409, where the first photoresist layer 2211 is altered such that it may be easily removed from the remaining conductive material layer 2203. Once the remaining portions of the first photoresist layer 2211 are removed, the electrode 2204a and 2204b formed from the selected conductive material is complete as illustrated in FIG. 25H.

FIGS. 25H and 23 depict an exemplary electrode shape (for example, semi-circular shaped electrodes 2204a and 2204b) disposed on the same single side of the filter membrane layer 2210 to form filter membrane 2205. Finished electrodes 2204a and 2204b include a suitable conductive material and are formed from conductive material layer 2203. In some embodiments, further processing steps, described with reference to FIGS. 26 through 28B below, are included to form electrical connections (not shown) configured to apply an electrical current for controlling each individual electrode 2204a and 2204b, as described above with reference to block 460 of FIG. 4.

Through precise processing and alignment during the above-described processing steps, each through hole 2215 of a plurality of through holes 2215 is precisely aligned with a pair of electrodes, electrodes 2204a and 2204b. In the example implementation illustrated in FIG. 25A through 25I, precise alignment results in a single through hole 2215 associated with a pair of side-by-side electrodes 2204a and 2204b. This precise alignment permits individual electrode/through hole pairs to be precisely identified based on their distinct, precisely-defined locations within the filter membrane layer 2210 (which forms a filter membrane or a portion of a filter membrane as described herein, including but not limited to filter membranes described above with reference to FIGS. 1A through 22B). By precisely aligning and positioning each electrode relative to its corresponding through hole, the electrode/through hole pairs enable precise and independent control of a voltage bias applied to each through hole and any contents therein, as described above with reference to FIGS. 3A through 4.

Once the electrodes 2204a or 2204b are formed and the first photoresist layer 2211 is removed, the process 2400 continues to block 2412 where vanes 2270 are fabricated. Vanes 2270 can be substantially similar to vanes 130, 230, 330, and 670 described above with reference to FIGS. 1 through 3B and 21A through 22B. The filter region 2280 defined in filter membrane 2205 by vanes 2270 may be substantially similar to the filter regions 125 and/or 225 and/or 680 as described above with reference to FIGS. 1A, 1B, 2, 22A, and 22B. Other configurations are possible, for example vanes defining square-shaped filter regions without rounded edges (for example, as illustrated in FIG. 2) or hexagonal-shaped filter regions (for example, as illustrated in FIGS. 1A and 1B).

The process at block 2412 for fabricating vanes 2270 is substantially similar to the sub-process 540. In one embodiment, block 2412 is performed starting from the second surface 2214 of the filter membrane layer 2210. This may be accomplished based on a first surface 2212 to second surface 2214 alignment, where a protective layer (not shown) is disposed on the first surface 2212 of filter membrane layer 2210 to protect the features thereon. As in sub-process 540 of FIG. 5, block 2412 starts with the deposition of a protective layer of any suitable material that functions to protect the electrodes 2204a and 2204b and the first surface 2212 of the filter membrane layer 2210. A photoresist layer (not shown) is deposited and patterned, as described in sub-process 540 of FIG. 5, to define the vanes 2270. The vanes 2270 are then formed by etching the unpatterned substrate 2202. The photoresist is then removed in a substantially similar manner as in block 544. The protective layer is also removed in a manner substantially similar to block 545 of FIG. 5. Following removal of the protective layer, the side of wafer 2500 corresponding to the first side 2212 of filter membrane layer 2210 is appropriately cleaned to remove any residual material that may affect the fluid flow, optical, electrical, or mechanical characteristics of the completed microfluidic chip.

Once the vanes 2270 are formed, the process 2400 optionally continues to block 2413 with the dicing of wafer 2500 into individual completed microfluidic chips. The individual microfluidic chips may then be packaged using suitable packaging techniques to protect the microfluidic chips.

An exemplary completed microfluidic chip having electrodes aligned with through holes in the filter membrane fabricated in accordance with process 2400 is illustrated in FIG. 23. The features and functions of the microfluidic chips fabricated through process 2400 are substantially similar to features and functions of microfluidic chips described throughout this disclosure, including but not limited to microfluidic chips described with reference to FIGS. 1A through 4 and 5 through 22B.

### EXAMPLE 3

### Method of Fabricating a Microfluidic Chip Having Electrically-Controllable Through Holes

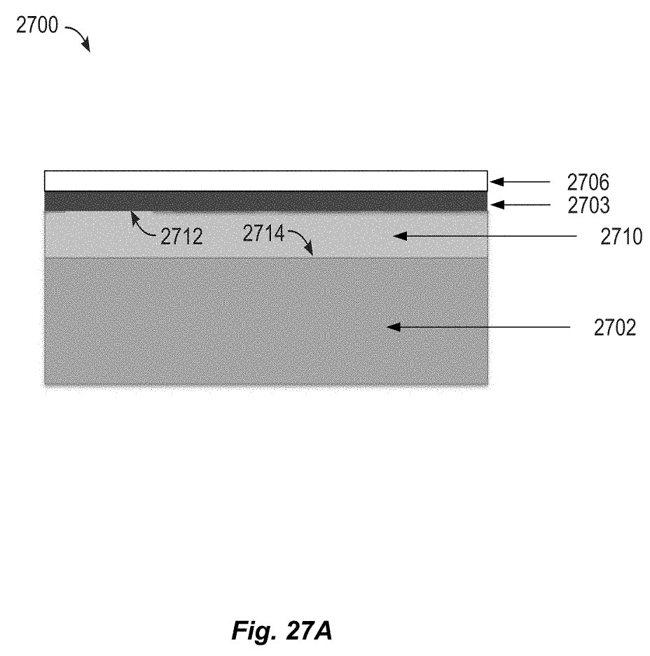

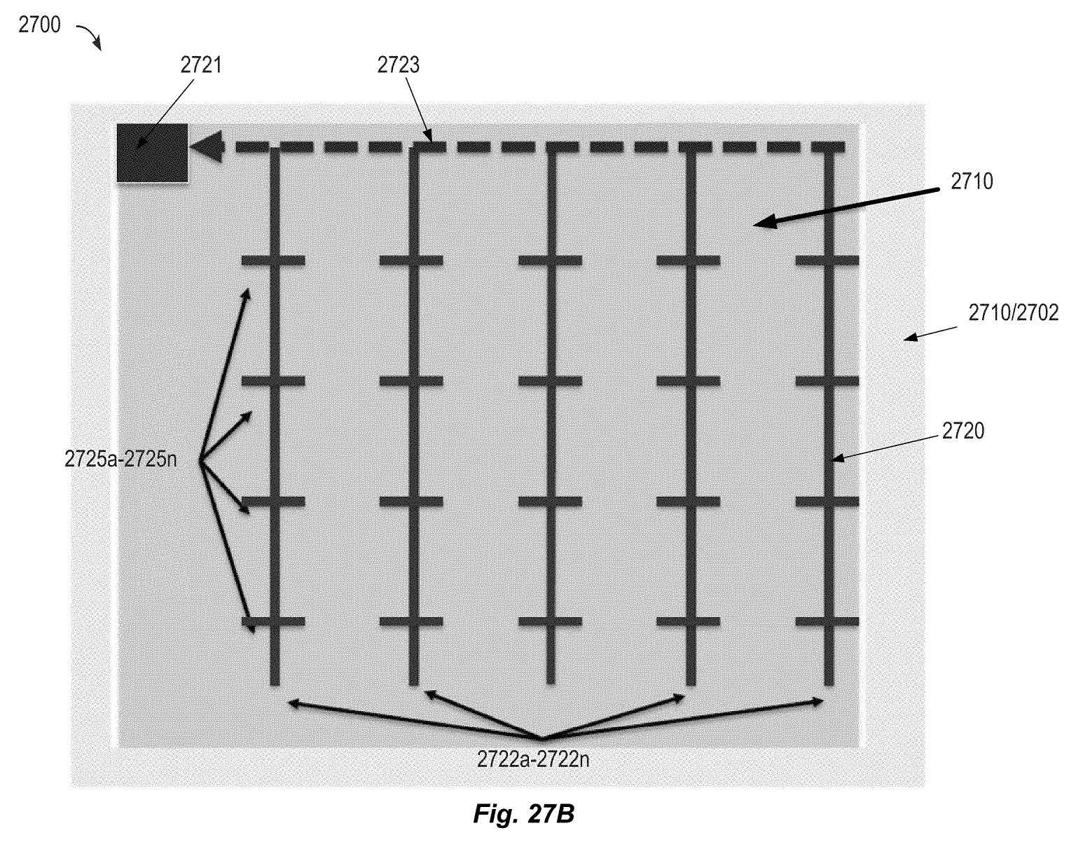

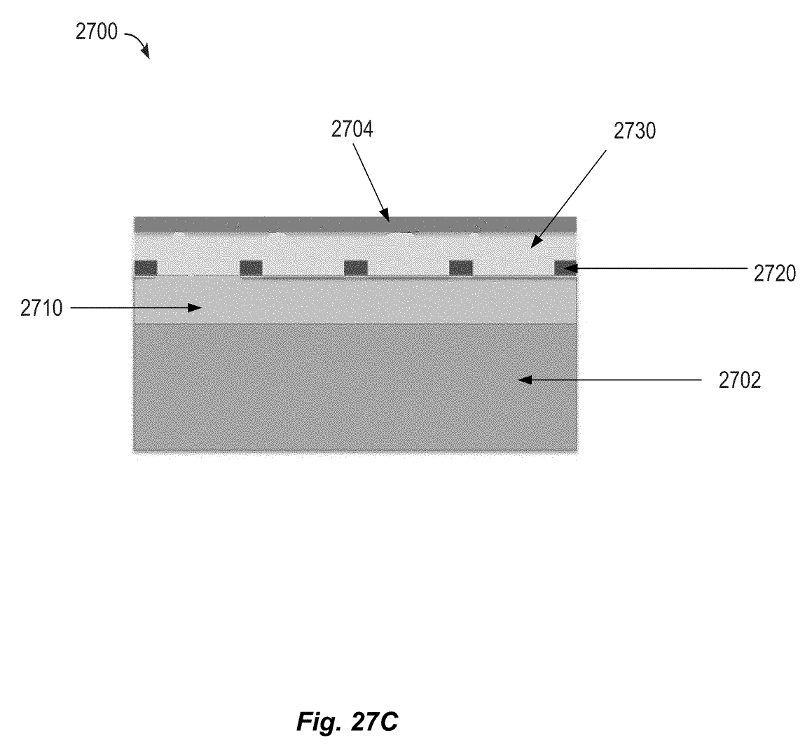

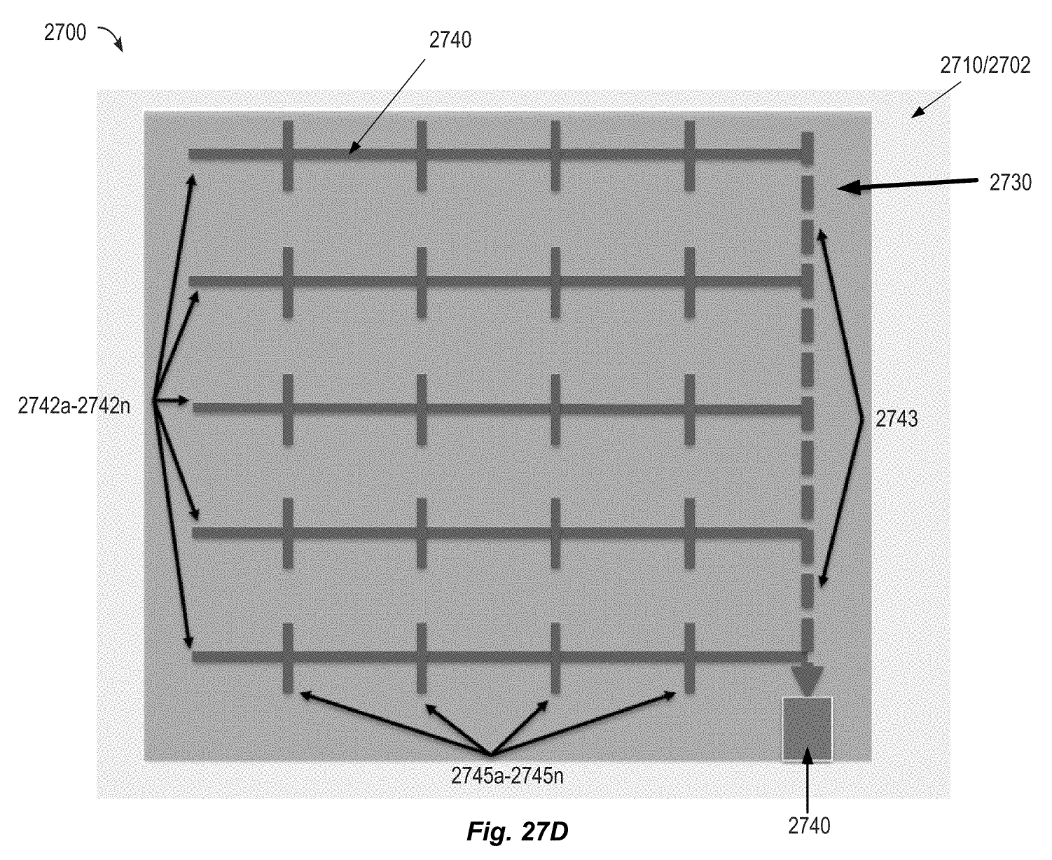

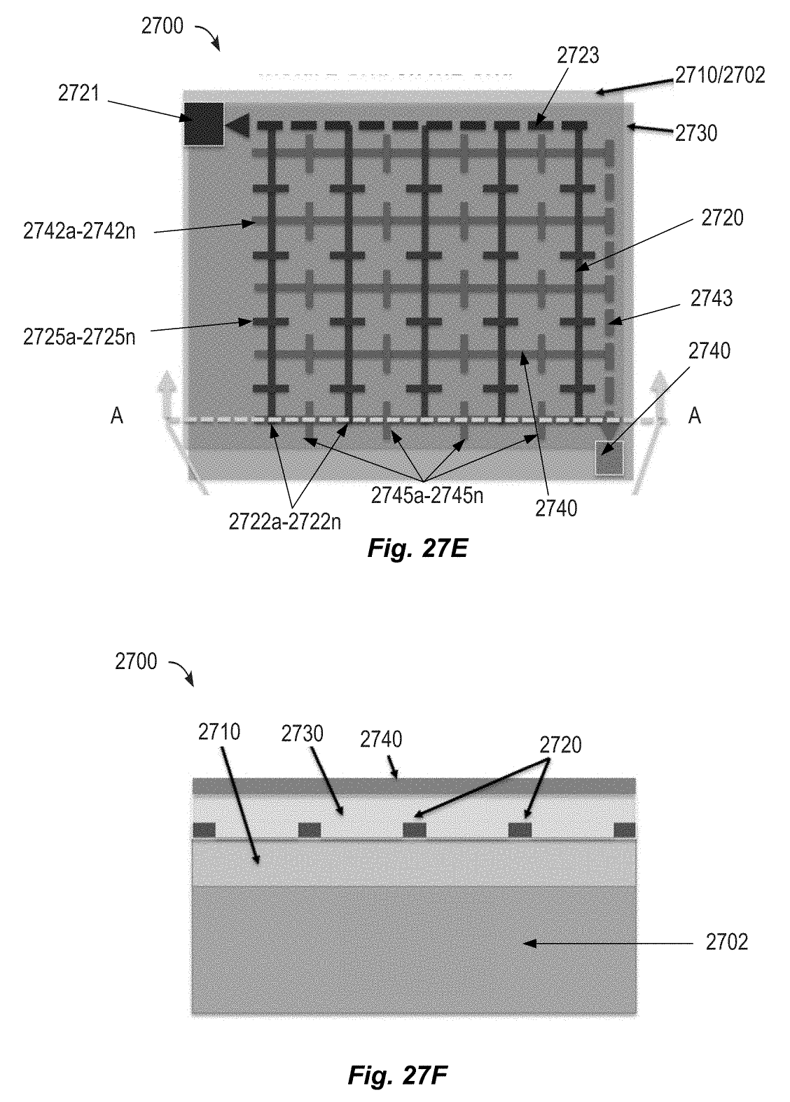

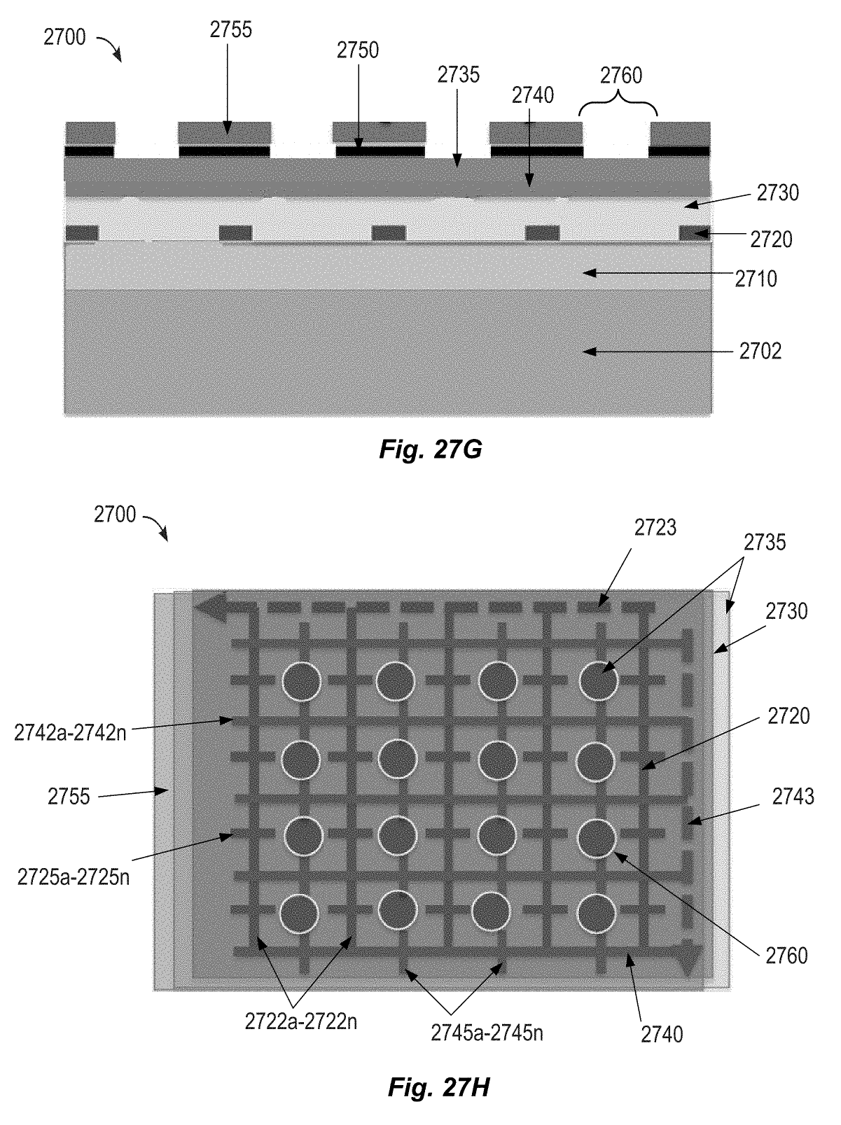

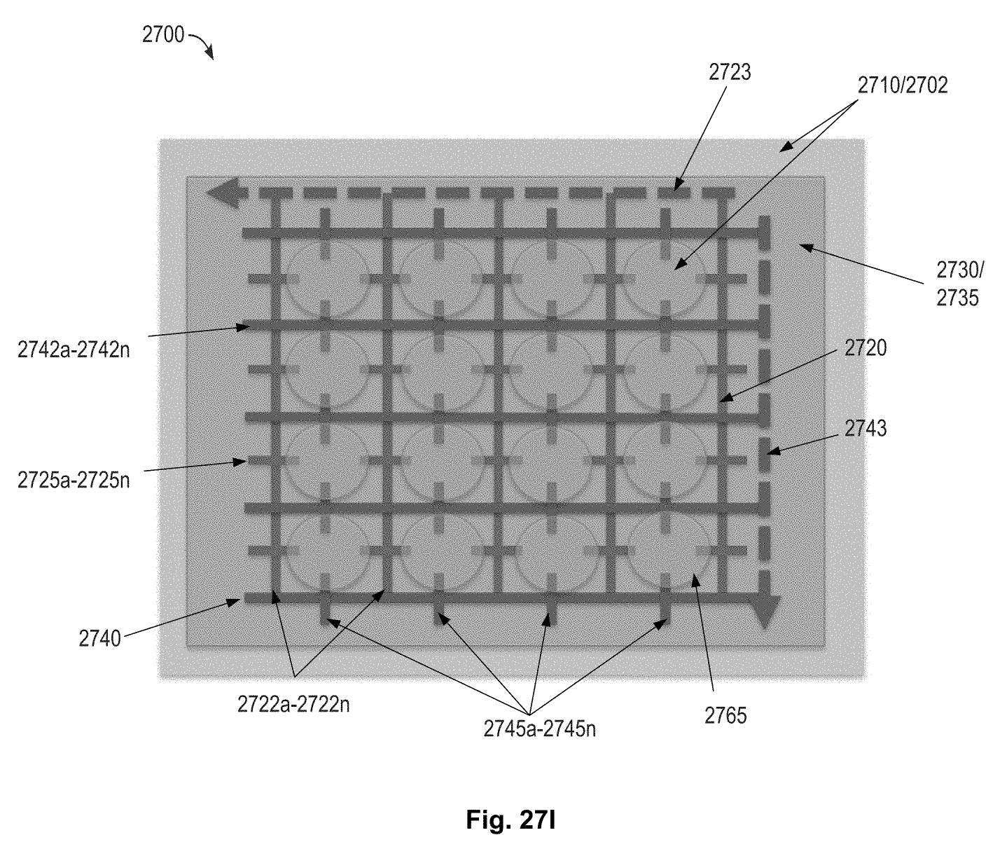

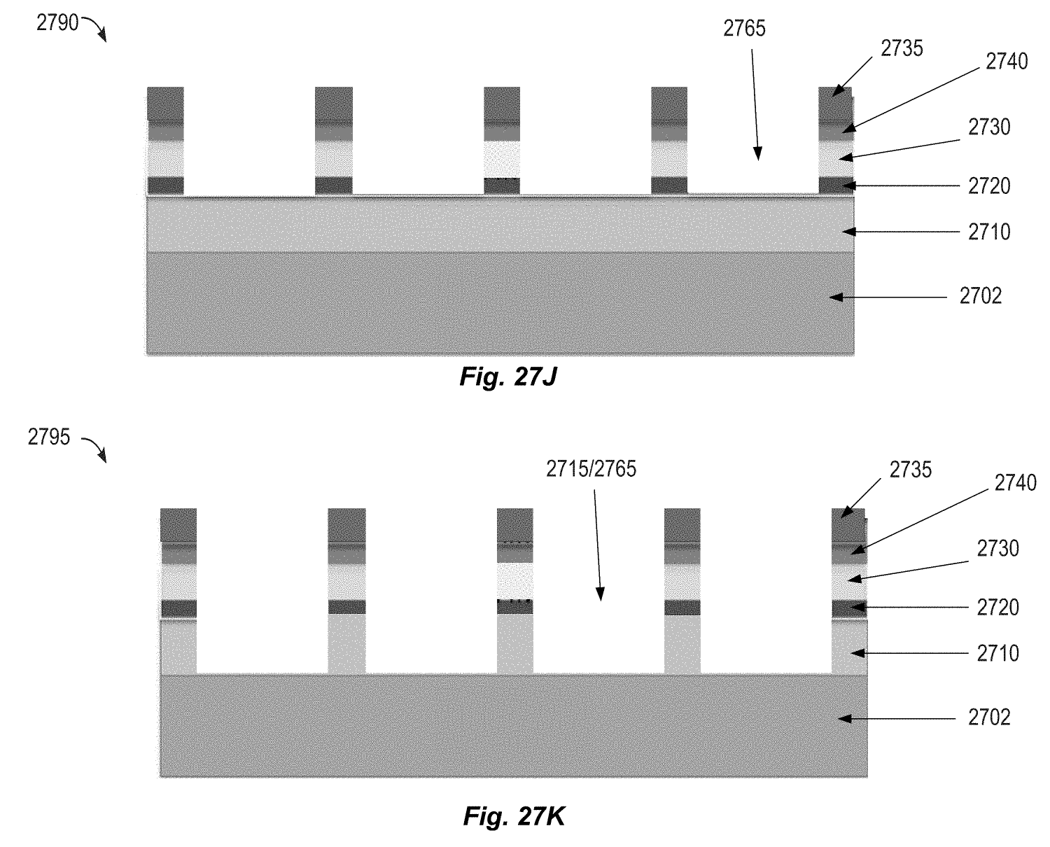

FIG. 26 is a flow diagram illustrating one example process 2600 of fabricating microfluidic chips having electrically-controllable through holes that are substantially similar to the electrode/through hole pairs described with reference to FIGS. 3A, 3B, 20A, 20B, and 23. FIGS. 27A through 27K show example partial cross-sectional side view illustrations of corresponding stages of the fabrication process 2600. FIGS. 27A through 27K are a schematic representation and are not drawn to scale. The features and aspects disclosed herein are intended to be illustrative and may be exaggerated in size to better illustrate the particular aspects of the embodiments described in each representative figure.

While the shapes and dimensions of the respective electrodes and through holes may differ from the non-limiting examples illustrated in FIGS. 26 through 27K, methods of fabricating embodiments of microfluidic chips described herein involve similar features. Therefore, the following describes a method of fabricating a microfluidic chip having electrically-controllable through holes with reference to the microfluidic chip having electrode lines and circular through holes as described in reference to FIG. 26 through 27K, but it will be understood that the same or a substantially similar process may be performed to develop a microfluidic chip having differently-shaped and differently dimensioned electrodes and through holes. Additionally, the steps illustrated in the FIG. 26 flow chart are preferably performed in the illustrated order; however, as will be understood by those skilled in the art, they may also be performed in other sequences and various substitutions and replacements may be made. In the discussion below, some of the possible substitutions and replacements will be discussed in further detail. Further, while omitted in the following description of process 2600, appropriate cleaning steps can be performed periodically and as needed to prepare a given layer for a following processing step and/or to clean the layer based on the previously processed step.

As used herein, the term “wafer” will be used to describe an incomplete microfluidic chip, and the term “microfluidic chip” will be used to describe the completed integrated microfluidic chip. For example, FIGS. 27A through 27I each illustrate one embodiment of a stage of fabricating an integrated microfluidic chip, where wafer 2700 refers to each stage in the process 2600. For example, FIG. 27J and 27K illustrate embodiments of the completed microfluidic chips 2790 and 2795, respectively, fabricated using the process 2600, where each of FIGS. 27A through 27I represents at least one stage of the fabrication process that concludes with the microfluidic chip 2790 of FIG. 27J and the microfluidic chip 2795 of FIG. 27K.

The process 2600 begins at block 2601, where a substrate 2702 is provided as shown in FIG. 27A. The substrate 2702 can be formed of any suitable material and have any suitable dimension to support the filter membrane formed later in the process 2600. Substrate 2702 may be substantially similar to substrate 602 described with reference to FIGS. 5 through 22B or to substrate 2202 described with reference to FIGS. 23 through 25I. In some cases, the substrate 2702 is a silicon wafer. The thickness of the substrate 2702 can be selected based on the needs of the particular application for which the microfluidic chip is intended. The substrate 2702 may be manufactured using microfabrication techniques that are substantially similar to those described with reference to block 501 of FIG. 5.

At block 2602 a filter membrane layer 2710 is deposited on a surface of the substrate 2702. The filter membrane layer 2710 can be substantially similar to filter membrane layer 610 described with reference to FIGS. 5 through 22B or to filter membrane layer 2210 described with reference to FIGS. 23 through 25I. For example, the filter membrane layer 2710 may include any suitable dielectric material that provides suitable transparency, strength, and other physical properties for the intended cell capturing application, as described in more detail with reference to FIGS. 1A and 1B above. Specific requirements of these properties according to at least one embodiment disclosed herein are described above with reference to FIGS. 1A and 1B.

Once a suitable material is selected for the filter membrane layer 2710, deposition of the material may be carried out in a substantially similar manner as described in reference to block 511 of FIG. 5. For example, filter membrane layer 2710 may be formed using deposition techniques such as PVD, PECVD, thermal CVD, E-Beam evaporation, or spin-coating. The filter membrane layer 2710 can be formed to have any suitable thickness for the particular application of the microfluidic chip. The filter membrane layer 2710 includes a first surface 2712 and a second, opposing surface 2714.

In some embodiments, once the filter membrane layer 2710 is deposited, the filter membrane layer 2710 may also be processed to form through holes (not shown). For example, circular through holes may be formed into the filter membrane layer 2710, as described above with reference to sub-process 510 of FIG. 5, and as will be described in more detail below with reference to FIG. 27J. In other embodiments, the through holes are not formed in filter membrane layer 2710 until a later processing step, as will be described in more detail below with reference to FIG. 27K. In either embodiment, the formation and processing of the electrodes follow the same process. Therefore, the following description does not differentiate between either the FIG. 27J or the FIG. 27K embodiment, unless otherwise stated.

The process 2600 then moves to sub-process 2610 where the column connection structure 2720 is formed, as illustrated in FIG. 27B. For example, sub-process 2610 represents the formation of column connection structure that comprises a column contact pad 2721 in electrical communication with column electrodes 2725a-2725n, via column vertical lead lines 2722a-2722n and column connection line 2723. With the formation of the column connection structure 2720, an electrical current or voltage can be selectively and independently applied to each column electrode 2725a-2725n. Therefore, the column vertical lead lines 2722a-2722n can be configured to apply an electrical current to each individual column electrode 2725a-2725n, thereby controlling the voltage bias at each column electrode 2725a-2725n, as described above with reference to block 460 of FIG. 4. The embodiment illustrated in FIG. 27B depicts a four-by-four grid-like pattern comprising five column vertical lead lines with each column vertical lead line having four column electrodes, where one of 16 through holes (not shown) is located between column electrodes of adjacent column vertical lead lines along an x-axis of the device. Other configurations are possible, for example, a 16-by-16 grid or a 2-by-2 grid.

The sub-process 2610 starts at block 2611 with the deposition of a first conductive material layer 2703 on the first surface 2712 of filter membrane layer 2710, as illustrated in FIG. 27A. The deposition of first conductive material layer 2703 is substantially similar to the deposition of conductive layer 503 described in block 503 of FIG. 5, or substantially similar to the deposition of conductive layer 2203 described in block 2403 of FIG. 24. The first conductive material layer 2703 can be formed of any suitable material having the sought after electrical properties. Exemplary conductive materials include gold, platinum, indium tin oxide, titanium nitride, etc. Deposition of the conductive material may be carried out using deposition techniques as described above.

The sub-process 2610 then moves to block 2612 where a photoresist layer 2706 is deposited onto first conductive material layer 2703, as shown in FIG. 27A. The deposition of photoresist layer 2706 may be performed in a substantially similar manner as described above with reference to photoresist layer 606 of FIG. 8, or in a substantially similar manner as described above with reference to photoresist layer 2211 of FIG. 25A. After depositing photoresist layer 2706, a mask (not shown) defining the column connection pattern (not shown) is applied over the photoresist layer 2706. The column connection pattern will be processed in subsequent processing steps to form column connection structure 2720. In the embodiment illustrated in FIG. 27B, the mask defines a column connection pattern in the photoresist layer 2706 that comprises a column contact pad 2721, column vertical lead lines 2722a-2722n, column electrodes 2725a-2725n, and column connection line 2723. In one embodiment, the mask applied to the photoresist layer 2706 is a negative of the column connection pattern such that the intended column connection pattern is covered by the negative mask. Other configurations are possible. The wafer 2700 having the mask applied to the photoresist layer 2706 is then exposed and developed, such that the remaining photoresist layer 2706 defines the column connection pattern. In another non-limiting embodiment, patterning of the column connection structure may be performed using a lift-off technique.

The sub-process 2610 continues to block 2613 with the formation of column connection structure 2720. Formation of the column connection structure 2720 is performed by etching away or removing portions of the first conductive layer 2703 that will not form part of a finished column connection structure 2720, where the portions are defined as the areas that remain uncovered by photoresist layer 2706. Etching may be performed by numerous methods including, as described above, where the etching removes areas of the first conductive layer 2703 unprotected by the remaining photoresist layer 2706. In this step, the filter membrane layer 2710 may act as an etch stop, whereby the etching process is stopped when it reaches the filter membrane layer 2710.

The sub-process 2610 continues to block 2614 with the removal of the photoresist layer 2706 defining the column connection pattern in order to expose the column connection structure 2720. The column connection structure 2720 comprises the remaining material left of the first conductive layer 2703 after etching in block 2612. Once the remaining portions of the photoresist layer 2706 are removed, the column connection structure 2720 formed from the selected conductive material is complete as illustrated in FIG. 27B. The resulting wafer 2700 includes the column connection structure 2720 disposed on the first side 2712 of the filter membrane layer 2710 and comprises column contact pad 2721 in electrical communication with column electrodes 2725a-2725n, via column vertical lead lines 2722a-2722n and column connection line 2723, where the filter membrane layer 2710 is exposed elsewhere.

The process 2600 then moves to block 2620 with the deposition of a first inter-conductive dielectric (IMC1) layer 2730 over the column connection structure 2720 and filter membrane layer 2710, as illustrated in FIG. 27C. The deposition of IMC1 layer 2730 is substantially similar to the deposition of filter membrane layer 2710 described in block 2602. The IMC1 layer 2730 can be formed of any suitable material having the sought after electrical properties. Examples include but are not limited to silicon oxide, silicon oxynitride, silicon nitride, all from organic or inorganic precursors, or from spin-on dielectric materials or precursors. Deposition of the dielectric material may be carried out using deposition techniques as described above.

The process 2600 then moves to block 2630 with the planarization of the IMC1 layer 2730. The planarization is configured to smooth the exposed surface of the IMC1 layer 2730 to correct for uneven features or irregularities resulting from the deposition of the IMC1 layer. In some embodiments, the planarization may be performed by chemical-mechanical planarization (CMP) techniques.

Once the IMC1 layer 2730 is planarized, the process 2600 continues to sub-process 2640 where a row connection structure 2740 is formed that may be functionally similar to column connection structure 2720 described with reference to FIG. 27B. The steps of sub-process 2640 are substantially similar to the steps of sub-process 2610, however, a second conductive material layer 2704 is deposited on the IMC1 layer 2730 instead of the filter membrane layer 2710. Further the features, materials, and properties of the row connection structure 2740 may be substantially similar to those of column connection structure 2720. However, row connection structure 2740 comprises a row contact pad 2741 in electrical communication with row electrodes 2745a-2745n, via row horizontal lead lines 2742a-2742n and row connection line 2743. With the formation of the row connection structure 2740, an electrical current or voltage can be selectively and independently applied to each row electrode 2745a-2745n. Therefore, the row horizontal lead lines 2742a-2742n can be configured to apply an electrical current to each individual row electrode 2745a-2745n, thereby controlling the voltage bias at each row electrode 2745a-2745n, as described above with reference to block 460 of FIG. 4. The embodiment illustrated in FIG. 27D depicts a four-by-four grid-like pattern comprising five row horizontal lead lines with each row horizontal lead line having four row electrodes, where one of 16 through holes (not shown) is located between row electrodes of adjacent row horizontal lead lines along an y-axis of the device. Other configurations are possible, for example, a 16-by-16 grid or a 2-by-2 grid.

The sub-process 2640 starts at block 2641 with the deposition of the second conductive material layer 2704 on the surface of IMC1 layer 2730. The deposition of the second conductive material layer 2704 is substantially similar to the deposition of first conductive material layer 2703 described in block 2611. The second conductive material layer 2704 can be formed of any suitable material having the sought after electrical properties. Exemplary conductive materials include gold, platinum, indium tin oxide, titanium nitride, etc. Deposition of the conductive material may be carried out using deposition techniques as described above.

The sub-process 2640 then moves to block 2642 where a photoresist layer (not shown) is deposited onto the second conductive material layer. The deposition of the photoresist layer may be performed in a substantially similar manner as described above with reference to photoresist layer 2706. After depositing the photoresist layer, a mask (not shown) defining the row connection pattern (not shown) is applied over the photoresist layer. The row connection pattern will be processed in subsequent processing steps to form row connection structure 2740. In the embodiment illustrated in FIG. 27D, the mask defines the row connection pattern in the photoresist layer that comprises a row contact pad 2741, row horizontal lead lines 2742a-2742n, row electrodes 2745a-2745n, and row connection line 2743. In one embodiment, the mask applied to the photoresist layer is a negative of the row connection pattern such that the intended row connection pattern is covered by the negative mask. Other configurations are possible. The wafer 2700 having the mask applied to the photoresist layer is then exposed and developed, such that the remaining photoresist layer defines the row connection pattern. In another non-limiting embodiment, patterning of the row connection structure may be performed using a lift-off technique.

The sub-process 2640 continues to block 2643 with the formation of row connection structure 2740. Formation of the row connection structure 2740 is performed by etching away or removing portions of the second conductive material layer that will not form part of a finished row connection structure 2740, where the portions are defined as the areas that remain uncovered by photoresist layer. Etching may be performed by numerous methods including, as described above, where the etching removes areas of the second conductive material layer 2704 unprotected by the remaining photoresist layer. In this step, the IMC1 layer 2730 may act as an etch stop, whereby the etching process is stopped when it reaches the IMC1 layer 2730.

The sub-process 2640 continues to block 2644 with the removal of the photoresist layer defining the row connection pattern in order to expose the row connection structure 2740. The row connection structure 2740 comprises the remaining material left of the second conductive material layer 2704 after etching in block 2644. Once the remaining portions of the photoresist layer are removed, the row connection structure 2740 formed from the selected conductive material is complete as illustrated in FIG. 27D and FIG. 27E. FIG. 27F illustrates a partial cross-sectional view of the wafer 2700, taken along the line A-A illustrated in FIG. 27E. As depicted in FIGS. 27D through 27F, the resulting wafer 2700 includes the row connection structure 2740 being disposed on the IMC1 layer 2730 and comprises row contact pad 2741 in electrical communication with row electrodes 2745a-2745n, via row horizontal lead lines 2742a-2742n and row connection line 2743, with the IMC1 layer 2730 is exposed elsewhere.

FIG. 27D illustrates a top down view of wafer 2700 illustrating only the row connection structure 2740 on the IMC1 layer 2730. FIG. 27E illustrates a similar top down view of wafer 2700, however, the schematic illustration of FIG. 27E depicts the alignment of the column connection structure 2720 relative to the row connection structure 2740. In one embodiment, the IMC1 layer 2730 is transparent, thereby permitting the column row structure 2720 to be viewable through the IMC1 layer 2730. As illustrated in FIG. 27E, the row connection structure 2740 and column connection structure 2720 are precisely aligned to form a grid-like pattern on wafer 2700. In illustrated embodiment, the column connection line 2723 is located along a first edge of the wafer 2700 and the row connection line 2743 is located along a second edge of the wafer 2700, where the first and second edges form approximately a right angle. Other configurations are possible. In this way, the column connection structure 2720 is configured to control the electrical current or voltage applied to precisely-identified column electrodes 2725a-2725n along each of the column vertical lead lines 2722a-2722n. The row connection structure 2740 is configured to control the electrical current or voltage applied to precisely-identified row electrodes 2745a-2745n along each of the row horizontal lead lines 2742a-2742n. By selectively applying a current or voltage along a given column vertical lead line and a given row horizontal lead line, a pair of electrodes comprising one column electrode 2725 and one row electrode 2745 can be identified and controlled. In some embodiments, a through hole (not shown) is positioned in each opening of the grid-like pattern, and by selectively controlling a given column electrode and a given row electrode, the voltage applied to a through hole associated with the given column electrode and the given row electrode is also controlled. A voltage bias may be applied to any contents captured, retained, or disposed within the through hole, as described in more detail above with reference to operation 460 of FIG. 4.

The process 2600 then moves to block 2650 with the deposition of a second inter-conductive dielectric (IMC2) layer 2735 over the row connection structure 2740 and IMC1 layer 2730, as illustrated in FIG. 27G. The deposition of IMC2 layer 2735 is substantially similar to the deposition of IMC1 layer 2730. The IMC2 layer 2735 can be formed of any suitable material having the sought after electrical properties. Examples include but are not limited to silicon oxide, silicon oxynitride, silicon nitride, all from organic or inorganic precursors, or from spin-on dielectric materials or precursors. Deposition of the dielectric material may be carried out using deposition techniques as described above. The process 2600 then moves to block 2655 with the planarization of the IMC2 layer 2735. The planarization is configured to smooth the exposed surface of the IMC2 layer 2735 in a substantially similar manner as performed on IMC1 layer 2730.

The process 2600 continues to block 2660 with the deposition of a hard mask layer 2750 on to IMC2 layer 2735, as illustrated in FIG. 27G. Deposition of the hard mask layer 2450 may be carried out using deposition techniques such as PVD, PECVD, thermal CVD, E-Beam evaporation, or spin-coating a thin layer of the selected hard mask material onto substrate IMC2 layer 2735. In some embodiments, the material of the hard mask layer 2750 is resistant to hydrofluoric acid (HF). Example hard mask materials include amorphous silicon (a-Si). Other materials are possible.

The process 2600 continues to block 2665 where a photoresist layer 2755 is deposited onto the hard mask layer 2750, as shown in FIG. 27G. The deposition of the photoresist layer 2755 may be may be performed in a substantially similar manner as described above with reference to photoresist layer 2706. However, the photoresist layer 2755 is configured to define via holes 2765 in the hard mask layer 2750 by defining via hole patterns 2760 in the photoresist layer 2755. After depositing the photoresist layer 2755, a mask (not shown) defining the via hole patterns 2760 is applied over the photoresist layer 2755. In one embodiment, the mask applied to the photoresist layer 2755 is a negative of the via holes 2765 such that the intended via hole patterns 2760 are covered by the negative mask. Other configurations are possible.

The process 2600 then moves to block 2670 where the via hole patterns 2760 are formed in the photoresist layer 2755. The process of forming the via hole patterns 2760 in the photoresist layer 2755 is substantially similar to the formation of the column connection patterns in the photoresist layer 2706 (for example, exposing the masked photoresist layer and performing a development step to remove the mask).

The process 2600 then moves to block 2675 with the formation of the via holes patterns 2760 in the hard mask layer 2750, as illustrated in FIGS. 27G and 27H. Formation of the via holes patterns 2760 in the hard mask 2750 is performed by etching away or removing portions of the hard mask layer 2750 defined as areas that remain uncovered by the photoresist layer 2755. Specifically, the portions of the hard mask 2750 that are removed in block 2675 are the portions of the hard mask layer 2750 that were exposed when portions of photoresist layer 2755 were removed during the developing stage of block 2770. In block 2675, the IMC2 layer 2735 may act as an etch stop, whereby the etching process is stopped when it reaches the IMC2 layer 2735.

The resulting wafer 2700 is illustrated in FIGS. 27G and 27H where the remaining photoresist layer 2755 defines via hole patterns 2760 in the hard mask layer 2750. FIG. 27G is a partial schematic cross-sectional side view illustrating the wafer 2700 as fabricated up to block 2675, including the via hole patterns 2760 into the photoresist layer 2755 and hard mask 2750. FIG. 27H illustrates a partial top down view illustrating the wafer 2700 according to FIG. 27G, where the via hole patterns 2760 are positioned between row electrodes 2745a-2745n and column electrodes 2722a-2722n, and IMC2 layer 2735 is viewable in the via hole patterns 2760.

The process 2600 continues to block 2680 with the removal of the photoresist layer 2755 defining the via hole patterns 2760 in order to expose the hard mask layer 2750. The patterned hard mask layer 2750 comprises the remaining material left of the hard mask layer 2750 after etching in block 2675. Once the remaining portions of the photoresist layer 2755 are removed, the via hole patterns 2760 can be formed in the hard mask layer 2750.

The process 2600 then moves to block 2685 with the formation of the via holes 2765 in the IMC1 layer 2730 and IMC2 layer 2735, as illustrated in FIGS. 27I through 27K. Formation of the via holes 2765 is performed by etching away or removing portions of the IMC1 layer 2730 and IMC2 layer 2735 defined as areas that remain uncovered by the hard mask layer 2750 having via hole patterns 2760. In this regard, portions of the IMC1 layer 2730 and IMC2 layer 2735 that will not form part of wafer 2700 are etched away or removed. Specifically, the portions of the IMC1 layer 2730 and IMC2 layer 2735 that are removed in block 2685 are the portions of the IMC1 layer 2730 and IMC2 layer 2735 that were exposed when portions of hard mask layer 2750 were removed during the forming of via hole patterns 2760 in the hard mask layer 2750 at block 2675. In block 2685 of some embodiments, the filter membrane layer 2710 may act as an etch stop, whereby the etching process is stopped when it reaches the filter membrane layer 2710, as illustrated in FIG. 27J. In this instance, through holes (not shown) may have been previously processed in filter membrane layer 2710 and are thereby exposed during the formation of via holes 2765 in block 2785. In other embodiments, the substrate 2702 may act as an etch stop, whereby the etching process is stopped when it reaches the substrate 2702, as illustrated in FIG. 27K. In this instance, the through holes 2715 may be formed in block 2685 during formation of the via holes 2765 in IMC1 layer 2730 and IMC2 layer 2735.

The process 2600 continues to block 2690 with exposure of the column and row electrodes 2725a-2725n and 2745a-2745n, respectively, within the via holes 2765, as illustrated in FIG. 27I. In one embodiment, exposure of the electrodes is performed by enlarging the via holes 2765, thereby exposing a portion of each electrode in via hole 2765 to the surrounding environment. One implementation of exposing a portion of the electrodes is to dip the wafer 2700 into dilute HF acid. The dilute HF acid will remove a portion of the IMC1 layer 2730 and the IMC2 layer 2735 but will leave the electrodes unaffected. By using the hard mask layer 2750, the surface of the IMC2 layer 2735 that is protected by the hard mask layer 2750 is unaltered by the HF acid. This permits only the area of the IMC1 layer 2730 and IMC2 layer 2735 that is exposed within the via holes 2765 to be in contact with the HF acid. In this way, the diameter of each via hole 2765 can be enlarged. The size and shape of the via holes 2765 fabricated in the IMC1 layer 2730 and IMC2 layer 2735 can be selected based on the needs of the particular application for which the microfluidic chip is intended. It is noted that the embodiment illustrated in FIG. 27I is substantially similar to FIG. 27J, where the filter membrane layer 2710 functions as an etch stop in block 2685, thus the filter membrane layer 2710 is viewable through the via holes 2765. In the illustrated embodiment, through holes are not shown in the filter membrane layer 2710. In some embodiments, the through holes may be formed in the filter membrane layer 2710 prior to sub-process 2610, while in other embodiments the through holes may be processed subsequent to exposing the column and row electrodes 2725a-2725n and 2745a-2745n.

Once the column and row electrodes are exposed, the process 2600 continues to block 2695, with the removal of the hard mask layer 2750 in order to expose the IMC2 layer 2735 and proceed with finalizing the wafer 2700. The wafer 2700 comprises the remaining material left of the multiple layer stack after dipping the wafer 2700 in the HF acid. In some embodiments, removal of the remaining portions of the hard mask layer 2750 is performed by chemical processes (i.e., dry plasma or wet chemistry) selective to underlying layers. Once the remaining portions of the hard mask layer 2750 are removed, the column electrodes 2725a-2725n and row electrodes 2745a-2745n formed from the respective conductive material layers are complete as illustrated in FIGS. 27I through 27K.

Through precise processing and alignment during the above-described processing steps, the through holes can be precisely aligned with the electrodes 2725a-2725n and 2745a-2745n. In the example implementation illustrated in FIG. 27K, precise alignment results in a single through hole 2715 associated with at least one column electrode and row electrode. This precise alignment permits individual electrode/through hole pairs to be precisely identified based on their distinct, precisely-defined location within the filter membrane layer 2710 (which corresponds to a filter membrane as described herein). By precisely aligning and positioning each electrode relative to its corresponding through hole, the electrode/through hole pairs enable precise and independent control of a voltage bias applied to each through hole and any contents therein, as described above with reference to FIGS. 3A through 4. For example, a first electrical signal may be communicated to the column control pad 2721, which is configured to send a voltage across column connecting line 2723 to a selected one of the multiple column vertical lead lines 2722a-2722n. Similarly, a second electrical signal may be communicated to the row control pad 2741, which is configured to send a voltage across row connecting line 2743 to a selected one of the multiple row horizontal lead lines 2745a-2745n. In this way, a voltage bias is applied to the electrodes positioned on the grid where the column vertical lead line and row horizontal lead line cross. This enables precise and independent control of a voltage bias applied to each through hole and any contents therein.

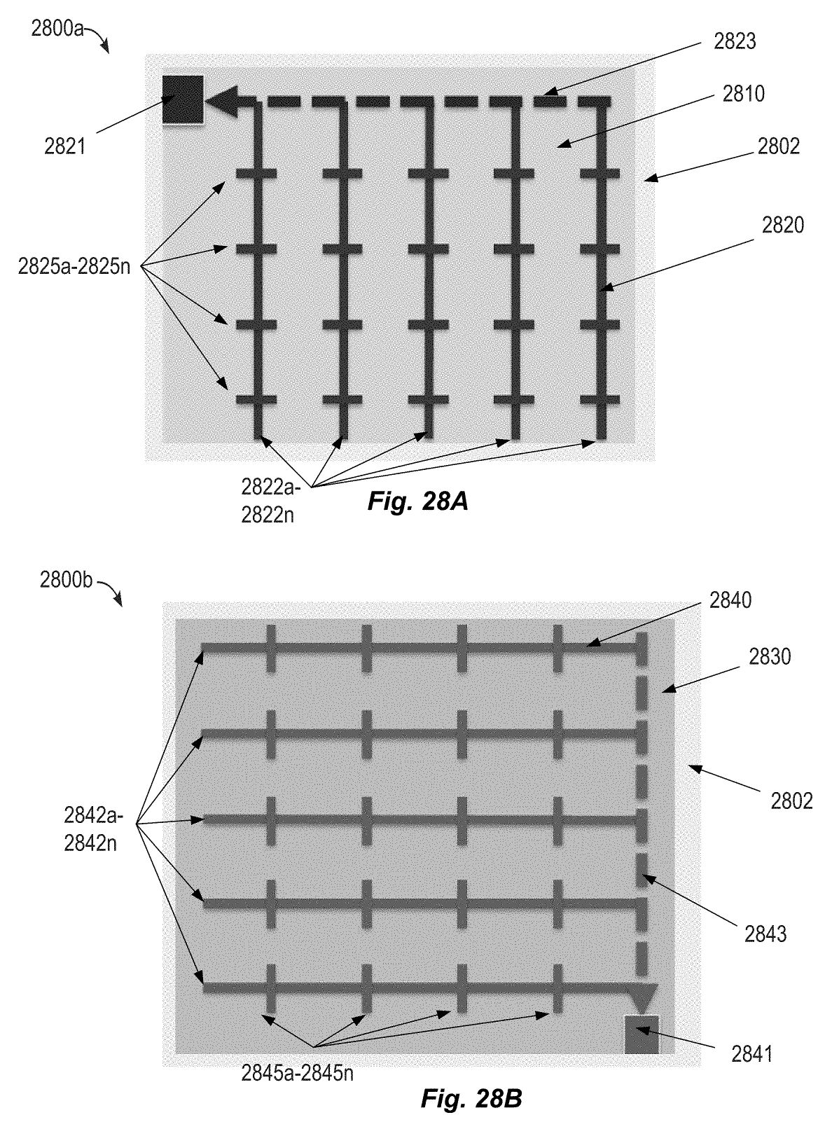

FIGS. 28A and 28B illustrate another embodiment of fabricating a microfluidic chip that is substantially similar to microfluidic chip 2790 illustrated in FIG. 27J and/or microfluidic chip 2795 illustrated in FIG. 27K. The microfluidic chips illustrated in FIGS. 28A and 28B comprises two substrates 2800a and 2800b, where each substrate includes one of a row connection structure (for example, a row connection structure substantially similar to row connection structure 2740) and a column connection structure (for example, a column connection structure substantially similar to column connection structure 2720).

As illustrated in FIG. 28A, a first substrate 2802a is provided having a column connection structure 2820 embossed or embedded in the first substrate 2802a. As illustrated in FIG. 28b, a second substrate 2802b is provided having a row connection structure 2840 embossed or embedded in the second substrate 2802b. The two substrates are then aligned and bonded such that the column connection structure 2820 and row connection structure 2840 are positioned and are functionally similar to that described above with reference to FIGS. 27I and 27K. In some embodiments, the first and second substrates 2802a and 2802b are a suitable material for the embossing or embedding of a conductive metal layer and a material selected that may be bonded upon a similar material, for example thin glass substrates of several microns thick may be used. The row and column connection structures 2820 and 2840 may be of a suitable conductive material as described in the above embodiments.

Once the first and second substrates 2802a and 2802b are bonded together, via hole patterns are etched through the first and/or second substrate 2802a and 2802b, as described above, to expose the row and column electrodes. Without being bound to any particular theory, it is believed that the techniques of localized laser damaging of a glass substrate prior to a wet etch of the substrates can enhance localized ER more than 30 times in the exposed areas. In one embodiment, the via holes may be etched completely through the first and second substrate 2802a and 2802b, thereby creating a through-silicon via hole such that a vertical electrical connection passes completely through the first and second substrates 2802a and 2802b. In another embodiment, the etching may be performed partially through one or both substrates 2802a and 2802b, or completely through one substrate and partially through the other substrate. Electrical connection of the electrodes to a control system is performed through the metal contact pads 2821 and 2841, which are substantially similar to column contact pad 2721 and/or row contact pad 2741 of the embodiment illustrated in FIGS. 27A through 27K. Without being bound to any particular theory, it is believed that the fabrication in this way on thin glass substrates enhances and facilitates wafer level manufacturing and packing.

It is noted that FIGS. 28A and 28B are schematic representations and are not drawn to scale. The features and aspects disclosed herein are intended to be illustrative and may be exaggerated in size to better illustrate the particular aspects of the embodiments described in each representative figure.

Those having skill in the art will further appreciate that the various illustrative logical blocks, modules, circuits, and process steps described in connection with the implementations disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present embodiments. A person of ordinary skill in the art will appreciate that a portion, or a part, may comprise something less than, or equal to, a whole. For example, a portion of a collection of pixels may refer to a sub-collection of those pixels.

The steps of a method or process described in connection with the implementations disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of non-transitory storage medium known in the art. An exemplary computer-readable storage medium is coupled to the processor such the processor can read information from, and write information to, the computer-readable storage medium. In the alternative, the storage medium may be integral to the processor. The processor and the storage medium may reside in an ASIC. The ASIC may reside in a user terminal, camera, or other device. In the alternative, the processor and the storage medium may reside as discrete components in a user terminal, camera, or other device.

Headings are included herein for reference and to aid in locating various sections. These headings are not intended to limit the scope of the concepts described with respect thereto. Such concepts may have applicability throughout the entire specification.

The previous description of the disclosed implementations is provided to enable any person skilled in the art to make or use the embodiments described herein. Various modifications to these implementations will be readily apparent to those skilled in the art, and the generic principles defined herein may be applied to other implementations without departing from the spirit or scope of the embodiments. Accordingly, the disclosed embodiments are not intended to be limited to the implementations shown herein but instead are to be accorded the widest scope consistent with the principles and novel features disclosed herein.

1. A device, comprising:
a filter structure comprising a plurality of through holes extending from a first side of the filter structure to a second side of the filter structure and arranged in a repeating pattern, each of the through holes having a first opening on the first side of the filter structure, a second opening on the second side of the filter structure, and a passageway through the filter structure between the first opening and the second opening, the first and second openings sized to capture an object in the through hole;
a substrate including a plurality of vanes that supports at least a portion of the filter structure, the filter structure disposed relative to the plurality of vanes such that the second side of the filter structure is adjacent to the plurality of vanes;
a plurality of electrodes comprising a set of electrodes associated with each through hole, each set of electrodes including at least a pair of electrodes associated with each through hole, each set of electrodes aligned with its associated through hole to apply electrical forces to an object captured in the through hole, each set of electrodes and associated through hole having a distinct, precisely-defined location in the filter structure; and
electrical connections to each of the plurality of electrodes, the electrical connections and plurality of electrodes collectively configured to communicate electrical signals to the plurality of electrodes from a controller connected to the device for independently controlling the application of electrical forces through each set of electrodes to an object in the associated through hole.

2. The device of claim 1, wherein for each pair of electrodes and associated through hole, a first electrode of the pair of electrodes is positioned on the through hole on the first side of the filter structure, and a second electrode of the pair of electrodes is positioned on the through hole on the second side of the filter structure.

3. The device of claim 1, wherein for each pair of electrodes and associated through hole, both a first electrode and a second electrode of the pair of electrodes are positioned on the first side of the filter structure.

4. The device of claim 1, wherein for each pair of electrodes and associated through hole, both a first electrode and a second electrode of the pair of electrodes are positioned on the second side of the filter structure.

5. The device of claim 1, wherein each of the electrodes of the pair of electrodes are ring-shaped.

6. The device of claim 5, wherein the through holes are oval-shaped.

7. The device of claim 1, wherein each of the electrodes of the pair of electrodes are diamond-shaped.

8. The device of claim 7, wherein the through holes are circular-shaped.

9. The device of claim 1, wherein the set of electrodes includes three electrodes.

10. The device of claim 1, wherein the set of electrodes includes four electrodes.

11. The device of claim 1, wherein the set of electrodes is configured to apply an electrical force to an object in the associated through hole to fragment an object in the through hole.

12. The device of claim 1, wherein the set of electrodes is configured to apply an electrical force to an object in the associated through hole to change the shape of an object in the through hole.

13. The device of claim 1, wherein the set of electrodes is configured to apply an electrical force to an object in the associated through hole to repel an object from the through hole.

14. The device of claim 1, wherein the set of electrodes is configured to apply an electrical force to attract an object into the associated through hole.

15. The device of claim 1, wherein the electrical connections comprise a column connection structure including a column contact pad electrically connected to a column connection line, and a plurality of column lead lines electrically connected to the column connection line, the plurality of column lines each connected to at least one electrode aligned with each through hole.

16. The device of claim 1, wherein the electrical connections comprise a row connection structure including a row contact pad electrically connected to a row connection line, a plurality of row lead lines electrically connected to the row connection line, the plurality of row lines connected to at least one electrode aligned with each through hole.

17. The device of claim 1, wherein the filter structure is formed on the substrate.

18. The device of claim 1, wherein the filter structure has a thickness in the range of about 1 μm to about 20 μm.

19. The device of claim 1, wherein the second opening is smaller than the first opening, and wherein the first and second openings have a first dimension of between about 4 μm and about 10 μm and a second dimension of between about 4 μm and about 10 μm.

20.-34. (canceled)

35. method of capturing an object in a through hole, the method comprising:
capturing an object in a through hole of a device including a filter structure having plurality of through holes extending from a first side of the filter structure to a second side of the filter structure and arranged in a repeating pattern, each of the through holes having a first opening on the first side of the filter structure, a second opening on the second side of the filter structure, and a passageway through the filter structure between the first opening and the second opening, the first and second openings sized to capture an object in the through hole, the device further including a substrate having a plurality of vanes that supports at least a portion of the filter structure, the filter structure disposed relative to the plurality of vanes such that the second side of the filter structure is adjacent to the plurality of vanes; and
applying an electrical force to the captured object using a plurality of electrodes comprising a set of electrodes associated with each through hole, each set of electrodes including at least a pair of electrodes associated with each through hole, each set of electrodes aligned with its associated through hole to apply electrical forces to the object captured in the through hole, each set of electrodes and associated through hole having a distinct, precisely-defined location in the filter structure.

36. The method of claim 35, wherein applying the electrical force comprises applying an electrical force to an object in the associated through hole to fragment an object in the through hole.

37. The method of claim 35, wherein applying the electrical force comprises applying electrical force to an object in the associated through hole to change the shape of an object in the through hole.

38. The method of claim 35, wherein applying the electrical force comprises applying an electrical force to attract an object into the associated through hole.

39. The method of claim 35, wherein applying the electrical force comprises applying electrical force to an object in the associated through hole to repel an object from the through hole.
